(19) United States
(12) Patent Application Publication
(10) Pub. No.: US 2026/0151434 A1
(43) Pub. Date: Jun. 4, 2026

(54) COMPOSITIONS FOR THE TREATMENT OF DISEASE

(71) Applicant: SightStream Biotherapeutics, Inc., Dallas, TX (US)

(72) Inventors: Mark Christopher Jasek, Fort Worth, TX (US); Norris Andrew Whitlock, Mount Pleasant, SC (US); Jiagang Zhao, San Diego, CA (US); Renato Skerlj, Newton, MA (US)

(21) Appl. No.: 19/100,395

(22) PCT Filed: Aug. 2, 2023
(86) PCT No.: PCT/US2023/071502

§ 371 Date: Jan. 31, 2025

Related U.S. Application Data
(60) Provisional application No. 63/394,875, filed on Aug. 3, 2022.

(51) Int. Cl. A61K 35/30; A61K 9/00; A61K 38/34; A61P 27/02; C07K 14/685; C12N 5/079; C12N 5/10; C12N 15/867

(52) U.S. Cl. CPC A61K 35/30; A61K 9/0014; A61K 9/0048; A61K 38/34; A61P 27/02; C07K 14/685; C12N 5/0621; C12N 5/10; C12N 15/867; C12N 2510/00; C12N 2740/15043; C12N 2830/34

(57) ABSTRACT

The disclosure relates to compositions and methods comprising MSH analogs and cell therapy.

### CROSS-REFERENCE TO RELATED APPLICATION(S)

This application claims priority to, and the benefit of, U.S. Provisional Application No. 63/394,875, filed Aug. 3, 2022. The disclosures of said provisional application are hereby incorporated by reference in their entirety.

### STATEMENT REGARDING SEQUENCE LISTING

The Sequence Listing associated with this application is provided in .xml format in lieu of a paper copy and is hereby incorporated by reference into the specification. The name of the .xml file containing the Sequence Listing is KERA-002-WO1_Seq_Listing.xml. The xml file is 275 kb, was created on Jul. 7, 2022, and is being submitted electronically via Patent Center.

### FIELD OF THE DISCLOSURE

The disclosure relates to compositions and methods comprising MSH analogs and cell therapy.

### BACKGROUND OF THE DISCLOSURE

Human endothelial cells were recently found to express certain melanocortin receptors (MCRs), including the MC1-receptor (MC1-R). These endothelial MC1-receptors thus have great potential for being targeted in the treatment of various ophthalmic indications, However, many melanocortins (and analogs thereof) have limited selectivity between various MCRs and tissue types, with few MCR-specific peptides having been identified as effective therapeutics or approved for pharmaceutical applications. A strong need therefore exists for novel MCR-targeting compounds (e.g., novel α-MSH peptide analogs) which are highly specific to particular MCR subtypes.

Cell therapies continue to provide patients with promising therapeutic benefits in several disease. However, there remains a need in the art to treat diseases such as inflammatory, autoimmune and immune system disorders. The present disclosure addresses this need by using novel α-MSH analogs and cell therapy to treat a variety of diseases such as inflammatory, autoimmune and immune system disorders diseases, cancers, ocular diseases, nervous system diseases, neurodegenerative diseases, and infections, amongst many others.

### SUMMARY OF THE DISCLOSURE

Disclosed herein are cell therapy methods useful for treating diseases, which methods generally comprise the use, administration and/or co-administration of one or more α-MSH peptide analogs and a cell therapy (e.g., a cell therapy comprising the administration or transplantation of a population of ocular stem cells). In certain embodiments, the methods disclosed herein comprise a step of pre-treating a subject (e.g., pre-treating for 1 week, 2 weeks, 3 weeks, 4 weeks or more) by topically or ophthalmically administering one or more of α-MSH peptide analogs to the subject (e.g., one or more α-MSH peptide analogs selected from the group consisting of SEQ ID NOs: 1-79). In certain embodiments, the methods comprise a further step of administering or transplanting a population of cells (e.g., isolated mammalian primitive retinal stem cells) in the subject (e.g., intraocular administration or transplantation of such population of cells). In certain aspects, the one or more of the α-MSH peptide analogs are administered topically or ophthalmically to the subject for about 7-14 days, 10-21 day, or 14-28 days prior to transplanting the population of cells into the subject.

In certain embodiments, the population of cells (e.g., ocular stem cells) are contacted in vitro or ex vivo with one or more α-MSH peptide analogs prior to being administered or transplanted in the subject, and wherein α-MSH peptide analogs are the selected from SEQ ID NOs: 1-79. Accordingly, also disclosed herein are cell therapy methods comprising the steps of: a. obtaining a population of cells (e.g., isolated mammalian primitive retinal stem cells) from an organism or a cell source; b. contacting the population of cells in vitro or ex vivo with one or more α-MSH peptide analogs selected from SEQ ID NOs: 1-79; and c. transplanting the population of cells into a subject in need thereof.

In some embodiments, the cell therapy methods include expanding the population of cells prior to contacting the population of cells with the one or more α-MSH peptide analogs of SEQ ID NOs: 1-79. In some embodiments, the cell therapy methods include expanding the population of cells after contacting the population of cells with the one or more α-MSH peptide analogs of SEQ ID NOs: 1-79.

In some embodiments, the population of cells (e.g., isolated mammalian primitive retinal stem cells) is treated with one or more α-MSH peptide analogs for less than 24 hours. In some embodiments, the population of cells comprises one or more cell types selected from group consisting of a stem cell, a progenitor cell, and a somatic cell. In some embodiments, the population of cells is autologous, allogeneic, or xenogeneic. In one embodiment, the population of cells comprises a stem cell, wherein the stem cell is an embryonic stem cell, an adult stem cell or an induced pluripotent stem cell. In one embodiment, the population of cells comprises a progenitor cell, wherein the progenitor cell is a neural progenitor cell, a liver progenitor cell, corneal epithelial progenitor cells, a photoreceptor precursor cell, a retinal ganglion precursor cell, a monoblast cell, myeloblast cell, or a hematopoietic progenitor cell. In one embodiment, the population of cells comprises a somatic cell, wherein the somatic cell is an immune cell, a fibroblast, a chondrocyte, a keratinocyte, a hepatocyte, or a pancreatic cell.

In some embodiments of any of the inventions disclosed herein, the population of cells comprises ocular stem cells. In some embodiments, the population of cells comprises cell-fate further restricted precursors of ocular stem cells. In one embodiment, the ocular cell-fate further restricted precursors of ocular stem cells comprise a photoreceptor precursor cell. In one embodiment, the ocular cell-fate further restricted precursors of ocular stem cells comprise a retinal ganglion precursor cell. In one embodiment, the ocular cell-fate further restricted precursors of ocular stem cells comprise a retinal pigmented epithelial (RPE) cell. In one embodiment, the ocular cell-fate further restricted precursors of ocular stem cells comprise a corneal endothelial cell.

In certain embodiments, contacting the ocular stem cells disclosed herein with one or more α-MSH peptide analogs increases the expression of one or more melanocortin receptors (MCR) by the ocular stem cell (e.g., ABC-101). For example, in certain embodiments, such expression of MCRs is increased two-, three-, four-, five-, six-, ten-, twelve, twenty-, twenty-five, thirty-, forty-, fifty, sixty-fold or more following contact with one or more α-MSH peptide analogs.

In certain embodiments, pre-treating a subject with one or more α-MSH peptide analogs prior to the administration or transplant of ocular stem cells increases the expression of MCRs in one or more of the subject's endogenous cells that are capable of expressing MCRs. For example, in certain embodiments, such expression of MCRs in the subject's endogenous cells is increased two-, three-, four-, five-, six-, ten-, twelve, twenty-, twenty-five, thirty-, forty-, fifty, sixty-fold or more following contact with one or more α-MSH peptide analogs.

In some embodiments, the population of cells is engineered to express at least one payload. The at least one payload of interest may be one or more of a protein of interest, a fusion polypeptide, an antibody, an antigen, a chimeric antigen receptor (CAR), a T cell receptor (TCR), a safety switch, and/or a regulatory switch. In certain embodiments, the engineered cells express an α-MSH peptide or fragment thereof.

In some embodiments, the population of cells is genetically modified using a gene editing system selected from the group consisting of a CRISPR, a TALEN, a Zn-Finger, and a vector delivery system. In one embodiment, the gene editing system is delivered to a cell via a vector delivery system (e.g., a RNA, DNA, or viral vector delivery system).

In some embodiments, the population of cells are activated prior to transplanting the population of cells (e.g., transplanting the population of isolated mammalian primitive retinal stem cells intraocularly). In some embodiments, the population of cells expresses MCRs.

In some embodiments, the α-MSH peptide analog is selected from the group consisting of SEQ ID NO: 1. SEQ ID NO: 2, SEQ ID NO: 3, and SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11. SEQ ID NO: 12. SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72. SEQ ID NO: 73, SEQ ID NO: 74, and SEQ ID NO: 75.

In some embodiments, the step of contacting the population of cells in vitro or ex vivo with one or more α-MSH peptide analogs comprises engineering the population of cells to create an engineered cell capable of endogenously expressing a transgene encoding the one or more α-MSH peptide analogs.

Also disclosed herein is a cell therapy product produced by the methods described herein.

Further disclosed herein are methods of administering a composition to a subject, wherein the composition comprises a population of cells (e.g., isolated mammalian primitive retinal stem cells) and one or more α-MSH peptide analogs of SEQ ID NO: 1-79. In certain embodiments, the population of cells and one or more α-MSH peptide analogs are co-administered or are administered concurrently. In other embodiments, the one or more α-MSH peptide analogs are administered as a pre-treatment in advance of the administration of the population of cells (e.g., pre-treating a subject by administering one or more α-MSH peptide analogs about 1 day, 3 days, 5 days, 7 days, 10 days, 14 days, 21 days, 28 days, 30 days, 42 days, 60 days or more prior to administering or transplanting the ocular stem cells). The α-MSH peptide analogs disclosed herein advantageously distribute into the subject's tissues following topical or ophthalmic administration, thereby avoiding the need for invasive or painful procedures.

In some embodiments, the population of cells comprises ocular stem cells. In some embodiments, the administration of the population of cells occurs via IVT injection or intraretinal deposit. In some embodiments, the composition is administered to the subject to treat a disease of the eye (e.g., Leber Hereditary Optic Neuropathy (LHON), optic neuropathies, age related macular degeneration, glaucoma, cone-rod dystrophies, corneal endothelial dystrophies, inherited retinal diseases (IRDs), and retinitis pigmentosa (RP)).

Also disclosed herein are methods of preparing a therapeutic composition comprising engineered ocular cells (e.g., engineered ocular stem cells) comprising: a. providing an isolated ocular cell; b. contacting the ocular cell with a transgene expression vector in vitro, thereby introducing the expression vector into the ocular cells to form engineered ocular cells, wherein the transgene encodes an α-MSH polypeptide or fragment thereof, wherein the transgene expression vector comprises cis-regulatory and promoter sequences that control the expression of the transgene; and c. formulating the engineered ocular cells into a suspension for intraocular administration to a subject in need thereof.

In some embodiments, the transgene expression vector is AAV or lentiviral vector in vitro. In some embodiments, the α-MSH polypeptide comprises SEQ ID NO: 76 or a fragment thereof (e.g., a biologically active fragment of an α-MSH polypeptide). In some embodiments, the ocular cell comprises ocular stem cell. In some embodiments, the ocular cell comprises a cell-fate restricted progeny of ocular stem cell. In one embodiment, the ocular cell-fate further restricted precursor cell comprises a photoreceptor precursor cell. In one embodiment, the ocular cell-fate further restricted precursor cell comprises a retinal ganglion precursor cell. In one embodiment, the ocular cell-fate further restricted precursor cell comprises a retinal pigmented epithelial (RPE) cell. In one embodiment, the ocular cell-fate further restricted precursor cell comprises a corneal endothelial cell.

Further disclosed herein are therapeutic compositions for treating an ocular disease or disorder in a human subject in need thereof, comprising: ocular cells that are introduced with a transgene expression vector in vitro, wherein the transgene expression vector comprises cis-regulatory and promoter sequences that control the expression of a transgene encoding an α-MSH polypeptide; and wherein the ocular cells comprising the transgene are formulated into a suspension of cells for intraocular administration to the human subject.

In some embodiments, the transgene expression vector is an AAV or lentiviral vector. In some embodiments, the ocular cells comprising the transgene are then cryopreserved for long-term storage. In one embodiment, the cryopreserved ocular cells are thawed and formulated to a suspension of cells for the intraocular administration to the human subject. In some embodiments, the ocular disease or disorder comprises neovascular AMD.

In some embodiments, the ocular cell comprises ocular stem cells. In some embodiments, the ocular cell comprises an ocular cell-fate further restricted precursor cell. In one embodiment, the ocular cell-fate further restricted precursor cell comprises a photoreceptor precursor cell. In one embodiment, the ocular cell-fate further restricted precursor cell comprises a retinal ganglion precursor cell. In one embodiment, the ocular cell-fate further restricted precursor cell comprises a retinal pigmented epithelial (RPE) cell. In one embodiment, the ocular cell-fate further restricted precursor cell comprises a corneal endothelial cell. In one embodiment, the ocular cell comprises a cell-fate restricted progeny of ocular stem cell.

Further disclosed herein are methods of treating an ocular disease or disorder in a human subject in need thereof, comprising administering the therapeutic compositions described herein to the human subject.

### BRIEF DESCRIPTION OF THE DRAWINGS

The patent or application file contains at least one drawing executed in color. Copies of this patent or patent application publication with color drawings will be provided by the Office upon request and payment of the necessary fee. The foregoing and other objects, features and advantages will be apparent from the following description of particular embodiments of the disclosure, as illustrated in the accompanying drawings. The drawings are not necessarily to scale, emphasis instead being placed upon illustrating the principles of various embodiments of the disclosure.

FIG. 1 presents the sequence structure for natural α-MSH peptide.

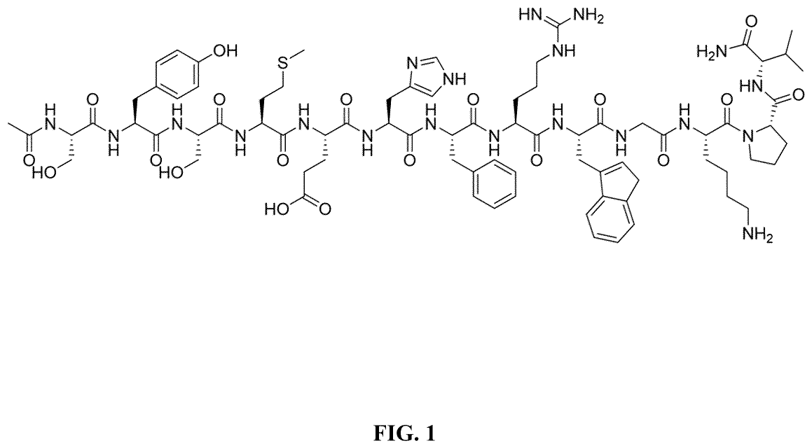

FIG. 2 schematically depicts a diagram of an ocular cell-gene therapy embodiment disclosed herein for the treatment of ocular diseases or disorders by ocular cell-mediated delivery of sustainably expressed α-MSH polypeptide. As depicted, ocular-fate restricted cells can be engineered in vitro to enable them to produce an α-MSH peptide to treat e.g., neovascular ocular diseases or disorders. These engineered ocular cells are formulated to a therapeutic composition, which can then be administered (e.g., injected) into the vitreous (1), the anterior chamber (2), the subretinal space (3), or the suprachoroidal space (4) of a subject's diseased eye. The engineered ocular cells injected to the eye continuously produce and release the α-MSH peptide to the surrounding ocular tissues of an injection site of the eye.

FIG. 3 demonstrates that the α-MSH analog K173 (KR-072) was shown to be delivered to the target tissue, and to have prolonged ocular surface retention and Tₘₐₓ after a single dose. As depicted in FIG. 3, aqueous humor (AH) concentrations were shown to increase with repeated dosing, and measurable levels were detected after 24 hours in all dose groups.

FIGS. 4A, 4B and 4C depict the structures for the α-MSH analog peptides K173 (KR-072), K174 (KR-073) and K150 (KR-049), respectively. The KR-073 and KR-074 α-MSH analog peptides have nearly identical structures and were shown to have very similar ocular distribution patterns. The lack of the HyBA (3,5-hydroxybenzoic acid) residue on the KR-050 α-MSH analog peptide is likely causing the observed difference in corneal and scleral permeability and may increase melanin binding. As depicted, each of the α-MSH peptides analogs demonstrate binding affinities and activities for most MCRs, including MC1-R, MC3-R, MC4-R, and MC5-R affinity.

FIGS. 5A and 5B present the in vivo distribution patterns of certain topically administered α-MSH analog peptides (K173 (KR-072), K174 (KR-073) and K150 (KR-049)) following their administration to minipigs twice daily for four days. As shown in FIGS. 5A and 5B, the topically administered α-MSH analog peptides KR-073 and KR-074 appear to have similar distribution patterns, while KR-050 appears to have decreased scleral/retinal exposure and enhanced melanin binding.

FIG. 6 presents the in vivo distribution patterns of certain topically administered α-MSH analog peptides (KR-073, KR-074 and KR-050) following their administration to minipigs twice daily for four days. As shown in FIG. 6, the α-MSH analog peptides demonstrate enhanced corneal permeability, can be delivered topically and following which they were detected in the central nervous system tissues of the animals, thereby evidencing that such α-MSH analog peptides can be delivered topically for retinal indications.

FIG. 7 depicts normalized reactive oxygen species (ROS) induction data across multiple experimental repeats at 3 hours. As depicted in FIG. 7, both the native α-MSH peptide as well as the α-MSH analog (K174 (KR-073), corresponding to SEQ ID NO: 73) were demonstrated to be protective against ROS accumulation down to the low pM range.

FIG. 8 depicts normalized reactive oxygen species (ROS) induction data across multiple experimental repeats at 4 hours. As depicted in FIG. 8, both the native α-MSH peptide as well as the α-MSH analog (K174 (KR-073), corresponding to SEQ ID NO: 73) were demonstrated to be protective against ROS accumulation down to the low pM range.

FIG. 9 graphically depicts the degree of inhibition of caspase-3 activation following A23187 challenge for ocular stem cells (ABC-123) that were incubated with 100 nM of α-MSH peptide or 100 nM of an α-MSH analog, in each case relative to a control. As shown, the ocular stem cells that were incubated with α-MSH peptide (100 nM) demonstrated a lower percent increase in caspase activation over the control. Similarly, the ocular stem cells that were incubated with α-MSH analog (100 nM) demonstrated a lower percent increase in caspase activation over the control, with the K174 (KR-073) α-MSH analog (SEQ ID NO: 73) demonstrating the lowest percent increase over control. The foregoing therefore evidences that pre-treatment of ocular stem cells with α-MSH and/or an α-MSH analog imparts a beneficial cytoprotective effect to such ocular stem cells.

FIG. 10 graphically depicts the degree of inhibition of caspase-3 activation following A23187 challenge for ocular stem cells (ABC-123) that were incubated with 100 nM of α-MSH peptide or 100 nM of the K174 (KR-073) α-MSH analog (SEQ ID NO: 73), in each case relative to a control. As shown, the ocular stem cells that were incubated with α-MSH peptide (100 nM) demonstrated a lower percent increase in caspase activation over the control, and the ocular stem cells that were incubated with K174 (KR-073) α-MSH analog (SEQ ID NO: 73) demonstrated a lower percent increase in caspase activation over the control and α-MSH analog. The foregoing therefore evidences that pre-treatment of ocular stem cells with an α-MSH analog imparts a higher degree to cytoprotection to such ocular stem cells relative to pre-treatment with α-MSH.

FIG. 11 demonstrates that administration of the evaluated K173 (KR-072) α-MSH analog decreased corneal edema in the rabbit model, thereby evidencing the efficacy of the α-MSH analog. The statistically significant efficacy was observed soon after dosing with 2 lowest treatment arms when looking at corneal stromal thickness by OCT.

FIGS. 12A and 12B show the results of a corneal flat mount analysis performed on 3 animals per group to assess rate of wound closure, and demonstrates that the evaluated K173 (KR-072) α-MSH analog enhanced wound healing in the rabbit model.

### DETAILED DESCRIPTION OF THE DISCLOSURE

Cell transplants are known to be an effective treatment option for a variety of diseases and/or disorders; however, it is not uncommon for the transplanted cells to expire or die after transplant of for the transplanted cells to be rejected by a subject's immune system. Although there are various options available to those of skill in the art to facilitate the transplant, death or rejection of the transplanted cells still regularly occurs. Described herein are agents and compositions that will facilitate and improve the survival of cells or a cell therapy product upon transplant. For example, cells, such as ocular stem cells, co-cultured with an agent described herein may exhibit increased expression of neurotrophic factors, which may increase cell survival upon transplant.

### I. Agents of the Disclosure

### Melanocortin Receptors and α-MSH

### MCR Overview

A family of G-protein-coupled receptors (GPCRs) with seven transmembrane domains have been identified, known as melanocortin receptors (MCRs). Five primary MCR subtypes have been identified in mammals: (1) melanocortin-1 receptors (MC1-R), which are known to be expressed in a range of human cells (including human melanocytes, melanoma cells, CNS gray matter, testis, macrophages, neutrophils, glioma cells, astrocytes, monocytes, and endothelial cells), and are generally associated with melanin formation (e.g., hair pigmentation, skin pigmentation, etc.) and regulating the immune system (e.g., inflammation); (2) melanocortin-2 receptors (MC2-R), which are expressed primarily in adrenal gland cells and are generally associated with steroid-genesis (e.g., corticosteroid production); (3) melanocortin-3 receptors (MC3-R), which are expressed primarily in CNS cells (such as in the hypothalamus, mid-brain and brainstem) and are generally associated with energy homeostasis, food intake, and inflammation; (4) melanocortin-4 receptors (MC4-R), which are also expressed primarily in CNS cells and are generally associated with feeding behavior, energy homeostasis, and sexual function; and (5) melanocortin-5 receptors (MC5-R), which are broadly expressed in various peripheral tissues and are generally associated with regulation of the exocrine gland system.

MCRs and corresponding melanocortic peptides have also been found to mediate a number of other physiological conditions, including: immunomodulation, motivation, learning, memory, behaviour, inflammation, body temperature, pain, perception, blood pressure, heart rate, vascular tone, brain blood flow, nerve growth, placental development, aldosteron synthesis and release, thyroxin release, spermatogenesis, ovarian weight, prolactin and FSH secretion, uterine bleeding in women, sebum and pheromone secretion, blood glucose levels, weight homeostasis, and intrauterine fetal growth (as well as other events surrounding parturition).

Melanocortins are a family of regulatory peptides known to have agonistic and antagonistic binding affinities to MCRs. Natural melanocortins are synthesized by post-translational processing of the hormone proopiomelanocortin (POMC-131 amino acids long).

An exemplary amino acid sequence of the human POMC preprotein (NCBI Accession No. NP_001306134.1 and UniProt Accession No. P01189) is as follows:

(SEQ ID NO: 80)
MPRSCCSRSGALLLALLLQASMEVRGWCLESSQCQDLTTESNLLECIRA
CKPDLSAETPMFPGNGDEQPLTENPRKYVMGHFRWDRFGRRNSSSSGSS
GAGQKREDVSAGEDCPLPEGGPEPRSDGAKPGPREGKRSYSMEHFRWGK
PVGKKRRPVKVYPNGAEDESAEAFPLEFKRELTGQRLREGDGPDGPADD
GAGAQADLEHSLLVAAEKKDEGPYRMEHFRWGSPPKDKRYGGFMTSEKS
QTPLVTLFKNAIIKNAYKKGE.

In the POMC amino acid sequence above, positions 1 to 26 correspond to the signal peptide; positions 27 to 102 correspond to the N-terminal peptide of pro-opiomelanocortin (NPP); positions 77 to 87 correspond to γ-MSH; positions 105 to 134 correspond to a potential peptide; positions 138 to 176 correspond to corticotropin; positions 138 to 150 correspond to α-MSH; positions 156 to 176 correspond to corticotropin-like intermediary peptide; positions 179 to 267 correspond to lipotropin beta; positions 179 to 234 correspond to lipotropin gamma; positions 217-234 correspond to β-MSH; positions 237 to 267 correspond to β-endorphin; and positions 237 to 241 correspond to met-enkephalin.

The post-translational processing of POMC produces three primary classes of hormones: melanocortins (MCs), adrenocorticotropin (ACTH), and certain endorphins (e.g., lignotropin). Melanocortins (such as α-MSH, β-MSH, γ-MSH) are known to have a range of binding affinities and activities for most MCRs, including MC1-R, MC3-R, MC4-R, and MC5-R. Binding to MC2-R is generally limited to adrenocorticotropin (ACTH).

MCRs are viewed as promising therapeutic targets for treating a range of major pathologies and indications, including obesity, diabetes, inflammatory conditions, and sexual dysfunction. However, many melanocortins (and analogs thereof) have limited selectivity between various MCRs and tissue types, with few MCR-specific peptides having been identified as effective therapeutics or approved for pharmaceutical applications. Likewise, the modelling and design of highly selective MCR agonists/antagonists for specific receptor subtypes in specific tissues for specific indications remains a difficult task. A strong need therefore exists for novel MCR-targeting compounds (e.g., novel α-MSH peptide analogs) which are highly specific to particular MCR subtypes, particular tissues, and/or particular therapeutic indications.

In some embodiments, compounds of the present disclosure interact with one or more MCRs to affect their activity (e.g., agonist, antagonist, etc.).

### α-MSH Analogs

The present disclosure presents novel α-MSH peptide analogs. In some embodiments, the novel α-MSH peptide analog is a peptidomimetic of natural α-MSH peptide. In some embodiments, the α-MSH analog contains structural elements that are not found in natural peptides (i.e., peptides comprised of only the 20 proteinogenic amino acids). In some embodiments, the α-MSH peptidomimetic analog contains changes in structural sequence (additions, deletions, substitutions) and/or the presence of amino acids that do not occur in nature, as compared to natural α-MSH peptide. In some embodiments, the α-MSH analog contains one or more fragments of natural α-MSH peptide. In some embodiments, the α-MSH analog can contain one or more of the following: amino acids with side chains that are not found among the known 20 proteinogenic amino acids; non-peptide-based bridging moieties (e.g., cyclic structure, link, staple, bridge, etc.) used to effect cyclization (i.e., conformational constraints) between the ends or internal portions of the molecule; substitutions of the amide bond hydrogen moiety by methyl groups (N-methylation) or other alkyl groups; stereoisomerization between L- and D-amino acids; replacement of a peptide bond with a chemical group or bond that is resistant to chemical or enzymatic treatments; N- and C-terminal modifications; conjugation with a non-peptidic (i.e., non-peptide-based) extensions (such as polyethylene glycol, lipids, carbohydrates, nucleosides, nucleotides, nucleoside bases, various small molecules, or phosphate or sulfate groups); or combinations thereof.

α-MSH is also known as alpha-melanotropin, alpha-melanocortin, and alpha-intermedin. α-MSH is a linear tridecapeptide melanocortin having the following formula: Ac-Ser-Tyr-Ser-Met-Glu-His-Phe-Arg-Trp-Gly-Lys-Pro-Val-NH2 (SEQ ID NO: 76) (IUPAC name: N-acetyl-L-seryl-L-tyrosyl-L-seryl-L-methionyl-L-α-glutamyl-L-histidyl-L-phenylalanyl-L-arginyl-L-tryptophylglycyl-L-lysyl-L-prolyl-L-valinamide (SEQ ID NO: 76)).

Natural alpha-melanocyte stimulating hormone (α-MSH) is as a cleavage product derived from the large precursor protein, proopiomelanocortin (POMC). α-MSH is an endogenous ligand to the melanocortin receptor MC1 (MC1-R), with α-MSH binding to MC1-R at a sub-nanomolar binding affinity. α-MSH also binds to other melanocortin receptors, including MC3-R, MC4-R, and MC5-R; α-MSH does not bind to MC2-R.

Studies of α-MSH, as well as various α-MSH analogs, have shown the heptapeptide sequence of Met⁴-Glu⁵-His⁶-Phe⁷-Arg⁸-Trp⁹-Gly¹⁰ (SEQ ID NO: 81) (i.e., Methionine-Glutamic acid-Histidine-Phenylalanine-Arginine-Tryptophan-Glycine (SEQ ID NO: 81)) to function as a common active core (CAC) within α-MSH-derived peptides. Similar studies of α-MSH analogs have shown the quadrapeptide sequence of His⁶-Phe⁷-Arg⁸-Trp⁹ (SEQ ID NO: 82) (i.e., Histidine-Phenylalanine-Arginine-Tryptophan (SEQ ID NO: 82)) to generally function as the central active core for MC1-R activity.

In some embodiments, the present disclosure presents novel α-MSH peptide analogs that bind with high affinity to one or more melanocortin receptors (MCRs) (e.g., MC1-R). In some embodiments, the present disclosure presents novel α-MSH peptide analogs that bind with high affinity to MC1-R. In some embodiments, the α-MSH analog is an MC1-R preferring ligand. A ligand can be “MC1-R-preferring” in two ways: (1) A high binding preference to MC1-R (e.g., lower EC₅₀ for MC1-R than other MCRs), and/or (2) A higher binding strength to MC1-R. In some embodiments, the α-MSH analog is an MC1-R preferring ligand, and has an EC₅₀ for MC1-R of less than 5%, 10%, 20%, 50% or 100% compared to the EC₅₀ for one or more other MCRs (e.g., as measured by a cAMP assay). In some embodiments, the α-MSH analog is an MC1-R preferring ligand, and has an EC₅₀ for MC1-R of less than 5%, 10%, 20%, 50% or 100% compared to the EC₅₀ for MC2-R, MC3-R, MC4-R, and/or MC5-R (e.g., as measured by a cAMP assay). In some embodiments, the α-MSH analog is an MC1-R preferring ligand, and has an IC₅₀ for MC1-R of less than 5%, 10%, 20%, 50% or 100% compared to the IC₅₀ for one or more other MCRs (e.g., as measured by a competitive binding assay). In some embodiments, the α-MSH analog is an MC1-R preferring ligand, and has an IC₅₀ for MC1-R of less than 5%, 10%, 20%, 50% or 100% compared to the IC₅₀ for MC2-R, MC3-R, MC4-R, and/or MC5-R (e.g., as measured by a competitive assay).

In some embodiments, the present disclosure presents novel α-MSH peptide analogs that bind with high affinity to MC3-R. In some embodiments, the α-MSH analog is an MC3-R preferring ligand. A ligand can be “MC3-R-preferring” in two ways: (1) A high binding preference to MC3-R (e.g., lower EC₅₀ for MC3-R than other MCRs), and/or (2) A higher binding strength to MC3-R. In some embodiments, the α-MSH analog is an MC3-R preferring ligand, and has an EC₅₀ for MC3-R of less than 5%, 10%, 20%, 50% or 100% compared to the EC₅₀ for one or more other MCRs (e.g., as measured by a cAMP assay). In some embodiments, the α-MSH analog is an MC3-R preferring ligand, and has an EC₅₀ for MC3-R of less than 5%, 10%, 20%, 50% or 100% compared to the EC₅₀ for MC1-R, MC2-R, MC4-R, and/or MC5-R (e.g., as measured by a cAMP assay). In some embodiments, the α-MSH analog is an MC3-R preferring ligand, and has an IC₅₀ for MC3-R of less than 5%, 10%, 20%, 50% or 100% compared to the IC₅₀ for one or more other MCRs (e.g., as measured by a competitive binding assay). In some embodiments, the α-MSH analog is an MC3-R preferring ligand, and has an IC₅₀ for MC3-R of less than 5%, 10%, 20%, 50% or 100% compared to the IC₅₀ for MC1-R, MC2-R, MC4-R, and/or MC5-R (e.g., as measured by a competitive assay).

In some embodiments, the present disclosure presents novel α-MSH peptide analogs that bind with high affinity to MC4-R. In some embodiments, the α-MSH analog is an MC4-R preferring ligand. A ligand can be “MC4-R-preferring” in two ways: (1) A high binding preference to MC4-R (e.g., lower EC₅₀ for MC4-R than other MCRs), and/or (2) A higher binding strength to MC4-R. In some embodiments, the α-MSH analog is an MC4-R preferring ligand, and has an EC₅₀ for MC4-R of less than 5%, 10%, 20%, 50% or 100% compared to the EC₅₀ for one or more other MCRs (e.g., as measured by a cAMP assay). In some embodiments, the α-MSH analog is an MC4-R preferring ligand, and has an EC₅₀ for MC4-R of less than 5%, 10%, 20%, 50% or 100% compared to the EC₅₀ for MC1-R, MC2-R, MC3-R, and/or MC5-R (e.g., as measured by a cAMP assay). In some embodiments, the α-MSH analog is an MC4-R preferring ligand, and has an IC₅₀ for MC4-R of less than 5%, 10%, 20%, 50% or 100% compared to the IC₅₀ for one or more other MCRs (e.g., as measured by a competitive binding assay). In some embodiments, the α-MSH analog is an MC4-R preferring ligand, and has an IC₅₀ for MC4-R of less than 5%, 10%, 20%, 50% or 100% compared to the IC₅₀ for MC1-R, MC2-R, MC3-R, and/or MC5-R (e.g., as measured by a competitive assay).

In some embodiments, the present disclosure presents novel α-MSH peptide analogs that bind with high affinity to MC5-R. In some embodiments, the α-MSH analog is an MC5-R preferring ligand. A ligand can be “MC5-R-preferring” in two ways: (1) A high binding preference to MC5-R (e.g., lower EC₅₀ for MC5-R than other MCRs), and/or (2) A higher binding strength to MC5-R. In some embodiments, the α-MSH analog is an MC5-R preferring ligand, and has an EC₅₀ for MC5-R of less than 5%, 10%, 20%, 50% or 100% compared to the EC₅₀ for one or more other MCRs (e.g., as measured by a cAMP assay). In some embodiments, the α-MSH analog is an MC5-R preferring ligand, and has an EC₅₀ for MC5-R of less than 5%, 10%, 20%, 50% or 100% compared to the EC₅₀ for MC1-R, MC2-R, MC3-R, and/or MC4-R (e.g., as measured by a cAMP assay). In some embodiments, the α-MSH analog is an MC5-R preferring ligand, and has an IC₅₀ for MC5-R of less than 5%, 10%, 20%, 50% or 100% compared to the IC₅₀ for one or more other MCRs (e.g., as measured by a competitive binding assay). In some embodiments, the α-MSH analog is an MC5-R preferring ligand, and has an IC₅₀ for MC5-R of less than 5%, 10%, 20%, 50% or 100% compared to the IC₅₀ for MC1-R, MC2-R, MC3-R, and/or MC4-R (e.g., as measured by a competitive assay).

In some embodiments, the present disclosure presents novel α-MSH peptide analogs that bind with high affinity to one or more of MC1-R, MC3-R, MC4-R, MC5-R, or a combination thereof.

In some embodiments, the α-MSH peptide analog binds with high affinity to one or more corneal MCRs (e.g., corneal MC1-R), and has sufficient active properties (e.g., agonist properties, ADME, pK) to be suitable for use in the treatment of corneal endothelial cell loss related diseases. In some embodiments, the α-MSH peptide analog binds with high affinity to corneal MC1-R, and has sufficient active properties (e.g., agonist properties, ADME, pK) to be suitable for use in the treatment of corneal endothelial cell loss related diseases.

In some embodiments, the α-MSH peptide analog binds with high affinity to one or more MCRs found in the ocular tissues, and has sufficient active properties (e.g., agonist properties, ADME, pK) to be suitable for use in the treatment of ocular disorders or diseases. In some embodiments, the α-MSH peptide analog binds with high affinity to one or more corneal MCRs, and has sufficient active properties (e.g., agonist properties, ADME, pK) to be suitable for use in the treatment of corneal epithelial cell loss related diseases. In some embodiments, the α-MSH peptide analog binds with high affinity to one or more retinal MCRs, and has sufficient active properties (e.g., agonist properties, ADME, pK) to be suitable for use in the treatment of retinal cell loss related diseases.

In some embodiments, α-MSH analogs of the present disclosure comprise one or more modifications (e.g., substitution, addition, deletion) to the peptide sequence of natural α-MSH. In some embodiments, α-MSH analogs of the present disclosure can comprise one or more modifications (e.g., substitution, addition, deletion) to the peptide sequence of natural α-MSH, as described in U.S. Pat. No. 4,457,864. U.S. Pat. Nos. 4,485,039, 5,683,981, 5,714,576, 5,731,408, 6,051,555, 6,054,556, 6,284,735, 6,350,430, 6,476,187, 6,534,503, 6,579,968, 6,600,015, 6,693,165, 6,699,873, 6,887,846, 6,960,646, 6,951,916, 7,045,591, 7,049,398, 7,084,111, 7,307,063, 7,342,089, 7,345,144, 7,368,433, 7,385,025, 7,417,027, 7,517,854, 7,550,602, 7,582,610, 7,601,753, 7,662,782, 7,897,721, 7,795,378, 8,247,530, 8,349,797, 8,703,702, 9,040,663, 9,273,098, 9,850,280, 9,951,116, 10,106,578, 10,238,758, 10,632,171, 10,660,939, 10,711,039, 10,857,208, 20020143141, US20030064921, US20050130901, US20050187164, US20060105951, U.S. Pat. No. 20060122121, US20060293223, US20070027091, US20070105759, US20070123453, U.S. Pat. No. 20070155660, US20070244054, US20080039387, US20080177036, US20080207493, U.S. Pat. No. 20080280820, US20080306008, US20090203581, US20090305960, US20110098213, U.S. Pat. No. 20140050773, US20150297672, WO 1998027113, WO 1999054358, WO 2000005263, WO 2001030808, WO 2001085930, WO 2001090140, WO 2002026774, WO 2005079574, WO 2009006141, WO 2009144432, WO 2009144433, WO 2010065799, WO 2010065800, WO 2010065801, WO 2010065802, WO 2019183472, and WO 2020060983; the contents of which are each incorporated herein by reference in their entirety, as related to α-MSH analogs, and synthesis, use, and formulations thereof.

### Fragments and Substitutions

In some embodiments, α-MSH analogs of the present disclosure can comprise one or more fragments of the peptide sequence of natural α-MSH. In some embodiments, α-MSH analogs of the present disclosure can comprise a deletional variant of natural α-MSH. In some embodiments, α-MSH analogs of the present disclosure can comprise a truncated variant of natural α-MSH. In some embodiments, α-MSH analogs of the present disclosure can comprise a deletional and truncated variant of natural α-MSH. In some embodiments, α-MSH analogs of the present disclosure can comprise one or more fragments of the peptide sequence of natural α-MSH, as shown in Table 1.

TABLE 1
α-MSH analogs - fragments

| | | | | | | | | | | | | | | C- | SEQ |
|---|---|---|---|---|---|---|---|---|---|---|---|---|---|---|---|
| N-term | AA1 | AA2 | AA3 | AA4 | AA5 | AA6 | AA7 | AA8 | AA9 | AA10 | AA11 | AA12 | AA13 | term | ID NO: |
| Ac | Ser | Tyr | Ser | Met | Glu | His | Phe | Arg | Trp | Gly | Lys | Pro | Val | NH2 | 76 |
| Ac | Ser | Tyr | Ser | Met | Glu | His | Phe | Arg | Trp | Gly | Lys | Pro | | NH2 | 5 |
| Ac | Ser | Tyr | Ser | Met | Glu | His | Phe | Arg | Trp | Gly | Lys | | | NH2 | 6 |
| Ac | Ser | Tyr | Ser | Met | Glu | His | Phe | Arg | Trp | Gly | | | | NH2 | 83 |
| Ac | Ser | Tyr | Ser | Met | Glu | His | Phe | Arg | Trp | | | | | NH2 | 84 |
| Ac | | Tyr | Ser | Met | Glu | His | Phe | Arg | Trp | Gly | Lys | Pro | Val | NH2 | 1 |
| Ac | | Tyr | Ser | Met | Glu | His | Phe | Arg | Trp | Gly | Lys | Pro | | NH2 | 85 |
| Ac | | Tyr | Ser | Met | Glu | His | Phe | Arg | Trp | Gly | Lys | | | NH2 | 86 |
| Ac | | Tyr | Ser | Met | Glu | His | Phe | Arg | Trp | Gly | | | | NH2 | 87 |
| Ac | | Tyr | Ser | Met | Glu | His | Phe | Arg | Trp | | | | | NH2 | 88 |
| Ac | | | Ser | Met | Glu | His | Phe | Arg | Trp | Gly | Lys | Pro | Val | NH2 | 2 |
| Ac | | | Ser | Met | Glu | His | Phe | Arg | Trp | Gly | Lys | Pro | | NH2 | 89 |
| Ac | | | Ser | Met | Glu | His | Phe | Arg | Trp | Gly | Lys | | | NH2 | 90 |
| Ac | | | Ser | Met | Glu | His | Phe | Arg | Trp | Gly | | | | NH2 | 91 |
| Ac | | | Ser | Met | Glu | His | Phe | Arg | Trp | | | | | NH2 | 92 |
| Ac | | | | Met | Glu | His | Phe | Arg | Trp | Gly | Lys | Pro | Val | NH2 | 3 |
| Ac | | | | Met | Glu | His | Phe | Arg | Trp | Gly | Lys | Pro | | NH2 | 93 |
| Ac | | | | Met | Glu | His | Phe | Arg | Trp | Gly | Lys | | | NH2 | 94 |
| Ac | | | | Met | Glu | His | Phe | Arg | Trp | Gly | | | | NH2 | 95 |
| Ac | | | | Met | Glu | His | Phe | Arg | Trp | | | | | NH2 | 96 |
| Ac | | | | | Glu | His | Phe | Arg | Trp | Gly | Lys | Pro | Val | NH2 | 4 |
| Ac | | | | | Glu | His | Phe | Arg | Trp | Gly | Lys | Pro | | NH2 | 7 |
| Ac | | | | | Glu | His | Phe | Arg | Trp | Gly | Lys | | | NH2 | 97 |
| Ac | | | | | Glu | His | Phe | Arg | Trp | Gly | | | | NH2 | 98 |
| Ac | | | | | Glu | His | Phe | Arg | Trp | | | | | NH2 | 99 |
| Ac | | | | | | His | Phe | Arg | Trp | Gly | Lys | Pro | Val | NH2 | 100 |
| Ac | | | | | | His | Phe | Arg | Trp | Gly | Lys | Pro | | NH2 | 101 |
| Ac | | | | | | His | Phe | Arg | Trp | Gly | Lys | | | NH2 | 102 |
| Ac | | | | | | His | Phe | Arg | Trp | Gly | | | | NH2 | 103 |
| Ac | | | | | | His | Phe | Arg | Trp | | | | | NH2 | 104 |

### Ser¹

In some embodiments, α-MSH analogs of the present disclosure can comprise one or more modifications (e.g., substitution, addition, deletion) to the Ser¹ amino acid of the peptide sequence, as compared to natural α-MSH (or fragments thereof, e.g., as in Table 1). In some embodiments, α-MSH analogs of the present disclosure can comprise a deletion of the Ser¹ amino acid of the peptide sequence, as compared to natural α-MSH (or fragments thereof, e.g., as in Table 1). In some embodiments, α-MSH analogs of the present disclosure can comprise a substitution of the Ser¹ amino acid with one or more of the following moieties: D-Ser, NMe-Ser, Ile, Thr, Tyr, or Tyr(Me), or D-stereoisomers thereof.

### Tyr²

In some embodiments, α-MSH analogs of the present disclosure can comprise one or more modifications (e.g., substitution, addition, deletion) to the Tyr² amino acid of the peptide sequence, as compared to natural α-MSH (or fragments thereof, e.g., as in Table 1). In some embodiments, α-MSH analogs of the present disclosure can comprise a deletion of the Tyr² amino acid of the peptide sequence, as compared to natural α-MSH (or fragments thereof, e.g., as in Table 1). In some embodiments, α-MSH analogs of the present disclosure can comprise a deletion of the Ser¹ and Tyr² amino acids of the peptide sequence, as compared to natural α-MSH (or fragments thereof, e.g., as in Table 1). In some embodiments, α-MSH analogs of the present disclosure can comprise a substitution of the Tyr² amino acid with one or more of the following moieties: D-Tyr, Ile, 1-Nal, 2-Nal, 2-Pal, 3-Pal, D-Phe, 3Cl-Phe, 4F-Phe, 4Cl-Phe, D-Phe(2,4-DiCl), Ser, Thr, Tic, or Tyr(Me), or D-stereoisomers thereof. In some embodiments, α-MSH analogs of the present disclosure can comprise a substitution of the Tyr² amino acid with a Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof.

### Ser³

In some embodiments, α-MSH analogs of the present disclosure can comprise one or more modifications (e.g., substitution, addition, deletion) to the Ser³ amino acid of the peptide sequence, as compared to natural α-MSH (or fragments thereof, e.g., as in Table 1). In some embodiments, α-MSH analogs of the present disclosure can comprise a deletion of the Ser³ amino acid of the peptide sequence, as compared to natural α-MSH (or fragments thereof, e.g., as in Table 1). In some embodiments, α-MSH analogs of the present disclosure can comprise a deletion of the Ser¹. Tyr², and Ser³ amino acids of the peptide sequence, as compared to natural α-MSH (or fragments thereof, e.g., as in Table 1). In some embodiments, α-MSH analogs of the present disclosure can comprise a substitution of the Ser³ amino acid with one or more of the following moieties: BrAc, D-Ser, Ile, Leu, Nle, Tyr, or Val, or D-stereoisomers thereof and optionally forms a cyclic peptide with suitable chemical groups at the 9, 10, 11 or 12 position.

### Met⁴

In some embodiments, α-MSH analogs of the present disclosure can comprise one or more modifications (e.g., substitution, addition, deletion) to the Met⁴ amino acid of the peptide sequence, as compared to natural α-MSH (or fragments thereof, e.g., as in Table 1). In some embodiments, α-MSH analogs of the present disclosure can comprise a deletion of the Met⁴ amino acid of the peptide sequence, as compared to natural α-MSH (or fragments thereof, e.g., as in Table 1). In some embodiments, α-MSH analogs of the present disclosure can comprise a deletion of the Ser¹, Tyr², Ser³, and Met⁴ amino acids of the peptide sequence, as compared to natural α-MSH (or fragments thereof, e.g., as in Table 1). In some embodiments, α-MSH analogs of the present disclosure can comprise a substitution of the Met⁴ amino acid with one or more of the following moieties: Lys, Lys(N3), BrAc, R4-R10, S4-S10, D-Met, Asp, Can, Cha, Cha, Cpna, Cpra, Cys, D-Cys, hCys, D-hCys, Glu, Gly, Hcy, Hle, Ile, Leu, (cyclohexyl)Gly, Nle, Nle(Met), Pen, D-Pen, Ser, Tyr, or Val, or D-stereoisomers thereof and optionally forms a cyclic peptide with suitable chemical groups at the 9, 10, 11 or 12 position.

### Glu⁵

In some embodiments, α-MSH analogs of the present disclosure can comprise one or more modifications (e.g., substitution, addition, deletion) to the Glu⁵ amino acid of the peptide sequence, as compared to natural α-MSH (or fragments thereof, e.g., as in Table 1). In some embodiments, α-MSH analogs of the present disclosure can comprise a deletion of the Glu⁵ amino acid of the peptide sequence, as compared to natural α-MSH (or fragments thereof, e.g., as in Table 1). In some embodiments, α-MSH analogs of the present disclosure can comprise a deletion of the Ser¹, Tyr², Ser³, Met⁴, and Glu⁵ amino acids of the peptide sequence, as compared to natural α-MSH (or fragments thereof, e.g., as in Table 1). In some embodiments, α-MSH analogs of the present disclosure can comprise a substitution of the Glu⁵ amino acid with one or more of the following moieties: D-Glu, Ala, Asn, Asp, Cys, D-Cys, hCys, α-Me-Cys, Dab, NDab, Dap, hGlu, Gln, Gly, Ile, Lys, D-Lys, Lys(N3), BrAc, R4-R10, S4-S10, NGlu, DabN3, Pra, OrnN3, Nle, Orn, Ser, Succ, Tyr, or 4-aminobutyric acid, or D-stereoisomers thereof and optionally forms a cyclic peptide with suitable chemical groups at the 9, 10, 11 or 12 position.

### His⁶

In some embodiments, α-MSH analogs of the present disclosure can comprise one or more modifications (e.g., substitution, addition, deletion) to the His⁶ amino acid of the peptide sequence, as compared to natural α-MSH (or fragments thereof, e.g., as in Table 1). In some embodiments, α-MSH analogs of the present disclosure can comprise a substitution of the His⁶ amino acid with one or more of the following moieties: D-His, NMe-His, NMe-D-His, Phe-4-NH2, Aib, Aic, Ala, D-Ala, Arg, Asn, Asp, Cha, Chg, Cit, Cys, D-Cys, Dab, Dap, Gln, Glu, His(1-Me), His(3-Me), Hyp, Hyp(Bzl), Ile, Leu, Lys, Met, Met(O), Met(O₂), Nle, D-Nle, Orn, 2-Pal, 3-Pal, 4-Pal, Phe, Pro, Sar, Ser, Ser(Bzl), Thr, Thr(OBzl), Tic, Tle, Trp, Tyr, Tyr(Me), or Val, or D-stereoisomers thereof and optionally forms a cyclic peptide with suitable chemical groups at the 9, 10, 11 or 12 position.

In some embodiments, α-MSH analogs of the present disclosure can comprise a substitution of the His⁶ amino acid with one or more of the following moieties: NMe-His, NMe-D-His, Phe-4—NH₂. Tyr(Me), cyclohexylglycine, cyclohexylalanine, tert-butylglycine, Gln(alkyl), Gln(aryl), Asn(alkyl), Asn(aryl), Tic, (2-pyridinyl)alanine, (3-pyridinyl)alanine, (4-pyridinyl)alanine, (2-thienyl)alanine, (3-thienyl)alanine, (4-thiazolyl)Ala, (2-furyl)alanine, or (3-furyl)alanine. In some embodiments, α-MSH analogs of the present disclosure can comprise a substitution of the His⁶ amino acid with a Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof.

### Phe⁷

In some embodiments, α-MSH analogs of the present disclosure can comprise one or more modifications (e.g., substitution, addition, deletion) to the Phe⁷ amino acid of the peptide sequence, as compared to natural α-MSH (or fragments thereof, e.g., as in Table 1). In some embodiments, α-MSH analogs of the present disclosure can comprise a substitution of the Phe⁷ amino acid with one or more of the following moieties: D-Phe, NMe-Phe, D-NMe-Phe, D-hPhe, Arg, D-Arg, Bip, D-Bip, Cys, D-Cys, Dip, D-Dip, His, 1-Nal, D-1-Nal, 2-Nal, D-2-Nal, 2-Pal, 3-Pal, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3-CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Tic, Thi, D-Thi, Trp, Tyr, D-Tyr, Tyr(Me), D-Tyr(Me), Val, or D-Val, or D-stereoisomers thereof. In some embodiments, α-MSH analogs of the present disclosure can comprise a substitution of the Phe⁷ amino acid with a D-Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof.

### Arg⁸

In some embodiments, α-MSH analogs of the present disclosure can comprise one or more modifications (e.g., substitution, addition, deletion) to the Arg⁸ amino acid of the peptide sequence, as compared to natural α-MSH (or fragments thereof, e.g., as in Table 1). In some embodiments, α-MSH analogs of the present disclosure can comprise a substitution of the Arg⁸ amino acid with one or more of the following moieties: hArg, norArg, D-Arg, Arg (Me), Arg (Me)₂, Ala, Cys, D-Cys, Dab, Dap, Dpr (beta-Ala), Leu, Lys, hLys, Nle, (Nlys)Gly, NMe-Arg, Orn, Phe, Phe(4-Cl), D-Phe(4-Cl), Ser, or Trp, or D-stereoisomers thereof In some embodiments, α-MSH analogs of the present disclosure can comprise a substitution of the Arg⁸ amino acid with a Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof.

### Trp⁹

In some embodiments, α-MSH analogs of the present disclosure can comprise one or more modifications (e.g., substitution, addition, deletion) to the Trp⁹ amino acid of the peptide sequence, as compared to natural α-MSH (or fragments thereof, e.g., as in Table 1). In some embodiments, α-MSH analogs of the present disclosure can comprise a substitution of the Trp⁹ amino acid with one or more of the following moieties: D-Trp, Trp(6-Me), Trp(7-Me), Aic, Atc. Ala, Arg, Asp, Bip, Cys, D-Cys, Cys-Trp, α-Me-Cys, Dab, 1-Nal, D-1-Nal, 2-Nal, D-2-Nal, NMe-Trp, Trp(Me), Tic, D-Tic, Tiq, D-Tiq. Tpi, or D-Tpi, R4-R10, S4-S10, Ndab, Orn, or D-stereoisomers thereof. In some embodiments, α-MSH analogs of the present disclosure can comprise a substitution of the Trp⁹ amino acid with a Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof and optionally forms a cyclic peptide with suitable chemical groups at the 3, 4 or 5 position.

### Gly¹⁰

In some embodiments, α-MSH analogs of the present disclosure can comprise one or more modifications (e.g., substitution, addition, deletion) to the Gly¹⁰ amino acid of the peptide sequence, as compared to natural α-MSH (or fragments thereof, e.g., as in Table 1). In some embodiments, α-MSH analogs of the present disclosure can comprise a deletion of the Gly¹⁰ amino acid of the peptide sequence, as compared to natural α-MSH (or fragments thereof, e.g., as in Table 1). In some embodiments, α-MSH analogs of the present disclosure can comprise a deletion of the Gly¹⁰, Lys¹¹, Pro¹², and Val¹³ amino acids of the peptide sequence, as compared to natural α-MSH (or fragments thereof, e.g., as in Table 1). In some embodiments, α-MSH analogs of the present disclosure can comprise a substitution of the Gly¹⁰ amino acid with one or more of the following moieties: D-Gly, Ala, D-Ala, Arg, 6-Ahx, Cys, D-Cys, Daa, Dab, DabN3, Glu, Lys, D-Lys, hLys, LysN3, Orn, Pra, Hpra, Trp, R4-R10, S4-S10, or 5-aminopentanoic acid, or D-stereoisomers thereof and optionally forms a cyclic peptide with suitable chemical groups at the 3, 4 or 5 position.

### Lys¹¹

In some embodiments, α-MSH analogs of the present disclosure can comprise one or more modifications (e.g., substitution, addition, deletion) to the Lys¹¹ amino acid of the peptide sequence, as compared to natural α-MSH (or fragments thereof, e.g., as in Table 1). In some embodiments, α-MSH analogs of the present disclosure can comprise a deletion of the Lys¹¹ amino acid of the peptide sequence, as compared to natural α-MSH (or fragments thereof, e.g., as in Table 1). In some embodiments, α-MSH analogs of the present disclosure can comprise a deletion of the Lys¹¹, Pro¹², and Val¹³ amino acids of the peptide sequence, as compared to natural α-MSH (or fragments thereof, e.g., as in Table 1). In some embodiments, α-MSH analogs of the present disclosure can comprise a substitution of the Lys¹¹ amino acid with one or more of the following moieties: D-Lys, Ala, Asn, Asp, Cys, D-Cys, Dab, Glu, hGlu, Hgin, Gly, Lys(Me)₂, Pra, or Orn, or D-stereoisomers thereof and optionally forms a cyclic peptide with suitable chemical groups at the 3, 4 or 5 position.

### Pro¹²

In some embodiments, α-MSH analogs of the present disclosure can comprise one or more modifications (e.g., substitution, addition, deletion) to the Pro¹² amino acid of the peptide sequence, as compared to natural α-MSH (or fragments thereof, e.g., as in Table 1). In some embodiments, α-MSH analogs of the present disclosure can comprise a deletion of the Pro¹² amino acid of the peptide sequence, as compared to natural α-MSH (or fragments thereof, e.g., as in Table 1). In some embodiments, α-MSH analogs of the present disclosure can comprise a deletion of the Pro¹², and Val¹³ amino acids of the peptide sequence, as compared to natural α-MSH (or fragments thereof, e.g., as in Table 1). In some embodiments, α-MSH analogs of the present disclosure can comprise a substitution of the Pro¹² amino acid with one or more of the following moieties: D-Pro, Ala, D-Ala, Asp, Cys, D-Cys, Gly, D-Gly, Ile, D-Ile, Leu, D-Leu, Met, D-Met, Phe, D-Phe, Ser, Trp, D-Trp, Val, or D-Val, or D-stereoisomers thereof and optionally forms a cyclic peptide with suitable chemical groups at the 3, 4 or 5 position. In some embodiments, α-MSH analogs of the present disclosure can comprise a substitution of the Pro¹² amino acid with a Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof.

### Val¹³

In some embodiments, α-MSH analogs of the present disclosure can comprise one or more modifications (e.g., substitution, addition, deletion) to the Val¹³ amino acid of the peptide sequence, as compared to natural α-MSH (or fragments thereof, e.g., as in Table 1). In some embodiments, α-MSH analogs of the present disclosure can comprise a deletion of the Val¹³ amino acid of the peptide sequence, as compared to natural α-MSH (or fragments thereof, e.g., as in Table 1). In some embodiments, α-MSH analogs of the present disclosure can comprise a substitution of the Val¹³ amino acid with one or more of the following moieties: D-Val, Nme-Val, Ala, D-Ala, Can, Cba, Cha, Cpna, Cpra, Cys, D-Cys, Hcy, Hle, Ile, D-Ile, Leu, D-Leu, Met, D-Met, Nle, Pro, or D-Pro, or D-stereoisomers thereof.

### Terminal Modifications and Conjugations

In some embodiments, α-MSH analogs of the present disclosure can comprise one or more modifications (e.g., substitution, addition, deletion) to one or more terminus (e.g., N-terminus, C-terminus, or both) of the peptide sequence, as compared to natural α-MSH (or fragments thereof, e.g., as in Table 1). In some embodiments, α-MSH analogs of the present disclosure can comprise one or more modifications (e.g., substitution, addition, deletion) to the N-terminus of the peptide sequence, as compared to natural α-MSH (or fragments thereof, e.g., as in Table 1). In some embodiments, α-MSH analogs of the present disclosure can comprise one or more modifications (e.g., substitution, addition, deletion) to the C-terminus of the peptide sequence, as compared to natural α-MSH (or fragments thereof, e.g., as in Table 1). In some embodiments, α-MSH analogs of the present disclosure can comprise one or more modifications (e.g., substitution, addition, deletion) to both the N-terminus of the peptide sequence and the C-terminus of the peptide sequence, as compared to natural α-MSH (or fragments thereof, e.g., as in Table 1).

In some embodiments, α-MSH analogs of the present disclosure can comprise modifications to the N-terminus of the peptide sequence with one or more peptide-based moieties. In some embodiments, α-MSH analogs of the present disclosure can comprise modifications to the C-terminus of the peptide sequence with one or more peptide-based moieties. In some embodiments, α-MSH analogs of the present disclosure can comprise modifications to both the N-terminus of the peptide sequence and the C-terminus of the peptide sequence with one or more peptide-based moieties.

In some embodiments, α-MSH analogs of the present disclosure can comprise modifications to the N-terminus of the peptide sequence with one or more non-peptide-based moieties. In some embodiments, α-MSH analogs of the present disclosure can comprise modifications to the C-terminus of the peptide sequence with one or more non-peptide-based moieties. In some embodiments, α-MSH analogs of the present disclosure can comprise modifications to both the N-terminus of the peptide sequence and the C-terminus of the peptide sequence with one or more non-peptide-based moieties.

In some embodiments, one or more terminal portions of a peptide can be connected to the parent portion peptide using a linking moiety. In some embodiments, the terminal portion can be separated from the parent peptide by 1, 2, 3, 4, 5, 6, 7, 8, 9, 10 or more residues. In some embodiments, the terminal portion can comprise any natural or unnatural amino acid, the N-methylated form of any natural or unnatural amino acid, and/or the D-stereoisomer of any natural or unnatural amino acid. In some embodiments, the terminal portion can be comprise norvaline, tert-butylglycine, phenylglycine, azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobutyric acid, (S)-2-amino-3-(1H-tetrazol-5-yl) propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy) acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine. (S)-2-amino-5-(3-methylguanidino) pentanoic acid, (S)-2-amino-3-(4-(aminomethyl)phenyl) propanoic acid, (S)-2-amino-3-(3-(aminomethyl)phenyl) propanoic acid, (S)-2-amino-4-(2-aminobenzo[d]oxazol-5-yl) butanoic acid, (S)-leucinol, (S)-valinol, (S)-tert-leucinol, (R)-3-methylbutan-2-amine, (S)-2-methyl-1-phenylpropan-1-amine, and(S)—N,2-dimethyl-1-(pyridin-2-yl) propan-1-amine, (S)-2-amino-3-(oxazol-2-yl) propanoic acid, (S)-2-amino-3-(oxazol-5-yl) propanoic acid, (S)-2-amino-3-(1,3,4-oxadiazol-2-yl) propanoic acid, (S)-2-amino-3-(1,2,4-oxadiazol-3-yl) propanoic acid, (S)-2-amino-3-(5-fluoro-1H-indazol-3-yl) propanoic acid, and/or(S)-2-amino-3-(1H-indazol-3-yl) propanoic acid.

In some embodiments, α-MSH analogs of the present disclosure can comprise modifications to the N-terminus of the peptide sequence with one or more of the following moieties: Ac-Nle; Ac-Arg; 7-Ahept; BzlSO₂, HyBA; HyPA; HymBA; MoPA, PyrPropHep; 2-Nac; Nba; Npa; Pba; Ppa; PyAA; or Tos.

In some embodiments, α-MSH analogs of the present disclosure can comprise modifications to the N-terminus which comprise a string of 5 or 6 Glu amino acids. In some embodiments, α-MSH analogs of the present disclosure can comprise modifications to the N-terminus which comprise a string of 5 or 6 Lys amino acids. In some embodiments, α-MSH analogs of the present disclosure can comprise modifications to the N-terminus which comprise a string of 5 or 6 amino acids, each independently selected from Glu or Lys. In some embodiments, α-MSH analogs of the present disclosure can comprise modifications to the N-terminus which comprise a string of 5 or 6 amino acids, each independently selected from Glu or Lys, comprising: L-L-L-L-L-L (SEQ ID NO: 105); G-L-L-L-L-L (SEQ ID NO: 106); L-G-L-L-L-L (SEQ ID NO: 107); L-L-G-L-L-L (SEQ ID NO: 108); L-L-L-G-L-L (SEQ ID NO: 109); L-L-L-L-G-L (SEQ ID NO: 110); L-L-L-L-L-G (SEQ ID NO: 111); G-G-L-L-L-L (SEQ ID NO: 112); G-L-G-L-L-L (SEQ ID NO: 113); G-L-L-G-L-L (SEQ ID NO: 114); G-L-L-L-G-L (SEQ ID NO: 115); G-L-L-L-L-G (SEQ ID NO: 116); L-G-G-L-L-L (SEQ ID NO: 117); L-G-L-G-L-L (SEQ ID NO: 118); L-G-L-L-G-L (SEQ ID NO: 119); L-G-L-L-L-G (SEQ ID NO: 120); L-L-G-G-L-L (SEQ ID NO: 121); L-L-G-L-G-L (SEQ ID NO: 122); L-L-G-L-L-G (SEQ ID NO: 123); L-L-L-G-G-L (SEQ ID NO: 124); L-L-L-G-L-G (SEQ ID NO: 125); L-L-L-L-G-G (SEQ ID NO: 126); G-G-G-L-L-L (SEQ ID NO: 127); G-G-L-G-L-L (SEQ ID NO: 128); G-G-L-L-G-L (SEQ ID NO: 129); G-G-L-L-L-G (SEQ ID NO: 130); G-L-G-G-L-L (SEQ ID NO: 131); G-L-G-L-G-L (SEQ ID NO: 132); G-L-G-L-L-G (SEQ ID NO: 133); G-L-L-G-G-L (SEQ ID NO: 134); G-L-L-G-L-G (SEQ ID NO: 135); G-L-L-L-G-G (SEQ ID NO: 136); L-L-L-G-G-G (SEQ ID NO: 137); L-L-G-L-G-G (SEQ ID NO: 138); L-L-G-G-L-G (SEQ ID NO: 139); L-L-G-G-G-L (SEQ ID NO: 140); L-G-L-L-G-G (SEQ ID NO: 141); L-G-L-G-L-G (SEQ ID NO: 142); L-G-L-G-G-L (SEQ ID NO: 143); L-G-G-L-L-G (SEQ ID NO: 144); L-G-G-L-G-L (SEQ ID NO: 145); L-G-G-G-L-L (SEQ ID NO: 146); L-L-G-G-G-G (SEQ ID NO: 147); L-G-L-G-G-G (SEQ ID NO: 148); L-G-G-L-G-G (SEQ ID NO: 149); L-G-G-G-L-G (SEQ ID NO: 150); L-G-G-G-G-L (SEQ ID NO: 151); G-L-L-G-G-G (SEQ ID NO: 152); G-L-G-L-G-G (SEQ ID NO: 153); G-L-G-G-L-G (SEQ ID NO: 154); G-L-G-G-G-L (SEQ ID NO: 155); G-G-L-L-G-G (SEQ ID NO: 156); G-G-L-G-L-G (SEQ ID NO: 157); G-G-L-G-G-L (SEQ ID NO: 158); G-G-G-L-L-G (SEQ ID NO: 159); G-G-G-L-G-L (SEQ ID NO: 160); G-G-G-G-L-L (SEQ ID NO: 161); L-G-G-G-G-G (SEQ ID NO: 162); G-L-G-G-G-G (SEQ ID NO: 163); G-G-L-G-G-G (SEQ ID NO: 164); G-G-G-L-G-G (SEQ ID NO: 165); G-G-G-G-L-G (SEQ ID NO: 166); G-G-G-G-G-L (SEQ ID NO: 167); or G-G-G-G-G-G (SEQ ID NO: 168).

In some embodiments, α-MSH analog compounds of the present disclosure comprise an N-terminal peptide consisting of a chain of about 15 to about 400 identical amino acids. In some embodiments, the N-terminal peptide comprises about 25 to about 300 identical amino acids, about 50 to about 200 identical amino acids, about 75 to about 150 identical amino acids, about 90 to about 120 identical amino acids, or about 100 or 110 identical amino acids. In some embodiments, the N-terminal peptide comprises: poly(glutamic acid) polypeptides (PGa), poly(aspartic acid) polypeptides (PAs), poly(lysine) polypeptides (PLy), poly(arginine) polypeptides (PAr), poly(histidine) polypeptides (PHi), poly(ornithine) polypeptides (POr), or combinations thereof (e.g., Ply-PGa-α-MSH).

In some embodiments, α-MSH analogs of the present disclosure can comprise modifications to the C-terminus of the peptide sequence with one or more of the following moieties: NH—CH₃; NH—CH₂—CH₃; NH—CH—(CH₃)₂, NH—CH₂—CH₂—CH₃, NH—CH₂—CH—(CH₃)₂, N(CH₃)₂, N(CH₂—CH₃)₂, OH, Trp-NH₂.

In some embodiments, α-MSH analogs of the present disclosure can comprise a peptide portion conjugated to a non-peptide-based portion. In some embodiments, α-MSH analogs of the present disclosure can comprise a peptide portion conjugated to a non-peptide portion-based selected from lipids, small molecules, RNA, DNA, polymers, or combinations thereof. In some embodiments, conjugates comprise covalent modifications introduced by reacting targeted amino acid residues or the termini of the peptide with an organic derivatizing agent that is capable of reacting with selected side-chains or terminal residues.

In some embodiments, the conjugation process may involve one or more of: PEGylation, lipidation, albumination, biotinylation, desthiobiotinylation, the addition of other peptide tails, or grafting into proteins.

In some embodiments, α-MSH analogs are conjugated to one or more anchors. In some embodiments, α-MSH analogs are conjugated to one or more anchors selected from: cholesterol oleate moiety, cholesteryl laurate moiety, an α-tocopherol moiety, a phytol moiety, an oleate moiety, an unsaturated cholesterol-ester moiety, or a lipophilic compound selected from acetanilides, anilides, aminoquinolines, benzhydryl compounds, benzodiazepines, benzofurans, cannabinoids, cyclic polypeptides, dibenzazepines, digitalis glycosides, ergot alkaloids, flavonoids, imidazoles, quinolines, macrolides, naphthalenes, opiates (such as, but not limited to, morphinans or other psychoactive drugs), oxazines, oxazoles, phenylalkylamines, piperidines, polycyclic aromatic hydrocarbons, pyrrolidines, pyrrolidinones, stilbenes, sulfonylureas, sulfones, triazoles, tropanes, and vinca alkaloids.

In some embodiments, α-MSH analogs are conjugated to a hydrophilic polymer. In some embodiments, the conjugate includes a hydrophilic polymer selected from: polyalkylene oxide homopolymers, polypropylene glycols, polyoxyethylenated polyols and copolymers thereof. In some embodiments, the conjugate includes polyethylene glycol (PEG). In some embodiments, α-MSH analogs are conjugated to an albumin binding polypeptide. In some embodiments, α-MSH analogs are conjugated to cell penetrating polypeptides.

In some embodiments, α-MSH analogs are conjugated to lipidic moiety. In some embodiments, α-MSH analogs are conjugated to a lipidic moiety selected from: fatty acids, phospholipids, and sterols. In some embodiments, α-MSH analogs are directly conjugated to the lipidic moiety. In some embodiments, the lipidic moiety is conjugated to a polypeptide-PEG conjugate. In some embodiments, α-MSH analogs are conjugated to a lipidic moiety selected from Palm-PEG8-G-G-Ser-Tyr (SEQ ID NO: 169), Ac—K(Palm)-G-G-Ser-Tyr (SEQ ID NO: 170), capric acid (C10), lauric acid (C12), myristic acid (C14), palmitic acid (C16), stearic acid (C18), PEG4, or PEG8.

In some embodiments, two or more α-MSH analogs dimerized by conjugation to the same lipidic moiety.

### Cyclic Peptides

In some embodiments, α-MSH analogs of the present disclosure can comprise cyclic peptides having one or more bridging moieties (e.g., cyclic structure, staple, bridge, etc.), as compared to natural α-MSH (or fragments thereof, e.g., as in Table 1). Peptide stapling/bridging is a macrocyclization approach in which peptides are covalently modified through the formation of a chemical linkage (e.g., staple, bridge moiety, etc.) between the side chains of two amino acids. More specifically, peptides are rendered macrocyclic by formation of covalent bonds between atoms present within the linear peptide and atoms of a bridging moiety. Stapling/bridging can be used to constrain peptides into preferred bioactive conformations (reducing conformational flexibility and degrees of rotational freedom), thereby improving affinity for specific receptor targets and improving overall pharmacokinetics. Without being bound by theory, the residues being linked are generally located on the same face of the peptide helix and separated by one, two, or three helical turns (e.g., a first amino acid at position (2) is linked to a second amino acid at position z+4, z+7, or z+11). In some embodiments, bridging moieties may comprise one or more chemical bonds between two adjacent or non-adjacent amino acids, unnatural amino acids, non-amino acid residues or combinations thereof. In some embodiments, such chemical bonds may be between one or more functional groups on adjacent or non-adjacent amino acids, unnatural amino acids, non-amino acid residues or combinations thereof.

Examples of bridging moieties/peptide staples for use with compounds of the present disclosure include, but are not limited to: Amide-based (e.g., lactam) bridges; aromatic-ring-based bridges; hydrocarbon chains; Alkene-based hydrocarbon bridges (e.g., using Fmoc-S-2-(2′-pentenyl)alanine); Triazole-based Click bridges, such as copper(I)-catalyzed Huisgen 1,3-dipolar cycloaddition reactions between side chain azido and alkynyl moieties (e.g., Fmoc-L-Nle(εN₃) and Fmoc-D-Pra) (see S. Kawamoto, et al., J. Med. Chem. 2012, 55 (3), 1137-1146); dialkenyl staples (e.g., 1,4-Diethynylbenzene, diethynylpentane, diethynylamines) for stapling linear diazido-peptides; Sulfide-bonded disulfide, thioether and bis-thioether bridges; Perfluorobenzene bridges; or combinations thereof.

In some embodiments, bridging moieties comprise an amide bond between an amine functionality and a carboxylate functionality, each present in an amino acid, unnatural amino acid or non-amino acid residue side chain. In some embodiments, the amine or carboxylate functionalities are part of a non-amino acid residue or unnatural amino acid residue. In some embodiments, the bridging moiety comprises an amide bond produced by the reaction of the side chains of the following pairs of amino acids: lysine and glutamate; lysine and aspartate; ornithine and glutamate; ornithine and aspartate; homolysine and glutamic acid; homolysine and aspartic acid; and other combinations of amino acids, unnatural amino acids or non-amino acid residues comprising a primary amine and a carboxylic acid.

In some embodiments, bridging moieties may comprise bonds formed between residues that may include, but are not limited to(S)-2-amino-5-azidopentanoic acid; (S)-2-aminohept-6-enoic acid; (S)-2-aminopent-4-ynoic acid; (S)-2-aminopent-4-enoic acid; or combinations thereof.

In some embodiments, bridging moieties are formed through cyclization reactions using olefin metathesis.

In some embodiments, the bridging moiety comprises a disulfide bond formed between two thiol containing residues. In some embodiments, the bridging moiety comprises one or more thioether bonds. Such thioether bonds may include those found in cyclo-thioalkyl compounds. These bonds can be formed during a chemical cyclization reaction between chloro acetic acid N-terminal modified groups and cysteine residues. In some embodiments, bridging moieties comprise one or more triazole ring.

In some embodiments, bridging moieties comprise one or more hydrocarbon chains (linear or branched), and/or hydrocarbon rings (cyclic, heterocyclic, aromatic, heteroaromatic). In some embodiments, hydrocarbon bridging moieties may be introduced by reaction with reagents containing multiple reactive halides, including, but not limited to poly(bromomethyl)benzenes, poly(bromomethyl)pyridines, poly(bromomethyl) alkyl benzenes and/or (E)-1,4-dibromobut-2-ene. Examples of Poly(bromomethyl)benzene molecules of the present disclosure can include 1,2-bis(bromomethyl)benzene; 1,3-bis(bromomethyl)benzene; and 1,4-bis(bromomethyl)benzene.

In some embodiments, the thiol group of a cysteine residue is cross-linked with another cysteine residue to form a disulfide bond. In some embodiments, thiol groups of cysteine residues react with bromomethyl groups of poly(bromomethyl)benzene molecules to form stable linkages (see, e.g., Timmerman et al., ChemBioChem (2005) 6:821-824, the contents of which are incorporated herein by reference in their entirety).

In some embodiments, Bis-, tris- and tetrakis(bromomethyl)benzene molecules can be used to generate bridging moieties to produce peptides with one, two or three loops, respectively. Bromomethyl groups of a poly(bromomethyl)benzene molecule may be arranged on the benzene ring on adjacent ring carbons (ortho- or o-), with a ring carbon separating the two groups (meta- or m-) or on opposite ring carbons (para- or p-). In some embodiments, m-bis(bromomethyl)benzene (i.e., m-dibromoxylene), o-bis(bromomethyl)benzene (i.e., o-dibromoxylene) and/or p-bis(bromomethyl)benzene (i.e., p-dibromoxylene) are used to form cyclic peptides. In some embodiments, thiol groups of cysteine residues react with other reagents comprising one or more bromo functional groups to form stable linkages. Such reagents may include, but are not limited to poly(bromomethyl)pyridines (e.g., 2,6-bis(bromomethyl)pyridine), poly(bromomethyl) alkyl benzenes (e.g., 1,2-bis(bromomethyl)-4-alkylbenzene) and/or (E)-1,4-dibromobut-2-ene.

In some embodiments, a side chain amino group and a terminal amino group are cross-linked with disuccinimidyl glutarate (see, e.g., Millward et al., J. Am. Chem. Soc. (2005) 127:14142-14143. In some embodiments, an enzymatic method is used which relies on the reaction between (1) a cysteine and (2) a dehydroalanine or dehydrobutyrine group, catalyzed by a lantibiotic synthetase, to create the thioether bond (see, e.g., Levengood et al., Bioorg. and Med. Chem. Lett. (2008) 18:3025-3028). The dehydro functional group can also be generated chemically by the oxidation of selenium containing amino acid side chains incorporated during translation (see, e.g., Seebeck et al., J. Am. Chem. Soc. 2006).

In some embodiments, bridging moieties comprise an aromatic, 6-membered ring (e.g., benzene). In some embodiments, bridging moieties comprise a heterocyclic, 6-membered ring which includes one nitrogen atoms (e.g., pyridine). In some embodiments, bridging moieties comprise a heterocyclic, 6-membered ring which includes two nitrogen atoms (e.g., pyridazine, pyrimidine, pyrazine). In some embodiments, bridging moieties comprise a heterocyclic, 6-membered ring which includes three nitrogen atoms (e.g., triazanes). In some embodiments, bridging moieties comprise a heterocyclic, 5-membered ring which includes one nitrogen atoms (e.g., pyrrole). In some embodiments, bridging moieties comprise a heterocyclic, 5-membered ring which includes two nitrogen atoms (e.g., imidazole, pyrazole). In some embodiments, bridging moieties comprise a heterocyclic, 5-membered ring which includes three nitrogen atoms (e.g., triazoles).

In some embodiments, α-MSH analogs of the present disclosure can comprise a bridging link at the AA3 locus (i.e., Ser³ location). In some embodiments, α-MSH analogs of the present disclosure can comprise a bridging link at the AA4 locus (i.e., Met⁴ location). In some embodiments, α-MSH analogs of the present disclosure can comprise a bridging link at the AA5 locus (i.e., Glu⁵ location). In some embodiments, α-MSH analogs of the present disclosure can comprise a bridging link at the AA9 locus (i.e., Trp⁹ location). In some embodiments, α-MSH analogs of the present disclosure can comprise a bridging link at the AA10 locus (i.e., Gly¹⁰ location). In some embodiments, α-MSH analogs of the present disclosure can comprise a bridging link at the AA11 locus (i.e., Lys¹¹ location). In some embodiments, α-MSH analogs of the present disclosure can comprise a bridging link at the AA12 locus (i.e., Pro¹² location).

In some embodiments, α-MSH analogs of the present disclosure can comprise a bridging link at the AA3 locus (i.e., Ser³ location) and at the AA12 locus (i.e., Pro¹² location). In some embodiments, α-MSH analogs of the present disclosure can comprise a bridging link at the AA4 locus (i.e., Met⁴ location) and at the AA10 locus (i.e., Gly¹⁰ location). In some embodiments, α-MSH analogs of the present disclosure can comprise a bridging link at the AA4 locus (i.e., Met⁴ location) and at the AA11 locus (i.e., Lys¹¹ location). In some embodiments, α-MSH analogs of the present disclosure can comprise a bridging link combination selected from: [Lys⁴, Pra¹¹], [Cys⁴, Cys¹⁰], [Cys⁴, Cys¹¹], [Maa⁴, Cys¹⁰], [Mpa⁴, Cys¹⁰], [Nle⁴, Gly¹⁰], or [Hcy⁴, Cys^{10].}

In some embodiments, α-MSH analogs of the present disclosure can comprise a bridging link at the AA5 locus (i.e., Glu⁵ location) and at the AA9 locus (i.e., Trp⁹ location). In some embodiments, α-MSH analogs of the present disclosure can comprise a bridging link at the AA5 locus (i.e., Glu⁵ location) and at the AA10 locus (i.e., Gly¹⁰ location). In some embodiments, α-MSH analogs of the present disclosure can comprise a bridging link at the AA5 locus (i.e., Glu⁵ location) and at the AA11 locus (i.e., Lys¹¹ location). In some embodiments, AA5 may be either a L- or D-amino acid having an omega-amino or carboxyl group in the side chain, for example AA5 may be α,γ-diaminopropionic acid, α,γ-diaminobutyric acid, Orn, Lys, α-aminoadipic acid, α-aminopimelic acid, or higher (i.e., alkyldionic acids containing more than 7 carbons) homologs, Glu or Asp. In some embodiments, AA5 may be Lys(N3), DLys(N3), Ndab, NGlu, Cys, α-MeCys, Pra or Orn(N3). In some embodiments, AA9 may be Dab, NMeTrp, Trp(Me), Orn, Ndab, Cys or α-MeCys, In some embodiments, AA10 may be either a L- or D-amino acid having an omega-amino or carboxyl group in the side chain, for example, AA10 may be diaminopropionic acid, α,γ-diaminobutyric acid, Orn, Lys, α,β-aminoadipic acid, α-aminopimelic acid, or higher homologs, Glu or Asp. In some embodiments, AA10 may be Trp, Pra, Hpra, Cys, Ala, Dab, Dab(N3), Lys(N3) or D-Lys(N3). In some embodiments, AA11 may be a L- or D-amino acid having an omega-amino or carboxyl group in the side chain, for example, AA11 may be α,β-diaminopropionic acid, α,γ-diaminobutyric acid, Orn, Lys, α-aminoadipic acid, α-aminopimelic acid, or higher homologs, Glu or Asp. In some embodiments, AA11 may be Pra, Cys, Gly, Asn, Hgln, or Lys(Me) 2. In some embodiments, α-MSH analogs of the present disclosure can comprise a bridging link combination selected from: [Glu⁵, Dab⁹], [Lys⁵, Pra¹⁰], [NGlu⁵, Orn⁹], [Glu⁵, NDab⁹], [NGlu⁵, NDab⁹], [NDab⁵, NDab⁹], [Cys⁵, Cys⁹], [α-Me-Cys⁵, α-Me-Cys⁹], [Dab⁵, Dab¹⁰], [Orn⁵, Pra¹⁰], [Lys⁵, HPra¹⁰], [Pra⁵, Lys¹⁰], [Orn⁵, HPra¹⁰], [D-Lys⁵, Pra¹⁰], [Lys⁵, D-Pra¹⁰], [Asp⁵. Lys¹⁰], [Glu⁵, Lys¹⁰], [Cys⁵, Cys¹⁰], [Cys⁵, Cys¹¹], [Glu⁵, Orn¹⁰], [Lys(N3) 5. Pra¹⁰], [Asp⁵, Lys¹¹], [Glu⁵, Lys¹¹], [Lys⁵. Asp¹¹], or [Suc⁵, Lys¹⁰].

In some embodiments, α-MSH analogs of the present disclosure can comprise a bridging link at the AA6 locus (i.e., His⁶ location) and at the AA9 locus (i.e., Trp⁹ location). In some embodiments, α-MSH analogs of the present disclosure can comprise a bridging link at the AA6 locus (i.e., His⁶ location) and at the AA10 locus (i.e., Gly¹⁰ location). In some embodiments, α-MSH analogs of the present disclosure can comprise a bridging link at the AA6 locus (i.e., His⁶ location) and at the AA11 locus (i.e., Lys¹¹ location). In some embodiments, α-MSH analogs of the present disclosure can comprise a bridging link combination selected from: [Tyr⁶, Trp⁹], [Tyr⁶, Gly¹⁰], [Tyr⁶, Lys¹¹].

In some embodiments, α-MSH analogs of the present disclosure can comprise one or more Lactam-bridges to provide cyclic α-MSH peptide analogs. Examples of Lactam-bridges for use in α-MSH peptide analogs include those presented in U.S. Pat. No. 9,273,098, the contents of which are incorporated herein by reference in their entirety as related to lactam-bridges for use in MCR-targeting compound such as α-MSH peptide analogs.

In some embodiments, α-MSH analogs of the present disclosure can comprise one or more amide-based lactam bridges, carbonyl-based lactam bridges, or combinations thereof. In some embodiments, α-MSH analogs of the present disclosure can comprise a bridging moiety comprising one or more bridging groups selected from: (CH₂)₁₋₆—C(═O)—NH—(CH₂)₁₋₆; (CH₂)₁₋₆—NH—C(═O)—(CH₂)₁₋₆; (CH₂)₁₋₆—C(═O)—(CH₂)₁₋₆; C(═O)—(CH₂)₁₋₆—; (CH₂)₁₋₆—C(═O)—NH—C(═O)—(CH₂)₁₋₆—; (CH₂)₁₋₆—NH—C(═O)—(CH₂)₁₋₆—NH—C(═O)—(CH₂)₁₋₆; (CH₂)₁₋₆—NH—C(═O)—NH—(CH₂)₁₋₆; [Nitrobenzene]—NH—(CH₂)₁₋₆—NH—C(═O)—(CH₂)₁₋₆; [Phenylamine]—NH—(CH₂)₁₋₆—NH—C(═O)—(CH₂)₁₋₆; [Nitrobenzene]—NH—(CH₂)₁₋₆—N(CH₃)—C(═O)—(CH₂)₁₋₆; or [Phenylamine]—NH—(CH₂)₁₋₆—N(CH₃)—C(═O)—(CH₂)₁₋₆.

### Amino Acid Variants and Derivatization

Amino acids, as used herein, include both natural amino acids (i.e., the 20 proteinogenic amino acids) and unnatural amino acids. The term also includes amino acids bearing a conventional amino protecting group (e.g., acetyl or benzyloxycarbonyl), as well as natural and unnatural amino acids protected at the carboxy terminus (e.g., as a (C1-C6)alkyl, phenyl or benzyl ester or amide, or as an alpha-methylbenzyl amide). Other suitable amino and carboxy protecting groups are known to those skilled in the art (see, e.g., Greene, et al., Protecting Groups In Organic Synthesis; second edition, 1991, New York, John Wiley & sons, Inc.). Peptides and/or peptide compositions of the present invention may also include modified amino acids.

Examples of unnatural amino acids useful for the modifying peptides of the present disclosure include, but are not limited to: 1,2,3,4-tetrahydroisoquinoline-1-carboxylic acid, 1-amino-2,3-hydro-1H-indene-1-carboxylic acid, homolysine, homoarginine, homoserine, 2-aminoadipic acid, 3-aminoadipic acid, beta-alanine, aminopropionic acid, 2-aminobutyric acid, 4-aminobutyric acid, 5-aminopentanoic acid, 5-aminohexanoic acid, 6-aminocaproic acid, 2-aminoheptanoic acid, 2-aminoisobutyric acid, 3-aminoisobutyric acid, 2-aminopimelic acid, desmosine, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylpentylglycine, naphthylalanine, ornithine, pentylglycine, thioproline, norvaline, tert-butylglycine, phenylglycine, azatryptophan, 5-azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, (S)-2-amino-3-(1H-tetrazol-5-yl) propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy) acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine, (S)-2-amino-5-azidopentanoic acid, (S)-2-aminohept-6-enoic acid, (S)-2-aminopent-4-ynoic acid, (S)-2-aminopent-4-enoic acid, (S)-2-amino-5-(3-methylguanidino) pentanoic acid, (S)-2-amino-3-(4-(aminomethyl)phenyl) propanoic acid, (S)-2-amino-3-(3-(aminomethyl)phenyl) propanoic acid, (S)-2-amino-4-(2-aminobenzo[d]oxazol-5-yl) butanoic acid, (S)-leucinol, (S)-valinol, (S)-tert-leucinol, (R)-3-methylbutan-2-amine, (S)-2-methyl-1-phenylpropan-1-amine, and(S)—N,2-dimethyl-1-(pyridin-2-yl) propan-1-amine, (S)-2-amino-3-(oxazol-2-yl) propanoic acid, (S)-2-amino-3-(oxazol-5-yl) propanoic acid, (S)-2-amino-3-(1,3,4-oxadiazol-2-yl) propanoic acid, (S)-2-amino-3-(1,2,4-oxadiazol-3-yl) propanoic acid, (S)-2-amino-3-(5-fluoro-1H-indazol-3-yl) propanoic acid, and(S)-2-amino-3-(1H-indazol-3-yl) propanoic acid, (S)-2-amino-3-(oxazol-2-yl) butanoic acid, (S)-2-amino-3-(oxazol-5-yl) butanoic acid, (S)-2-amino-3-(1,3,4-oxadiazol-2-yl) butanoic acid, (S)-2-amino-3-(1,2,4-oxadiazol-3-yl) butanoic acid, (S)-2-amino-3-(5-fluoro-1H-indazol-3-yl) butanoic acid, and(S)-2-amino-3-(1H-indazol-3-yl) butanoic acid, 2-(2′MeOphenyl)-2-amino acetic acid, tetrahydro 3-isoquinolinecarboxylic acid, and stereoisomers thereof (including, but not limited, to D and L isomers).

Additional unnatural amino acids useful for the modifying peptides of the present disclosure include fluorinated amino acids (i.e., amino acids with one or more carbon bound hydrogen atoms being replaced by fluorine). Examples of fluorinated amino acids include, but are not limited to: 3-fluoroproline, 3,3-difluoropyrroline, 4-fluoroproline, 4,4-difluoropyrroline, 3,4-difluoropyrroline, 3,3,4,4-tetrafluoropyrroline, 4-fluorotryptophan, 5-fluorotryptophan, 6-fluorotryptophan, 7-fluorotryptophan, and stereoisomers thereof.

Additional unnatural amino acids useful for the modifying peptides of the present disclosure include amino acids that are disubstituted at the α-carbon. These include amino acids in which the two substituents on the α-carbon are the same, for example α-amino isobutyric acid, and 2-amino-2-ethyl butanoic acid. These also include amino acids where the substituents are different, for example α-methylphenylglycine and α-methylproline. Further the substituents on the α-carbon may be taken together to form a ring, for example 1-aminocyclopentanecarboxylic acid, 1-aminocyclobutanecarboxylic acid, 1-aminocyclohexanecarboxylic acid, 3-aminotetrahydrofuran-3-carboxylic acid, 3-aminotetrahydropyran-3-carboxylic acid, 4-aminotetrahydropyran-4-carboxylic acid, 3-aminopyrrolidine-3-carboxylic acid, 3-aminopiperidine-3-carboxylic acid, 4-aminopiperidine-4-carboxylic acid, and stereoisomers thereof.

Additional unnatural amino acids useful for the modifying peptides of the present disclosure include analogs of tryptophan in which the indole ring system is replaced by another 9 or 10 membered bicyclic ring system comprising 0, 1, 2, 3 or 4 heteroatoms independently selected from N, O, or S. Each ring system may be saturated, partially unsaturated, or fully unsaturated. The ring system may be substituted by 0, 1, 2, 3, or 4 substituents at any substitutable atom. Each substituent is independently selected from H, F, Cl, Br, CN, COOR, CONRR′, oxo, OR, and NRR′. Each R and R′ is independently selected from H, C₁-C₂₀ alkyl, C₁-C₂₀ alkyl-O—C₁-C₂₀ alkyl.

In some embodiments, tryptophan analogs useful for the modifying peptides of the present disclosure include 5-fluorotryptophan [(5-F)W], 5-methyl-O-tryptophan [(5-MeO)W], 1-methyltryptophan [(1-Me-W) or (1-Me)W], D-tryptophan (D-Trp), azatryptophan (including, but not limited to, 4-azatryptophan, 7-azatryptophan and 5-azatryptophan), 5-chlorotryptophan, 4-fluorotryptophan, 6-fluorotryptophan, 7-fluorotryptophan, and stereoisomers thereof. Except where indicated to the contrary, the term “azatryptophan” and its abbreviation, “azaTrp,” as used herein, refer to 7-azatryptophan.

Modified amino acid residues useful for the modifying peptides of the present disclosure include amino acids which are: chemically blocked (reversibly or irreversibly); chemically modified on their N-terminal amino group; chemically modified on their side chain groups; or chemically modified in the amide backbone (e.g., N-methylated, D or L stereoisomers). Examples of modified amino acids include: methionine sulfoxide; methionine sulfone; aspartic acid-(beta-methyl ester); N-ethylglycine; and alanine carboxamide.

In some embodiments, examples of unnatural amino acids useful for the modifying peptides of the present disclosure include those listed in Table 2 of US 2011/0172126, the contents of which are incorporated herein by reference in their entirety as related to unnatural amino acids for the modifying peptides.

In some embodiments, amino acids for use in the present disclosure are modified using an organic proteinaceous or non-proteinaceous derivatizing agent. In some embodiments, amino acids for use in the present disclosure are modified using post-translational modification. In some embodiments, modifications are introduced by reacting targeted amino acid residues of the peptide with an organic derivatizing agent that is capable of reacting with selected side-chains or terminal residues. In some embodiments, modifications are introduced by harnessing mechanisms of post-translational modifications that function in selected recombinant host cells. Certain post-translational modifications are the result of the action of recombinant host cells on an expressed peptide. As one example, glutaminyl and asparaginyl residues are frequently post-translationally deamidated to the corresponding glutamyl and aspartyl residues under certain post-translational conditions (e.g., under mildly acidic conditions). Other post-translational modifications include: hydroxylation of proline and lysine; phosphorylation of hydroxyl groups of tyrosinyl, seryl or threonyl residues; and methylation of the alpha-amino groups of lysine, arginine, and histidine side chains (see, e.g. Creighton et al., Proteins: Structure and Molecular Properties, W.H. Freeman & Co., San Francisco, 1983, pp. 79-86).

In some embodiments, amino acid modifications include the bonding of non-proteinaceous polymers to peptides of the present disclosure. Examples of non-proteinaceous polymers include hydrophilic synthetic polymers (i.e., non-natural polymers), such as hydrophilic polyvinyl polymers (e.g., polyvinylalcohol and polyvinylpyrrolidone). The Examples of non-proteinaceous polymers also include polyethylene glycol, polypropylene glycol and polyoxyalkylenes. In some embodiments, amino acid modifications include the bonding of non-proteinaceous polymers to peptides of the present disclosure, as described in U.S. Pat. Nos. 4,640,835, 4,496,689, 4,301,144, 4,670,417, 4,791,192, and 4,179,337; the contents of which are each incorporated herein by reference in their entirety, as related to amino acid modifications for use in the present disclosure.

According to the present disclosure, the following abbreviations have the meanings as given.

TABLE 2
Structure abbreviations

| Abbreviation | Full Name/Structure |
|---|---|
| Abu | gamma-amino butyric acid |
| 2-Abz | 2-amino benzoic acid |
| 3-Abz | 3-amino benzoic acid |
| 4-Abz | 4-amino benzoic acid |
| Ac-Glu | Acetyl glutamic acid |
| Achc | 1-amino-cyclohexane-1-carboxylic acid |
| Ac-Lys(N3) | Acetyl azidolysine |
| Ac-Met | Acetyl methionine |
| Ac-Nle | Acetyl norleucine |
| Acpc | 1-amino-cyclopropane-1-carboxylic acid |
| Ac-R5 | Acetyl R-2-(4-pentenyl)alanine |
| Ac-R8 | Acetyl R-2-(7-octenyl) alanine |
| Ac-S5 | Acetyl S-2-(4-pentenyl) alanine |
| Ac-Ser | Acetyl serine |
| Ac-Tyr | Acetyl tyrosine |
| 12-Ado | 12-amino-dodecanoic acid |
| Agp | 2-amino-3-guanidino-propionic acid |
| 7-Ahept | 7-amino-heptanoic acid |
| Aib | alpha-aminoisobutyric acid |
| Aic | 2-aminoindane-2-carboxylic acid |
| 6-Ahx | 6-amino-hexanoic acid |
| alphaMeCys | Alpha methyl cysteine |
| Amb | 4-(aminomethyl)-benzoic acid |
| Amc | 4-(aminomethyl)-cyclohexane-carboxylic acid |
| 8-Aoc | 8-amino-octanoic acid |
| Arg(Tos) | para-tosyl-arginine |
| Arg(Me) | Methyl-arginine |
| ArgMe2 | N_{ω},N′_{ω}-dimethyl-L-arginine |
| Asn-NH2 | Asparagine amide |
| Asp(anilino) | beta-anilino-aspartic acid |
| Asp(3-Cl-anilino) | beta-(3-chloro-anilino)-aspartic acid |
| Asp(3,5-diCl-anilino) | beta-(3,5-dichloro anilino)-aspartic acid |
| Atc | 2-aminotetralin-2-carboxylic acid |
| 11-Aun | 11-amino-undecanoic acid |
| AVA | 5-amino-valeric acid |
| BzlSO₂ | benzosulfonyl group |
| Bip | biphenyl-alanine |
| BrAc-Glu | Bromoacetyl glutamic acid |
| BrAc-Nle | Bromoacetyl norleucine |
| Can | cyclic analog |
| Cba | cyclobutyl-alanine |
| Cha | cyclohexyl-alanine |
| Chg | cyclohexyl-glycine |
| Cmpi | 4-caboxymethyl-piperazine |
| Cpna | cyclopentyl-alanine |
| Cpra | cyclopropyl-alanine |
| Cys-NH2 | cysteinamide |
| Daa | dibasic amino acid |
| Dab | 2,4-diamino-butyric acid |
| Dab(Ac) | 2-amino-4-acetylamino-butyric acid |
| Dab(N3) | (S)-2-Amino-4-azidobutanoic acid |
| Dap | 2,4-diamino-propionic acid |
| Dip | 3,3-diphenyl-alanine |
| Disc | 1,3-dihydro-2H-isoindole-carboxylic acid |
| DLys(N3) | (R)-2-amino-6-azidohexanoic acid |
| D-NMePhe | N-methyl-D-phenylalanine |
| DPhe | D-phenylalanine |
| Dphe(4-CF3) | 4-trifluoromethyl-phenylalanine |
| Dphe(4-F) | 4-fluoro-D-phenylalanine |
| DPra | D-propargylglycine |
| Dpr(beta-Ala) | (3-aminopropionyl)-alpha,beta-diamino- |
| | propionic acid |
| DTyrMe | O-methyl-D-tyrosine |
| GAA | epsilon-guanidino-acetic acid |
| GBzA | 4-guanidino-benzoic acid |
| B-Gpa | 3-guanidino-propionic acid |
| Gly-NH2 | Glycineamide |
| GVA(Cl) | beta-chloro-epsilon-guanidino valeric acid |
| HArg | Homoarginine |
| Hcha | homocyclohexy-lalanine |
| Hcy | homo-cysteine |
| Hep | heptanoyl |
| Hgln-NH2 | Homoglutaminamide |
| hGlu | Homoglutamic acid |
| His(1-Me) | 1-methyl-histidine |
| His(3-Me) | 3-methyl-histidine |
| Hle | homo-leucine |
| Hphe | homo-phenylalanine |
| HPra | Homopropargylglycine |
| HyBA | 3,5-hydroxybenzoic acid |
| HymBA | 3-hydroxy-5-methoxybenzeneacetic acid |
| Hyp | hydroxy-proline |
| HyPA | 3,5-dihydroxyphenylacetic acid |
| Hyp(Bzl) | benzyl-hydroxy-proline |
| Igl | indanyl-glycine |
| Ile | Isoleucine |
| Inp | isonipecotic acid |
| Lys(N3) | (s)-2-amino-6-azidohexanoic acid |
| LysMe2-NH2 | N_{ω}-dimethyl-lysine amide |
| Lys-NH2 | Lysine amide |
| Lys(Z) | N-epsilon-benzyloxycarbonyl-lysine |
| Maa | 2-Mercapto-acetic acid |
| MoPA | (3-methoxyphenyl)acetic acid |
| Mpa | 3-Mercapto-propicnic acid |
| 2-Nac | 2-naphthyl-acetic acid |
| 1-Nal | 3-(1-naphthyl)-alanine |
| 2-Nal | 3-(2-naphthyl)-alanine |
| 2-Nal(N-Bzl) | N-benzyl-3-(2-naphthyl)-alanine |
| 2-Nal(N-PhEt) | N(2-phenylethyl)-3-(2-naphthyl)-alanine |
| Nba | 4-(naphthalen-2-yl)-butanoic acid |
| NDab | N-(2-aminoethyl)glycine |
| NGlu | N-(2-carboxyethyl)glycine |
| Nle | norleucine |
| NMeArg | N-methyl arginine |
| NMeDPhe | N-methyl D-phenylalanine |
| NMeHis | N-methyl histidine |
| NMe-Phe | N-methyl phenylalanine |
| NMeTrp | N-methyl tryptophan |
| Npa | 3-(naphthalen-1-yl)-propanoic acid |
| Nva | norvaline |
| (Nlys)Gly | N-(4-aminobutyl)-glycine |
| Octanoyl-PEG8-G | Octanoyl polyethylene glycol-8 glycine |
| Octanoyl-Ser | Octanoyl serine |
| Orn | ornithine |
| Orn(N3) | (S)-2-amino-5-azidopentanoic acid |
| Pal | Pyridyl-alanine |
| Pba | 4-phenyl-butanoic acid |
| Pen | penicillamine |
| Ppa | 3-phenyl-propionic acid |
| Pra | propargyl-glycine |
| Phg | phenyl-glycine |
| Phe(2-Me) | 2-methyl-phenylalanine |
| Phe(2-CN) | 2-cyano-phenylalanine |
| Phe(2-Cl) | 2-chloro-phenylalanine |
| Phe(2-F) | 2-fluoro-phenylalanine |
| Phe(2,4-Cl) | 2,4-dichloro-phenylalanine |
| D-Phe (2,4-DiCl) | 2,4-dichloro-D-phenylalanine |
| Phe(2,4-Me) | 2,4-dimethyl-phenylalanine |
| Phe(3-Me) | 3-methyl-phenylalanine |
| Phe(3-CN) | 3-cyano-phenylalanine |
| Phe(3-Cl) | 3-chloro-phenylalanine |
| Phe(3-F) | 3-fluoro-phenylalanine |
| Phe(3-Br) | 3-bromo-phenylalanine |
| Phe(3,4-diCl) | 3,4-dichloro-phenylalanine |
| Phe(3,4-diF) | 3,4-difluoro-phenylalanine |
| Phe(3,4-diOMe) | 2,4-dimethoxy-phenylalanine |
| Phe(4-Me) | 4-methyl-phenylalanine |
| Phe(4-CN) | 4-cyano-phenylalanine |
| Phe(4-Cl) | 4-chloro-phenylalanine |
| Phe(4-F) | 4-fluoro-phenylalanine |
| Phe(4-Br) | 4-bromo-phenylalanine |
| Phe(4-Me) | 4-methyl-phenylalanine |
| Phe(4-NH2) | 4-amino-Phenylalanine |
| Phe-NH2 | Phenylalanine amide |
| PheOCH3 | O-methyl-tyrosine |
| Phe(4-Ph) | 4-phenyl-phenylalanine |
| Phe(4-CF₃) | 4-trifluoromethyl-phenylalanine |
| Pip | pipecolic acid |
| Pra | Propargylglycine |
| Pra-NH2 | Propargyl-glycineamide |
| Pro-NH2 | Proline amide |
| 3-Pya | 3-pyridyl-alanine |
| PyAA | 4-pyridylacetyl acid |
| PyrProp | pyridinepropionyl group; |
| Qal(2′) | beta-(2-quinolyl)-alanine |
| R5 | R-2-(4-pentenyl)alanine |
| R8 | R-2-(7-octenyl) alanine |
| S5 | S-2-(4-pentenyl) alanine |
| S5-NH2 | S-2-(4-pentenyl) alanine amide |
| S8-NH2 | S-2-(7-octenyl) alanine amide |
| Sal | 3-styryl-alanine |
| Sar | sarcosine |
| Ser(Bzl) | O-benzyl-serine |
| Succ | succinyl |
| Thr(OBzl) | O-benzyl-threonine |
| Tic | 1,2,3,4-tetrahydroisoquinoline-3-carboxylic acid |
| Tiq | 1,2,3,4-tetrahydroisoquinoline-1-carboxytic acid |
| Thi | thienylalanine |
| Tle | tert-butylalanine |
| Tos | tosyl group |
| Tpi | 1,2,3,4-tetrahydronorharman-3-carboxylic acid |
| Trp(4-Cl) | 4-chloro-tryptophan |
| Trp(4-F) | 4-fluoro-tryptophan |
| Trp(5-Cl) | 5-chloro-tryptophan |
| Trp(5Cp) | 5-cyclopropyl-tryptophan |
| Trp(5-F) | 5-fluoro-tryptophan |
| Trp(5-Me) | 5-methyl-tryptophan |
| Trp(5-CF3) | 5-trifluoromethyl-tryptophan |
| Trp(6-Cl) | 6-chloro-tryptophan |
| Trp(6-F) | 6-fluoro-tryptophan |
| Trp(6-Me) | 6-methyl-tryptophan |
| Trp(7-Me) | 7-methyl-tryptophan |
| TrpMe | 1-methyl-tryptophan |
| Trp-NH2 | Tryptophan amide |
| Tyr(Me) | O-methyl-tyrosine |
| Tyr(Bzl) | O-benzyl-tyrosine |
| Tyr(BzlDiCl 2,6) | O-(2,6 dichloro)benzyl-tyrosine |
| Val-NH2 | Valine amide |

### Exemplary Embodiments of α-MSH Analogs

In some embodiments, α-MSH analogs of the present disclosure comprise a peptide sequence of the following Formula (I): Z-XAA1-XAA2-XAA3-XAA4-XAA5-XAA6-XAA7-XAA8-XAA9-XAA10-XAA11-XAA12-XAA13-Y; wherein

Z is absent, or comprises an N-terminus sequence selected from: Ac, PBA, octanoyl-PEG-GG, PyAA, HyBA, HyPA, MoPA, HymBA, C₁-C₁₀, norvaline, tert-butylglycine, phenylglycine, azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, aminoisobutyric acid, (S)-2-amino-3-(1H-tetrazol-5-yl) propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy) acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine. (S)-2-amino-5-(3-methylguanidino) pentanoic acid, (S)-2-amino-3-(4-(aminomethyl)phenyl) propanoic acid, (S)-2-amino-3-(3-(aminomethyl)phenyl) propanoic acid, (S)-2-amino-4-(2-aminobenzo[d]oxazol-5-yl) butanoic acid, (S)-leucinol, (S)-valinol, (S)-tert-leucinol, (R)-3-methylbutan-2-amine, (S)-2-methyl-1-phenylpropan-1-amine, and(S)—N,2-dimethyl-1-(pyridin-2-yl) propan-1-amine. (S)-2-amino-3-(oxazol-2-yl) propanoic acid, (S)-2-amino-3-(oxazol-5-yl) propanoic acid, (S)-2-amino-3-(1,3,4-oxadiazol-2-yl) propanoic acid, (S)-2-amino-3-(1,2,4-oxadiazol-3-yl) propanoic acid, (S)-2-amino-3-(5-fluoro-1H-indazol-3-yl) propanoic acid, (S)-2-amino-3-(1H-indazol-3-yl) propanoic acid, Ac-Nle; Ac-Arg; 7-Ahept; BzlSO₂, PyrPropHep; 2-Nac; Nba; Npa; Pba; Ppa; Tos; or a combination thereof; optionally wherein the N-terminus sequence comprises a string of 5 or 6 amino acids (G/L-G/L-G/L-G/L-G/L-G/L), each independently selected from Glu or Lys; or optionally wherein the N-terminus sequence comprises poly(glutamic acid) polypeptides (PGa), poly(aspartic acid) polypeptides (PAs), poly(lysine) polypeptides (PLy), poly(arginine) polypeptides (PAr), poly(histidine) polypeptides (PHi), poly(ornithine) polypeptides (POr), or combinations thereof (e.g., PLy-PGa-α-MSH);
wherein XAA1 is absent, comprises a Ser′ amino acid, or comprises a moiety selected from: D-Ser, NMe-Ser, Ile, Thr, Tyr, Tyr(Me), or D-stereoisomers thereof;
wherein XAA2 is absent, comprises a Tyr² amino acid, or comprises a moiety selected from: D-Tyr, Ile, 1-Nal, 2-Nal, 2-Pal, 3-Pal, D-Phe, NMe-Phe, D-NMe-Phe, D-hPhe, Arg, D-Arg, Bip, D-Bip, Cys, D-Cys, Dip, D-Dip, His, D-1-Nal, D-2-Nal, 2-Pal, 3-Pal, Phe(2—Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Ser, Thr, Tic, Tyr(Me), or D-stereoisomers thereof; a or Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof;
wherein XAA3 is absent, comprises a Ser³ amino acid, or comprises a moiety selected from: D-Ser, Ile, Leu, Nle, Tyr, Val, BrAc, or D-stereoisomers thereof, and optionally forms a cyclic peptide with suitable chemical groups at the 9, 10, 11 or 12 position;
wherein XAA4 is absent, comprises a Met⁴ amino acid, or comprises a moiety selected from: Lys, Lys(N3), BrAc, R4-R10, S4-S10, D-Met, Asp, Can, Cba, Cha, Cpna, Cpra, Cys, D-Cys, hCys, D-hCys, Glu, Gly, Hcy, Hle, Ile, Leu, (cyclohexyl)Gly, Nle, Nle(Met), Pen, D-Pen, Ser, Tyr, or Val, or D-stereoisomers thereof and optionally forms a cyclic peptide with suitable chemical groups at the 9, 10, 11 or 12 position;
wherein XAA5 is absent, comprises a Glu⁵ amino acid, or comprises a moiety selected from: D-Glu, Ala, Asn, Asp, Cys, D-Cys, hCys, α-Me-Cys, Dab, NDab, Dap, hGlu, Gln, Gly, Ile, Lys, D-Lys, Lys(N3), BrAc, R4-R10, S4-S10, NGlu, DabN3, Pra, OrnN3, Nle, Orn, Ser, Succ, Tyr, or 4-aminobutyric acid, or D-stereoisomers thereof and optionally forms a cyclic peptide with suitable chemical groups at the 9, 10, 11 or 12 position;
wherein XAA6 is absent, comprises a His⁶ amino acid, or comprises a moiety selected from: D-His, NMe-His, NMe-D-His, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Aib, Aic, Ala, D-Ala, Arg, Asn, Asp, Cha, Chg, Cit, Cys, D-Cys, Dab, Dap, Gln, Glu, His(1-Me), His(3-Me), Hyp, Hyp(Bzl), Ile, Leu, Lys, Met, Met(O), Met(O₂), Nle, D-Nle, Orn, 2-Pal, 3-Pal, 4-Pal, Phe, Pro, Sar, Ser, Ser(Bzl), Thr, Thr(OBzl), Tic, Tle, Trp, Tyr, Tyr(Me), or Val, cyclohexylglycine, cyclohexylalanine, tert-butylglycine, Gln(alkyl), Gln(aryl), Asn(alkyl), Asn(aryl), Tic, (2-pyridinyl)alanine, (3-pyridinyl)alanine, (4-pyridinyl)alanine, (2-thienyl)alanine, (3-thienyl)alanine, (4-thiazolyl)Ala, (2-furyl)alanine, (3-furyl)alanine, or a Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof and optionally forms a cyclic peptide with suitable chemical groups group at the 9, 10, 11 or 12 position;
wherein XAA7 is absent, comprises a Phe⁷ amino acid, or comprises a moiety selected from: D-Phe, NMe-Phe, D-NMe-Phe, D-hPhe, Arg, D-Arg, Bip, D-Bip, Cys, D-Cys, Dip, D-Dip, His, 1-Nal, D-1-Nal, 2-Nal, D-2-Nal, 2-Pal, 3-Pal, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Tic, Thi, D-Thi, Trp, Tyr, D-Tyr, Tyr(Me), D-Tyr(Me), Val, or D-Val, or D-stereoisomers thereof; or a Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof;
wherein XAA8 is absent, comprises a Arg⁸ amino acid, or comprises a moiety selected from: hArg, norArg, Agp, D-Arg, Arg (Me), Arg (Me)₂, Ala, Cys, D-Cys, Dab, Dap, Dpr (beta-Ala), Leu, Lys, hLys, Nle, (Nlys)Gly, NMe-Arg, Orn, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Ser, or Trp, or D-stereoisomers thereof; or Phe moiety optionally substituted independently by one or more of halogen (e.g., F, Cl, Br or I), hydroxyl, alkoxy, nitro, benzoyl, methyl, trifluoromethyl, amino, or cyano group, or D-stereoisomers thereof;
wherein XAA9 is absent, comprises a Trp⁹ amino acid, or comprises a moiety selected from: D-Trp, Trp(6-Me), Trp(7-Me), Aic, Atc, Ala, Arg, Asp, Bip, Cys, D-Cys, Cys-Trp, α-Me-Cys, Dab, 1-Nal, D-1-Nal, 2-Nal, D-2-Nal, NMe-Trp, Trp(Me), Tic, D-Tic, Tiq, D-Tiq, Tpi, or D-Tpi, R4-R10, S4-S10, Ndab, Orn or D-stereoisomers thereof and optionally forms a cyclic peptide with suitable chemical groups at the 3, 4 or 5 position;
wherein XAA10 is absent, comprises a Gly¹⁰ amino acid, or comprises a moiety selected from: D-Gly, Ala, D-Ala, Arg, 6-Ahx, Cys, D-Cys, Daa, Dab, DabN3, Glu, Lys, D-Lys, hLys, LysN3, Orn, Pra, Hpra. Trp, R4-R10, S4-S10, or 5-aminopentanoic acid, or D-stereoisomers thereof and optionally forms a cyclic peptide with group at the 3, 4 or 5 position;
wherein XAA11 is absent, comprises a Lys¹¹ amino acid, or comprises a moiety selected from: D-Lys, Ala, Asn, Asp, Cys, D-Cys, Dab, Glu, hGlu, Hgin, Gly, Lys(Me)₂, Pra, or Orn, or D-stereoisomers thereof and optionally forms a cyclic peptide with suitable chemical groups at the 3, 4 or 5 position;
wherein XAA12 is absent, comprises a Pro¹² amino acid, or comprises a moiety selected from: D-Pro, Ala, Asp, Cys, Gly, Ile, Leu, Met, Phe, Ser, Trp, Val, or D-stereoisomers thereof; or Phe moiety optionally substituted independently by one or more of halogen (e.g., F, Cl, Br or I), hydroxyl, alkoxy, nitro, benzoyl, methyl, trifluoromethyl, amino, or cyano groups, or D-stereoisomers thereof and optionally forms a cyclic peptide with suitable chemical groups at the 3, 4 or 5 position;
wherein XAA13 is absent, comprises a Val¹³ amino acid, or comprises a moiety selected from: D-Val, NMe-Val, Ala, Can, Cha, Cha, Cpna, Cpra, Cys, Hcy, Hle, Ile, Leu, Met, Nle, Pro, or D-stereoisomers thereof; and
wherein Y is absent, or comprises a C-terminus sequence selected from: NH2, OH, NH—CH₃; NH—CH₂—CH₃; NH—CH—(CH₃)₂, NH—CH₂—CH₂—CH₃, NH—CH₂—CH—(CH₃)₂, N(CH₃)₂, N(CH₂—CH₃)₂, OH, or Trp-NH₂.

In some embodiments, Z is Ac, PBA, octanoyl-PEG8G-G, PyAA, HyBA, HyPA, MoPA, HymBA, C7, C8, or C9; XAA1 is Ser or absent; XAA2 is Tyr or absent; XAA3 is Met or absent; XAA4 is Met or Nle or absent; XAA5 is Glu; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4—NH₂), D-Phe(4—NH₂), Phe(3,4-diF), Phe(4-F), D-Phe(4-F), Phe(4-Me), D-Phe(4-Me), Phe(4-OMe), D-Phe(4-OMe); XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Gly, Trp, or Ala; XAA11 is Lys, Gly, Asn, HgIn, Lys(Me)₂, or absent; XAA12 is Pro or absent; XAA13 is Val or absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 1. SEQ ID NO: 2, SEQ ID NO: 3, SEQ ID NO: 4, SEQ ID NO: 5, SEQ ID NO: 6, SEQ ID NO: 7, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 35.

In some embodiments, Z is Ac, PBA, octanoyl-PEG8G-G, PyAA, HyBA, HyPA. MoPA, HymBA, C7, C8, or C9; XAA1, XAA2, XAA3 are absent; XAA4 is Met or Nle or Lys(N3) or optionally Lys(N3) forms a cyclic peptide as a triazole group with Pra group at the 11 position; XAA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra, Lys(N3) or D-Pra group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe); XAA7 is Phe, D-Phe, NMe-Ph, D-NMe-Ph, Phe(2-Cl), D-Phe(2-Cl), Phe(2-F), D-Phe(2-F), Phe(2-Me), D-Phe(2-Me), Phe(2,4-diCl), D-Phe (2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Gly, Pra, Dab(N3), Hpra, Lys(N3) or D-Pra or optionally Pra. Dab(N3), Hpra, Lys(N3) or D-Pra forms a cyclic peptide as a triazole group with Lys(N3), D-Lys(N3), Dab(N3), Pra, or Orn(N3) group at the 10 position; XAA11 is absent, Pra or optionally Pra forms a cyclic peptide as a triazole group with Lys(N3) group at the 4 position; XAA12. XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 8, SEQ ID NO: 9, SEQ ID NO: 10, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 72, SEQ ID NO: 73, SEQ ID NO: 74, or SEQ ID NO: 75.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra, Lys(N3) or D-Pra group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe(2,4-diCl), D-Phe (2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe); XAA7 is Phe, D-Phe, NMe-Ph. D-NMe-Ph, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 5 position; XAA11, XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 8, SEQ ID NO: 9, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 72. SEQ ID NO: 73, SEQ ID NO: 74, or SEQ ID NO: 75.

In some embodiments, Z is Ac, PBA, octanoyl-PEG8G-G. PyAA, HyBA, HyPA, MoPA, HymBA, C7, C8, or C9; XAA1, XAA2, XAA3 are absent; XAA4 is Met or Nle; XAA5 is Glu, Lys(N3), D-Lys(N3), Dab(N3), Pra, or Orn(N3) or optionally Lys(N3), D-Lys(N3), Dab(N3), Pra, or Orn(N3) form a cyclic peptide as a triazole group with Pra, Dab(N3), Hpra. Lys(N3) or D-Pra group at the 10 position; XAA6 is His, NMe-His, Tyr. Tyr(Me), Phe, D-Phe, Phe(2-Cl), D-Phe(2-Cl), Phe(2-F), D-Phe(2-F), Phe(2-Me), D-Phe(2-Me), Phe(2,4-diCl), D-Phe (2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe); XAA7 is Phe, D-Phe, NMe-Ph. D-NMe-Ph, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Gly, Pra, Dab(N3), Hpra, Lys(N3) or D-Pra or optionally Pra, Dab(N3), Hpra, Lys(N3) or D-Pra forms a cyclic peptide as a triazole group with Lys(N3), D-Lys(N3), Dab(N3), Pra, or Orn(N3) group at the 5 position; XAA11 is Lys, Gly, Asn, HgIn, or Lys(Me) 2; XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, or SEQ ID NO: 71.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is R4, R5, R6, R7, R8. S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe); XAA7 is Phe, D-Phe, NMe-Ph. D-NMe-Ph, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4—NH₂), D-Phe(4—NH₂), Phe(3,4-diF), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 5 position; XAA11. XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequences include SEQ ID NO: 38, or SEQ ID NO: 39.

In some embodiments, Z is Ac. PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe); XAA7 is Phe, D-Phe, NMe-Ph. D-NMe-Ph, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4—NH₂), D-Phe(4—NH₂), Phe(3,4-diF), Phe(4-F), D-Phe(4-F), Phe(4-Me), D-Phe(4-Me), Phe(4-OMe), D-Phe(4-OMe), Tyr. Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is R4, R5, R6, R7, R8, S4, S5, S6, S7, S8 or optionally R4, R5, R6, R7, R8, S4, S5, S6, S7 or S8 form a cyclic peptide with R4, R5, R6, R7, R8, S4, S5, S6, S7 or S8 group at the 5 position; XAA11 is Lys, Gly, Asn, HgIn, or Lys(Me) 2; XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof.

In some embodiments, Z is Ac, PBA, octanoyl-PEG8G-G, PyAA, HyBA. HyPA, MoPA, HymBA, C7, C8, or C9; XAA1, XAA2, XAA3 are absent; XAA4 is Met or Nle; XAA5 is Cys or α-Me-Cys or optionally Cys or α-Me-Cys form a cyclic peptide with Cys or α-Me-Cys group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4-NH2), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe); XAA7 is Phe, D-Phe, NMe-Ph. D-NMe-Ph, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4-CF3), Phe(4-Cl), D-Phe(4-Cl), Phe(4—CN), D-Phe(4—CN), Phe(4—NH₂), D-Phe(4—NH₂), Phe(3,4-diF), Phe(4-F), D-Phe(4-F), Phe(4-Me), D-Phe(4-Me), Phe(4-OMe), D-Phe(4-OMe), Tyr, Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Cys or α-Me-Cys or optionally Cys or α-Me-Cys form a cyclic peptide with Cys or α-Me-Cys group at the 5 position; XAA11 is absent, Lys, Gly, Asn, HgIn, or Lys(Me) 2; XAA12. XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequence includes SEQ ID NO: 40.

In some embodiments, Z is Ac, PBA, octanoyl-PEG8G-G, PyAA, HyBA, HyPA, MoPA, HymBA, C7, C8, or C9; XAA1, XAA2, XAA3 are absent; XAA4 is Met or Nle; XAA5 is Dab or optionally Dab forms a cyclic peptide with Dab group at the 10 position; XAA6 is His, NMe-His, Tyr, Tyr(Me), Phe, D-Phe, Phe(2-Cl), D-Phe(2-Cl), Phe(2-F), D-Phe(2-F), Phe(2-Me), D-Phe(2-Me), Phe(2,4-diCl), D-Phe(2,4-diCl), Phe(2,4-diMe), D-Phe(2,4-diMe), Phe(3-Cl), D-Phe(3-Cl), Phe(3—CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4—CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe); XAA7 is Phe, D-Phe, NMe-Ph. D-NMe-Ph, Phe(2-Cl), D-Phe(2-Cl), Phe(2-F), D-Phe(2-F), Phe(2-Me), D-Phe(2-Me), Phe (2,4-diCl), D-Phe(2,4-diCl), Phe(2,4-diMe), D-Phe(2,4-diMe), Phe(3-Cl), D-Phe(3-Cl), Phe(3-CN), D-Phe(3—CN), Phe(4—NH₂), D-Phe(4—NH₂), Phe(3,4-diF), Phe(3-F), D-Phe(3-F), Phe(4-F), D-Phe(4-F), Phe(3-Me), D-Phe(3-Me), Phe(3,4-DiCl), D-Phe(3,4-DiCl), Phe(4—CF3), D-Phe(4-CF3), Phe(4-Cl), D-Phe(4-Cl), Phe(4—CN), D-Phe(4—CN), Phe(4-F), D-Phe(4-F), Phe(4-Me), D-Phe(4-Me), Phe(4-OMe), D-Phe(4-OMe), Tyr. Tyr(Me), D-Tyr, or D-Tyr(Me); XAA8 is Arg, NMe-Arg, Harg, or Arg(Me)2; XAA9 is Trp, NMe-Trp, or Trp(Me); XAA10 is Dab or optionally Dab forms a cyclic peptide with Dab group at the 5 position; XAA11 is absent, Lys, Gly, Asn, HgIn, or Lys(Me) 2; XAA12, XAA13 are absent; and Y is NH2 or absent. In some embodiments, XAA6 and XAA7 can each independently comprise a Phe moiety optionally substituted independently by one or more of halogen, hydroxyl, alkoxy, nitro, benzoyl, methyl, trifluoromethyl, amino, or cyano group, or D-stereoisomers thereof. In some embodiments, the specific amino acid sequence includes SEQ ID NO: 41.

In some embodiments, the α-MSH analogs include specific amino acid sequences. Such amino acid sequences may include any of those listed in Table 3 or fragments or variants thereof.

TABLE 3
α-MSH analog sequences

| ID | Sequence | SEQ ID NO: |
|---|---|---|
| KR-001 | Ac-Tyr-Ser-Met-Glu-His-Phe-Arg-Trp-Gly-Lys-Pro-Val-NH2 | 1 |
| KR-002 | Ac-Ser-Met-Glu-His-Phe-Arg-Trp-Gly-Lys-Pro-Val-NH2 | 2 |
| KR-003 | Ac-Met-Glu-His-Phe-Arg-Trp-Gly-Lys-Pro-Val-NH2 | 3 |
| KR-004 | Ac-Glu-His-Phe-Arg-Trp-Gly-Lys-Pro-Val-NH2 | 4 |
| KR-005 | Ac-Ser-Tyr-Ser-Met-Glu-His-Phe-Arg-Trp-Gly-Lys-Pro-NH2 | 5 |
| KR-006 | Ac-Ser-Tyr-Ser-Met-Glu-His-Phe-Arg-Trp-Gly-Lys-NH2 | 6 |
| KR-007 | Ac-Glu-His-Phe-Arg-Trp-Gly-Lys-Pro-NH2 | 7 |
| KR-008 | Ac-Nle-[Lys(N3)-His-Phe-Arg-Trp-Pra]-NH2 | 8 |
| KR-009 | Ac-Nle-[Lys(N3)-His-DPhe-Arg-Trp-Pra]-NH2 | 9 |
| KR-010 | Ac-[Lys(N3)-Glu-His-Phe-Arg-Trp-Gly-Pra]-NH2 | 10 |
| KR-011 | Cyclo(-Ac-Glu-His-Phe-Arg-Trp-Cys)-NH2 | 11 |
| KR-012 | Cyclo(-Ac-Glu-His-Phe-Arg-Trp-Gly-Cys)-NH2 | 12 |
| KR-013 | Ac-R5-Glu-His-Phe-Arg-Trp-Gly-S5-NH2 | 13 |
| KR-014 | Ac-S5-Glu-His-Phe-Arg-Trp-Gly-S5-NH2 | 14 |
| KR-015 | Ac-R8-Glu-His-Phe-Arg-Trp-Gly-S5-NH2 | 15 |
| KR-016 | PBA-Ser-Tyr-Ser-Met-Glu-His-Phe-Arg-Trp-Gly-Lys-Pro- | 16 |
| | Val-NH2 | |
| KR-017 | Octanoyl-PEG8-G-G-Ser-Tyr-Ser-Met-Glu-His-Phe-Arg-Trp- | 17 |
| | Gly-Lys-Pro-Val-NH2 | |
| KR-018 | PyAA-Ser-Tyr-Ser-Met-Glu-His-Phe-Arg-Trp-Gly-Lys-Pro- | 18 |
| | Val-NH2 | |
| KR-019 | HyBA-Ser-Tyr-Ser-Met-Glu-His-Phe-Arg-Trp-Gly-Lys-Pro- | 19 |
| | Val-NH2 | |
| KR-020 | Octanoyl-Ser-Tyr-Ser-Met-Glu-His-Phe-Arg-Trp-Gly-Lys- | 20 |
| | Pro-Val-NH2 | |
| KR-021 | Ac-Ser-Tyr-Ser-Met-Glu-(NMe-His)-Phe-Arg-Trp-Gly-Lys- | 21 |
| | Pro-Val-NH2 | |
| KR-022 | Ac-Ser-Tyr-Ser-Met-Glu-His-(NMe-Phe)-Arg-Trp-Gly-Lys- | 22 |
| | Pro-Val-NH2 | |
| KR-023 | Ac-Ser-Tyr-Ser-Met-Glu-His-Phe-(NMe-Arg)-Trp-Gly-Lys- | 23 |
| | Pro-Val-NH2 | |
| KR-024 | Ac-Ser-Tyr-Ser-Met-Glu-His-Phe-Arg-(NMe-Trp)-Gly-Lys- | 24 |
| | Pro-Val-NH2 | |
| KR-025 | Ac-Ser-Tyr-Ser-Met-Glu-His-(D-NMePhe)-Arg-Trp-Gly-Lys- | 25 |
| | Pro-Val-NH2 | |
| KR-026 | Ac-Nle-R5-His-DPhe-Arg-S5-Trp-NH2 | 26 |
| KR-027 | Ac-Nle-cyc(NGlu-His-DPhe-Arg-Dab)-Trp-NH2 | 27 |
| KR-028 | Ac-Nle-cyc(Glu-His-DPhe-Arg-NDab)-Trp-NH2 | 28 |
| KR-029 | Ac-Nle-cyc(NGlu-His-DPhe-Arg-NDab)-Trp-NH2 | 29 |
| KR-030 | Ac-Nle-cyc(NDab-His-DPhe-Arg-NDab)-Trp, L: succinic | 30 |
| | acid-NH2 | |
| KR-031 | Ac-Nle-cyc(aMe-Cys-His-DPhe-Arg-aMe-Cys)-Trp-NH2 | 31 |
| KR-032 | Ac-Nle-cyc(Cys-His-DPhe-Arg-Cys)-Trp, L: m-xylene-NH2 | 32 |
| KR-033 | Ac-R8-Glu-His-DPhe-Arg-Trp-Gly-S8-NH2 | 33 |
| KR-034 | Ac-Nle-R8-His-Dphe-Arg-Trp-S5-Gly-NH2 | 34 |
| KR-035 | Ac-Nle-Glu-His-Phe-Arg-Trp-Gly-Lys-NH2 | 35 |
| KR-036 | Cyclo(-Ac-Nle-Glu-His-Phe-Arg-Trp-Gly-Lys-Cys)-NH2 | 36 |
| KR-037 | Ac-Nle-[Lys(N3)-His-DPhe-Arg-Trp-Pra]-Lys-NH2 | 37 |
| KR-038 | Ac-Nle-[R5-His-DPhe-Arg-Trp-S5]-NH2 | 38 |
| KR-039 | Ac-Nle-[S5-His-DPhe-Arg-Trp-S5]-NH2 | 39 |
| KR-040 | Ac-Nle-[Cys-His-DPhe-Arg-Trp-Cys] Linker: a,a'-Dibromo- | 40 |
| | m-xylene-NH2 | |
| KR-041 | Ac-Nle-[Dab-His-DPhe-Arg-Trp-Dab] Linker: succinic acid- | 41 |
| | NH2 | |
| KR-042 | Ac-Nle-[Dab(N3)-His-DPhe-Arg-Trp-Dab(N3)] linkr: 3,3'- | 42 |
| | Oxybis[1-propyne]-NH2 | |
| KR-043 | Ac-Nle-[Lys(N3)-NMeHis-DPhe-Arg-Trp-Pra]-NH2 | 43 |
| KR-044 | Ac-Nle-[Lys(N3)-His-(NMe-Dphe)-Arg-Trp-Pra]-NH2 | 44 |
| KR-045 | Ac-Nle-[Lys(N3)-His-DPhe-NMeArg-Trp-Pra]-NH2 | 45 |
| KR-046 | Ac-Nle-[Lys(N3)-His-DPhe-Arg-NMeTrp-Pra]-NH2 | 46 |
| KR-047 | Ac-Nle-[Lys(N3)-Phe(4-NH2)-DPhe-Arg-Trp-Pra]-NH2 | 47 |
| KR-048 | Ac-Nle-[Lys(N3)-His-Dphe(4-CF3)-Arg-Trp-Pra]-NH2 | 48 |
| KR-049 | Ac-Nle-[Lys(N3)-His-Dphe(4-F)-Arg-Trp-Pra]-NH2 | 49 |
| KR-050 | Ac-Nle-[Lys(N3)-His-DPhe-NMeArg-NMeTrp-Pra]-NH2 | 50 |
| KR-051 | Ac-Nle-[Lys(N3)-NMeHis-DPhe-Arg-NMeTrp-Pra]-NH2 | 51 |
| KR-052 | Ac-Nle-[Lys(N3)-NMeHis-DPhe-NMeArg-Trp-Pra]-NH2 | 52 |
| KR-053 | HyBA-Nle-[Lys(N3)-His-DPhe-Arg-Trp-Pra]-Lys-NH2 | 53 |
| KR-054 | Ac-Nle-[Lys(N3)-His-DPhe-HArg-Trp-Pra]-Lys-NH2 | 54 |
| KR-055 | Ac-Nle-[Lys(N3)-His-DPhe-Arg(Me)2-Trp-Pra]-Lys-NH2 | 55 |
| KR-056 | Ac-Nle-[Lys(N3)-His-DPhe-Arg-Trp-Pra]-Asn-NH2 | 56 |
| KR-057 | Ac-Nle-[Lys(N3)-His-DPhe-Arg-Trp-Pra]-Hgln-NH2 | 57 |
| KR-058 | Ac-Nle-[Lys(N3)-His-DTyr(Me)-Arg-Trp-Pra]-Lys-NH2 | 58 |
| KR-059 | Ac-Nle-[Lys(N3)-His-DPhe-Arg-Trp-Pra]-Lys(Me)2-NH2 | 59 |
| KR-060 | Ac-Nle-[Lys(N3)-His-DPhe-Arg-Trp(Me)-Pra]-Lys-NH2 | 60 |
| KR-061 | Ac-Nle-[Lys(N3)-Phe(OCH3)-DPhe-Arg-Trp-Pra]-Lys-NH2 | 61 |
| KR-062 | HyPA-Nle-[Lys(N3)-His-DPhe-Arg-Trp-Pra]-Lys-NH2 | 62 |
| KR-063 | MoPA-MoPA-Nle-[Lys(N3)-His-DPhe-Arg-Trp-Pra]-Lys-NH2 | 63 |
| KR-064 | HymBA-HymBA-Nle-[Lys(N3)-His-DPhe-Arg-Trp-Pra]-Lys-NH2 | 64 |
| KR-065 | Ac-Nle-[Orn(N3)-His-DPhe-Arg-Trp-Pra]-Lys-NH2 | 65 |
| KR-066 | Ac-Nle-[Lys(N3)-His-DPhe-Arg-Trp-HPra]-Lys-NH2 | 66 |
| KR-067 | Ac-Nle-[Pra-His-DPhe-Arg-Trp-Lys(N3)]-Lys-NH2 | 67 |
| KR-068 | Ac-Nle-[Orn(N3)-His-DPhe-Arg-Trp-Hpra]-Lys-NH2 | 68 |
| KR-069 | Ac-Nle-[DLys(N3)-His-DPhe-Arg-Trp-Pra]-Lys-NH2 | 69 |
| KR-070 | Ac-Nle-[Lys(N3)-His-DPhe-Arg-Trp-DPra]-Lys-NH2 | 70 |
| KR-071 | HyBA-Nle-[Lys(N3)-His-Dphe(4-F)-Arg-Trp-Pra]-Hgln-NH2 | 71 |
| KR-072 | HyBA-Nle-[Lys(N3)-His-Dphe(4-F)-Arg-Trp-Pra]-NH2 | 72 |
| KR-073 | HyBA-Nle-[Lys(N3)-His-DPhe-Arg-Trp-Pra]-NH2 | 73 |
| KR-074 | PyAA-Nle-[Lys(N3)-His-DPhe-Arg-Trp-Pra]-NH2 | 74 |
| KR-075 | HyBA-Nle-[Lys(N3)-NMeHis-Dphe(4-F)-Arg-NMeTrp-Pra]-NH2 | 75 |
| R-1 | Ac-Ser-Tyr-Ser-Met-Glu-His-Phe-Arg-Trp-Gly-Lys-Pro-Val- | 76 |
| | NH2 | |
| R-2 | Ac-Ser-Tyr-Ser-Nle-Glu-His-DPhe-Arg-Trp-Gly-Lys-Pro-Val- | 77 |
| | NH2 | |
| R-3 | Ac-Ser-Tyr-Ser-Met-Glu-His-Phe-Arg-Trp-Gly-Lys-Pro-Val- | 78 |
| | Gly-Lys-Lys-Arg-Arg-Pro-Val-Lys-Val-Tyr-Pro-Asn-Gly-Ala- | |
| | Glu-Asp-Glu-Ser-Ala-Glu-Ala-Phe-Pro-Leu-Glu-Phe-NH2 | |
| R-4 | Ac-Nle-Cyc(Glu-His-Dphe-Arg-Dab)-Trp-NH2 | 79 |

In some embodiments, the α-MSH analogs comprise a peptide portion conjugated to a non-peptide-based portion. In some embodiments, the non-peptide-based portion is selected from: lipids, small molecules, RNA, DNA, polymers, or combinations thereof. In some embodiments, the α-MSH analogs comprise a peptide portion conjugated to one or more of: a cholesterol oleate moiety, a cholesteryl laurate moiety, an α-tocopherol moiety, a phytol moiety, an oleate moiety, an unsaturated cholesterol-ester moiety, or a lipophilic compound selected from acetanilides, anilides, aminoquinolines, benzhydryl compounds, benzodiazepines, benzofurans, cannabinoids, cyclic peptides, dibenzazepines, digitalis glycosides, ergot alkaloids, flavonoids, imidazoles, quinolines, macrolides, naphthalenes, opiates (such as, but not limited to, morphinans or other psychoactive drugs), oxazines, oxazoles, phenylalkylamines, piperidines, polycyclic aromatic hydrocarbons, pyrrolidines, pyrrolidinones, stilbenes, sulfonylureas, sulfones, triazoles, tropanes, and vinca alkaloids. In some embodiments, the α-MSH analogs comprise a peptide portion conjugated to one or more of: polyalkylene oxide homopolymers, polypropylene glycols, polyoxyethylenated polyols and copolymers thereof, polyethylene glycol (PEG), an albumin binding moiety, or a cell penetrating moiety. In some embodiments, the α-MSH analogs comprise a peptide portion conjugated to fatty acids, phospholipids, or sterols. In some embodiments, the α-MSH analogs comprise a peptide portion conjugated to one or more of: Palm-PEG8-G-G-Ser-Tyr (SEQ ID NO: 169); Ac—K(Palm)-G-G-Ser-Tyr (SEQ ID NO: 170), capric acid (C10), lauric acid (C12), myristic acid (C14), palmitic acid (C16), stearic acid (C18), PEG4, or PEG8.

### Synthesis

The present disclosure presents methods of synthesizing peptides and compounds of the present disclosure, including α-MSH peptide analogs and α-MSH analog compounds. In some embodiments, peptides and compounds of the present disclosure can be obtained by inducing the formation of a covalent bond between an amino group at the N-terminus of a peptide (if provided), and a carboxyl group of a reactive amino acid side chain moiety (if provided). In some embodiments, peptides and compounds of the present disclosure can be synthesizing by any known conventional procedure for the formation of a peptide linkage between amino acids. Such conventional procedures include, for example, any solution phase procedure permitting a condensation between the free alpha amino group of an amino acid or residue thereof (having its carboxyl group or other reactive groups protected) and the free primary carboxyl group of another amino acid or residue thereof (having its amino group or other reactive groups protected). In some embodiments, the peptides of the present disclosure may be synthesized by solid-phase synthesis and purified according to methods known in the art. Any of a number of well-known procedures utilizing a variety of resins and reagents may be used to prepare the peptides of the present disclosure.

In some embodiments, the process for synthesizing peptides may be carried out by a procedure whereby each amino acid in the desired sequence is added one at a time in succession to another amino acid or residue thereof. In some embodiments, the process for synthesizing peptides may be carried out by a procedure whereby multiple peptide fragments with portions of the desired amino acid sequence are first synthesized, and then condensed to provide the desired peptide sequence.

In some embodiments, the process for synthesizing peptides may be carried out using solid phase peptide synthesis, which includes methods well known and practiced in the art (e.g., Symphony Multiplex Peptide Synthesizer (Rainin Instrument Company) automated polypeptide synthesizer). In some embodiments, the process for synthesizing peptides may be carried using standard Fmoc methodology on an automated synthesizer (e.g., Advanced ChemTech 440M05, Louisville, Ky). In some embodiments, the process for synthesizing peptides may be carried using coupling reagents such as 2-(1-H-Benzotriazol-1-yl)-1,1,3,3-tetramethyluronium hexafluorophosphate (HBTU) and/or 1-Hydroxybenzotriazole (HOBt).

Solid phase peptide synthesis can be carried out by sequentially incorporating the desired amino acid residues one at a time into the growing tide chain according to the general principles of solid phase methods. These methods are disclosed in numerous references, including Merrifield, et al., Solid phase synthesis (Nobel lecture), Angew Chem (1985) 24:799-810; Barany et al., The Peptides, Analysis, Synthesis and Biology, Vol. 2; Gross et al., Eds. Academic Press 1-284 (1980), the contents of which are each incorporated herein by reference in their entirety, as related to processes and protocols for synthesizing peptides.

Solid phase synthesis of the peptide is generally commenced from the C-terminal end of the peptide by coupling a protected alpha amino acid to a suitable resin. Examples of known methods for preparing substituted amide derivatives on solid-phase have been described in the art (see, e.g., Barn D. R. et al., Tetrahedron Letters (1996), 37:3213-3216; DeGrado et al., J. Org. Chem., (1982) 47:3258-3261; the contents of which are each incorporated herein by reference in their entirety as related to methods and systems for solid-phased peptide synthesis). As an example, starting materials can be prepared by attaching an alpha amino-protected amino acid by an ester linkage to a p-benzyloxybenzyl alcohol (Wang) resin or an oxime resin by well-known means. The peptide chain is grown with the desired sequence of amino acids, and the peptide-resin is then treated with a solution of appropriate amine (such as methyl amine, dimethyl amine, ethylamine, and so on). Peptides employing a p-benzyloxybenzyl alcohol (Wang) resin may be cleaved from the resin by aluminum chloride in DCM, and peptides employing an oxime resin may be cleaved by DCM.

In some embodiments, reactive side chain groups of the various amino acid residues are protected with suitable protecting groups, which prevent a chemical reaction from occurring at that site until the protecting group is removed. In some embodiments, the alpha amino group of an amino acid residue or fragment is protected while that entity reacts at the carboxyl group, followed by the selective removal of the alpha amino protecting group to allow a subsequent reaction to take place at that site. Examples of protecting groups for use in the present disclosure have been disclosed and are known in solid phase synthesis methods and solution phase synthesis methods.

In some embodiments, alpha amino groups may be protected by a suitable protecting group, including: a urethane-type protecting group, such as benzyloxycarbonyl (Z) and substituted benzyloxycarbonyl, such as p-chlorobenzyloxycarbonyl, P-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz); aliphatic urethane-type protecting groups, such as t-butyloxycarbonyl (Boc), diisopropylmethoxycarbonyl, isopropoxycarbonyl, and allyloxycarbonyl.

In some embodiments, guanidino amino groups (such as those found in arginine) may be protected by a suitable protecting group, such as nitro, p-toluenesulfonyl (Tos), Z, pentamethylchromanesulfonyl (Pmc), adamantyloxycarbonyl, pentamethyldihydrobenzofuran-5-sulfonyl (Pbf) and Boc.

As one not-limiting example, solid phase synthesis of a peptide can be commenced from the C-terminal end of the peptide by coupling a protected alpha amino acid to a suitable resin. The starting material can be prepared by attaching an alpha amino-protected amino acid by an ester linkage to a p-benzyloxybenzyl alcohol (Wang) resin, a 2-chlorotrityl chloride resin or an oxime resin, by an amide bond between an Fmoc-Linker, such as p-[(R,S)-α-[1-(9H-fluor-en-9-yl)-methoxyformamido]-2,4-dimethyloxybenzyl]-phenoxyacetic acid (Rink linker) to a benzhydrylamine (BHA) resin, or by other means well known in the art. Fmoc-Linker-BHA resin supports are commercially available and generally used when feasible. The resins are then carried through repetitive addition cycles as necessary to add amino acids sequentially. The alpha amino Fmoc protecting groups are then removed under basic conditions (e.g., Piperidine, piperazine, diethylamine, or morpholine (20-40% v/v) in N,N-dimethylformamide (DMF)). Following removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. After the peptide is synthesized, if desired, the orthogonally protected side chain protecting groups may be removed using methods well known in the art for further derivatization of the peptide.

Reactive groups in a peptide can be selectively modified, either during solid phase synthesis or after removal from the resin. For example, peptides can be modified to obtain N-terminus modifications, such as acetylation, while on resin, or may be removed from the resin by use of a cleaving reagent and then modified. Similarly, methods for modifying side chains of amino acids are well known to those skilled in the art of peptide synthesis. The choice of modifications made to reactive groups present on the peptide will be determined, in part, by the characteristics that are desired in the peptide.

In some embodiments, the N-terminus group is modified by introduction of an N-acetyl group. As a non-limiting example, the peptide synthesis can include a step wherein, after removal of the protecting group at the N-terminal, a resin-bound peptide is reacted with acetic anhydride in dichloromethane in the presence of an organic base, such as diisopropylethylamine. Other methods of N-terminus acetylation are known in the art, including solution phase acetylation.

In some embodiments, peptides of the present disclosure can comprise cyclic peptides having one or more bridging moieties (e.g., cyclic structure, staple, bridge, etc.).

In some embodiments, the peptide can be synthesized using solid phase peptide synthesis, and then cyclized prior to cleavage from the peptide resin. If the peptide is being cyclized through reactive side chain moieties, the desired side chains are first deprotected under specific deprotection conditions in a suitable solvent, and a cyclic coupling agent is then added. Suitable solvents include, but are not limited to: DMF, dichloromethane (DCM), and 1-methyl-2-pyrrolidone (NMP). Suitable cyclic coupling reagents include, but are not limited to: 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), benzotriazole-1-yl-oxy-tris(dimethylamino)phosphoniumhexafluorophosphate (BOP), benzotriazole-1-yl-oxy-tris(pyrrolidino) phosphoniumhexafluorophosphate (PyBOP), 2-(7-aza-1H-benzotriazol-1-yl)-1,1,3,3-tetramethyluronium tetrafluoroborate (TATU), 2-(2-oxo-1 (2H)-pyridyl)-1,1,3,3-tetramethyluronium tetrafluoroborate (TPTU), and N,N′-dicyclohexylcarbodiimide/1-hydroxybenzotriazole (DCCI/HOBt). In some embodiments, coupling of the cyclic moiety to the peptide chain is initiated by use of a suitable base, such as N,N-diisopropylethylamine (DIPEA), sym-collidine, or N-methylmorpholine (NMM).

The cyclized peptides can then be cleaved from the solid phase using any suitable reagent, such as ethylamine in DCM. The resulting crude peptide is dried, and remaining amino acid side chain protecting groups (if any) are cleaved using suitable reagents, such as trifluoroacetic acid (TFA) in the presence of water and 1,2-ethanedithiol (EDT). The final product is precipitated by adding cold ether and collected by filtration. Final purification can be by reverse phase high performance liquid chromatography (RP-HPLC), using a suitable column, such as a C18 column. Other methods of separation or purification, such as methods based on the size or charge of the peptide, can also be employed. Once purified, the peptide can be characterized by any number of methods, such as high-performance liquid chromatograph (HPLC), amino acid analysis, mass spectrometry, and the like.

In some embodiments, peptides of the present disclosure can comprise one or more modifications (e.g., substitution, addition, deletion) to one or more terminus (e.g., N-terminus, C-terminus, or both) of the peptide sequence. In some embodiments, terminus-modified peptides can be synthesized using solid phase peptide synthesis, and then modified prior to cleavage from the peptide resin.

### Cell Therapy Product

In some embodiments, the compositions of the disclosure may include cell therapy products. Cell therapy product: As used herein, a “cell therapy product” refers to cells or a population of cells that have been propagated, expanded, selected, pharmacologically treated, genetically engineered or otherwise altered ex vivo in their biological characteristics, to be administered to a subject in need thereof.

In certain aspects, the inventions and compositions disclosed herein comprise a cell therapy product administered in combination with one or more α-MSH peptides or α-MSH analog. For example, in certain embodiments, the methods and compositions disclosed herein contemplate the co-administration of a cell therapy product (e.g., one or more isolated ocular stem cells, mammalian primitive retinal stem cells or retinal ganglion precursor cells) and one or more α-MSH peptides and/or α-MSH analogs. Alternatively, and in lieu of such a co-administration, such a cell therapy product may be engineered to express an α-MSH peptides, as further disclosed herein. For example, in certain aspects an isolated ocular stem cell, mammalian primitive retinal stem cells or a retinal ganglion precursor cell may be engineered to express an α-MSH peptide.

Cell therapy products may include one or more cell types such as, but not limited to, stem cells, progenitor cells, and/or somatic cells. Progenitor and/or somatic cells described herein may be derived by the differentiation of the stem cells. Non-limiting examples of cell therapy products may include hematopoietic cells, mesenchymal cells, embryonic cells, and umbilical cord blood cells, dendritic cell vaccines, activated T or B lymphocytes, monocytes, and modified or unmodified cancer cells, allogeneic pancreatic islet cells, chondrocytes for cartilage repair, keratinocytes, fibroblasts, and/or hepatocytes.

### Tissues

In some embodiments, cell therapy products may include one or more tissues. There are four basic types of tissue including connective tissues, epithelial tissue, muscle tissue, and nervous tissue. Connective tissue supports other tissues and binds them together (bone, blood, and lymph tissues). Epithelial tissue provides a covering (skin, the linings of the various passages inside the body). Muscle tissue includes striated (also called voluntary) muscles that move the skeleton, and smooth muscle, such as the muscles that surround the stomach. Nerve tissue is made up of nerve cells (neurons) and is used to carry “messages” to and from various parts of the body.

### Stem Cells

In some embodiments, cell therapy products may include stem cells. Stem cells as used herein refers to an undifferentiated cell of a multicellular organism that is capable of giving rise to indefinitely more cells of the same type, and from which certain other kinds of cell arise by differentiation. Stem cells can differentiate into specialized cells and can divide (through mitosis) to produce more stem cells. They are found in multicellular organisms. In mammals, there are two broad types of stem cells: embryonic stem cells, which are isolated from the inner cell mass of blastocysts, and adult stem cells, which are found in various tissues. In adult organisms, stem cells and progenitor cells act as a repair system for the body, replenishing adult tissues. In a developing embryo, stem cells can differentiate into all the specialized cells, such as ectoderm, endoderm and mesoderm, but also maintain the normal turnover of regenerative organs, such as blood, skin, or intestinal tissues.

Pluripotent Stem Cells (PSCs) and Induced Pluripotent Stem Cells (iPSCs)

In some embodiments, cell therapy products may include pluripotent stem cells (PSCs). Generally, PSCs are a group of cells that can maintain an undifferentiated state indefinitely and can differentiate into most cells of the body. In some embodiments, cell therapy products may include induced pluripotent stem cells (iPSCs). iPSC refers to a type of pluripotent stem cell that can be artificially derived from a non-pluripotent cell, typically an adult somatic cell, by inducing a forced expression of certain genes and transcription factors.

### Mesenchymal Stem Cells (MSCs)

In some embodiments, cell therapy products may include mesenchymal stem cells (MSCs). Generally, mesenchymal stem cells are of stromal origin and may differentiate into a variety of tissues and cell types, including: osteoblasts (bone cells), chondrocytes (cartilage cells), myocytes (muscle cells) adipocytes (fat cells). MSCs have been isolated from placenta, adipose tissue, lung, bone marrow and blood, Wharton's jelly from the umbilical cord and teeth (perivascular niche of dental pulp and periodontal ligament). MSCs have ability to differentiate, provide trophic support, and modulate innate immune response.

### Adult Stem Cells (ASCs)

In some embodiments, cell therapy products may include adult stem cells (ASCs). Adult stem cells are undifferentiated cells, found throughout the body after development, that multiply by cell division to replenish dying cells and regenerate damaged tissues. Also known as somatic stem cells, they can be found in juvenile as well as adult animals and human bodies. Adult stem cells have the ability to divide or self-renew indefinitely, and generate all the cell types of the organ from which they originate, potentially regenerating the entire organ from a few cells. The production of adult stem cells does not require the destruction of an embryo. Additionally, when adult stem cells are obtained from the intended recipient (an autograft) there is no risk of immune rejection.

### Cancer Stem Cells (CSCs)

In some embodiments, cell therapy products may include cancer stem cells (CSCs). Cancer stem cells (CSCs) are cancer cells (found within tumors or hematological cancers) that possess characteristics associated with normal stem cells, specifically the ability to give rise to all cell types found in a particular cancer sample. CSCs are a subpopulation of tumor cells that can drive tumor initiation and can cause relapses. At the time point of tumor initiation, CSCs originate from either differentiated cells or adult tissue resident stem cells. Several biomarkers that characterize CSCs have been identified and correlated to diagnosis, therapy and prognosis.

### Hematopoietic Stem Cells

In some embodiments, cell therapy products may include hematopoietic stem cells. Hematopoietic stem cells are found in the bone marrow and umbilical cord blood and give rise to all the blood cell types.

### Progenitor Cells

In some embodiments, cell therapy products may include progenitor cells. A progenitor cell is to a biological cell that, like a stem cell, has a tendency to differentiate into a specific type of cell, but is already more specific than a stem cell. While stem cells can replicate indefinitely, progenitor cells can divide only a limited number of times. They are descendants of stem cells that then further differentiate to create specialized cell types.

There are many types of progenitor cells throughout the human body. Each progenitor cell is only capable of differentiating into cells that belong to the same tissue or organ. Some progenitor cells have one final target cell that they differentiate to, while others have the potential to terminate in more than one cell type. In some embodiments, the progenitor cell is a neural progenitor cell, a liver progenitor cell, corneal epithelial progenitor cells, a photoreceptor progenitor cell, a monoblast cell, myeloblast cell, or a hematopoietic progenitor cell.

### Somatic Cells

In some embodiments, cell therapy products may include somatic cells. A somatic cell is any biological cell forming the body of a multicellular organism other than a gamete, germ cell, gametocyte or undifferentiated stem cell. In humans, somatic cells are diploid, i.e., containing two sets of chromosomes, one inherited from each parent. DNA mutations in somatic cells can affect an individual, but they cannot be passed on to their offspring.

### Immune Cells

In some embodiments, cell therapy products may include one or more immune cells. An immune cell is a cell that is part of the immune system and helps the body fight infections and other diseases. Immune cells develop from stem cells in the bone marrow and become different types of white blood cells. These include neutrophils, eosinophils, basophils, mast cells, monocytes, macrophages, dendritic cells, and lymphocytes (natural killer cells (NK cells), B cells and T cells).

### T Cells Including Tregs

In some embodiments, cell therapy products may include one or more T cells. T cells are part of the immune system and develop from stem cells in the bone marrow. They help protect the body from infection and may help fight cancer. T cells may also be referred to herein as T lymphocyte or thymocyte. T cells can be distinguished from other lymphocytes by the presence of a T-cell receptor (TCR) on their cell surface. Groups of specific, differentiated T cell subtypes have a variety of important functions in controlling and shaping the immune response.

One function of T cells is immune-mediated cell death, and it is carried out by two major subtypes of T cells: CD8+ “killer” and CD4+ “helper” T cells. CD8+ T cells, also known as “killer T cells”, are cytotoxic, i.e., they are able to directly kill virus-infected cells, as well as cancer cells. CD8+ T cells are also able to use small signaling proteins (e.g., cytokines) to recruit other types of cells when mounting an immune response. CD4+ T cells, function as “helper cells”. Unlike CD8+ killer T cells, the CD4+ helper T (T_{H}) cells activate memory B cells and cytotoxic T cells, which leads to a larger immune response. The specific adaptive immune response regulated by the TH cell depends on its subtype, which is distinguished by the types of cytokines they secrete.

### NK Cells

In some embodiments, cell therapy products may include one or more natural killer cells. A natural killer cell is a type of immune cell or lymphocyte that can also be referred to as a NK cell or a large granular lymphocyte (NK-LGL) cell. NK cells are unique in that they have the ability to recognize and kill stressed cells (e.g., tumor cells or virus-infected cells) in the absence of antibodies and the major histocompatibility complex (MHC), allowing for a much faster immune reaction. NK cells are known to differentiate and mature in the bone marrow, lymph nodes, spleen, tonsils, and thymus, where they then enter into the circulation.

### B Cells

In some embodiments, cell therapy products may include one or more B cells. A B cell, also referred to as B lymphocytes, are a type of white blood cell. B cells product antibody molecules which may be either secreted or inserted into the plasma membrane where they served as a part of B-cell receptors. B cells, unlike the other two classes of lymphocytes, T cells and NK cells, express B cell receptors (BCRs) on their cell membrane. BCRs allow the B cell to bind to a foreign antigen, against which it will initiate an antibody response.

B cells develop from hematopoietic stem cells (HSCs) that originate from bone marrow. B cells undergo two types of selection, positive selection and negative selection, while developing in the bone marrow to ensure proper development, both involving B cell receptors (BCR) on the surface of the cell. Immature B cells may migrate from the bone marrow into the spleen and then transition into mature or naïve B cells.

### Monocytes

In some embodiments, cell therapy products may include one or more monocytes. A monocyte is a type of leukocyte or white blood cell that can differentiate into macrophages and dendritic cells. Monocytes also influence adaptive immune responses and exert tissue repair functions. In general, monocytes and their macrophage and dendritic cell progeny serve three main functions in the immune system: phagocytosis, antigen presentation, and cytokine production.

### Granulocytes

In some embodiments, cell therapy products may include one or more granulocytes. Granulocytes, also referred to as polymorphonuclear leukocytes, are cells in the innate immune system characterized by the presence of specific granules in their cytoplasm. There are four types of granulocytes: basophils, eosinophils, neutrophils, and mast cells. Granulocytes are derived from stem cells residing in the bone marrow.

### Fibroblasts

In some embodiments, cell therapy products may include one or more fibroblasts. A fibroblast is a type of biological cell that synthesizes the extracellular matrix and collagen, produces the structural framework for animal tissues, and plays a critical role in wound healing. In addition, fibroblasts can play a critical role in immune response to a tissue injury and in immune regulation through tumor-associated host fibroblasts (TAF)-derived extracellular matrix (ECM) components and modulators. The fibroblasts from different anatomical sites in the body express many genes that code for immune mediators and proteins. These mediators of immune response enable the cellular communication with hematopoietic immune cells.

### Chondrocytes

In some embodiments, cell therapy products may include one or more chondrocytes. A chondrocyte is a cell found in healthy cartilage that produces and maintains the cartilaginous matrix, which consists mainly of collagen and proteoglycans. Chondrocytes proliferate and secrete extracellular matrix to maintain and sustain the cartilage and they can response to outside stimuli and tissue damage. In some aspects, chondrocytes may be responsible for degenerative conditions, such as osteoarthritis. Chondrocytes are differentiated from mesenchymal stem cells and can undergo terminal differentiation when they become hypertrophic.

### Keratinocytes

In some embodiments, cell therapy products may include one or more keratinocytes. A keratinocyte is the primary type of cell found in the epidermis, the outermost layer of the skin. Keratinocytes form a barrier against environmental damage by heat, UV radiation, water loss, pathogenic bacteria, fungi, parasites, and viruses. Pathogens invading the upper layers of the epidermis can cause keratinocytes to produce proinflammatory mediators, particularly chemokines, such as CXCL10 and CCL2 (MCP-1), which attract monocytes, natural killer cells, T-lymphocytes, and dendritic cells to the site of the pathogen invasion. Keratinocytes differentiate from epidermal stem cells in the lower part of the epidermis and migrate towards the surface, finally becoming corneocytes and eventually are shed off, which happens every 40 to 56 days in humans.

### Hepatocytes

In some embodiments, cell therapy products may include one or more hepatocytes. A hepatocyte is a cell of the main parenchymal tissue of the liver and hepatocytes make up about 80% of the liver's mass. Hepatocytes play a role in: protein storage; metabolism; synthesis of cholesterol, bile salts and phospholipids; detoxification, modification, and excretion of exogenous and endogenous substances; initiation of formation and secretion of bile; and protein synthesis, and can also activate innate immunity against invading microorganisms by secreting innate immunity proteins.

### Pancreatic Islet Cells

In some embodiments, cell therapy products may include one or more pancreatic islet cells. Pancreatic islets also rereferred to as islets of Langerhans are groups of cells located in the pancreas. Pancreatic islets are the regions of the pancreas that contain the endocrine (hormone-producing) cells, which include alpha cells (glucagon producing), beta cells (insulin producing), delta cells (somatostatin producing), epsilon cells (ghrelin producing), and pancreatic polypeptide cells (pancreatic polypeptide producing). Pancreatic islets additionally contain stromal cells, vascular cells, immune cells, and neural cells.

### MCR Expressing Cells

In some embodiments, cell therapy products may include one or more melanocortin receptor (MCR) expressing cells. MCRs are a family of five seven-transmembrane G-protein coupled receptors. As discussed above, MCRs and corresponding melanocortic peptides have also been found to mediate a number of physiological conditions, including: immunomodulation, motivation, learning, memory, behaviour, inflammation, body temperature, pain, perception, blood pressure, heart rate, vascular tone, brain blood flow, nerve growth, placental development, aldosteron synthesis and release, thyroxin release, spermatogenesis, ovarian weight, prolactin and FSH secretion, uterine bleeding in women, sebum and pheromone secretion, blood glucose levels, weight homeostasis, and intrauterine fetal growth (as well as other events surrounding parturition).

### Ocular Cells

In some embodiments, cell therapy products may include ocular cells, such as ocular stem cells. The ocular system includes the eye and its visual system (e.g., cornea, lens, and fluids). Ocular cells may include ocular stem cells, ocular cell-fate further restricted precursor cells, such as retinal ganglion precursors, photoreceptor precursors, immature RPE cells, and corneal endothelial precursors. In some embodiments, the cells are isolated. In certain embodiments, the cells are selected from the group consisting of isolated ocular stem cells, mammalian primitive retinal stem cells or retinal ganglion precursor cells.

In some embodiments, one or more α-MSH peptide analogs (e.g., SEQ ID NOs: 1-79) are administered (e.g., administered ophthalmically or topically to the subject) as a pre-treatment to a subject, and the subject is subsequently treated with ocular cells (e.g., ocular stem cells). For example, a subject may be pre-treated with one or more α-MSH peptide analogs, following which are population of ocular stem cells may be administered to or transplanted intraocularly in the subject. In such embodiments, pretreatment with one or more α-MSH peptide analogs prior to the administration or transplantation of a population of ocular stem cells confer cytoprotective properties and enhance the survival of the subsequently administered or transplanted ocular stem cells in the subject. In still other embodiments, the α-MSH peptide analogues disclosed herein are co-administered with the ocular stem cells. Pre-treatment with one or more α-MSH peptide analogs prior to the administration or transplantation of a population of ocular stem cells creates a niche suitable for receiving the subsequently administered or transplanted ocular stem cells in the subject.

### Engineered Cells

In some embodiments, cell therapy products may include one or more engineered cells. An engineered cell may be a cell that has been modified in some manner. Examples of engineered cells include cells engineered using one or more gene editing methods known to those of skill in the art, including CRISPR, TALENS, Zinc Finger, etc. In one aspect, an engineered cell is a cell modified to express a chimeric antigen receptor (CAR).

In certain embodiments, ocular cells (e.g., ocular stem cells) may be engineered to endogenously express a transgene encoding a polypeptide (e.g., an α-MSH peptide), and such engineered cells present a new approach to facilitating sustainable drug delivery, and may provide an efficient means of delivering an α-MSH peptide. In certain aspects, such engineered ocular cells (e.g., ocular stem cells) are used for cell-mediated delivery of an α-MSH peptide, and provide compositions and methods that are useful for the ocular cell-mediated delivery of an α-MSH peptide into the intraocular tissues of a subject, thereby providing a safe, effective, and sustainable release of such an α-MSH peptide to such subject's eye.

In certain embodiments, the ocular cells disclosed herein comprise ocular stem cells. In certain embodiments, the ocular cells comprise ocular cell-fate further restricted precursor cells, such as retinal ganglion precursors, photoreceptor precursors, immature RPE cells, and corneal endothelial precursors, all as further described in Zhao, et al., Invest Ophthalmol Vis Sci. 2016 Dec. 1; 57 (15): 6878-6884, the contents of which are included by reference herein in their entirety. In certain embodiments, the ocular cell comprises an ocular cell-fate further restricted precursor cell. In certain embodiments, the ocular cell-fate further restricted precursor cell comprises a photoreceptor precursor cell. In certain embodiments, the ocular cell-fate further restricted precursor cell comprises a retinal ganglion precursor cell. In yet other embodiments, the ocular cell-fate further restricted precursor cell comprises a retinal pigmented epithelial (RPE) cell. In certain embodiments, the ocular cell-fate further restricted precursor cell comprises a corneal endothelial cell. In other embodiments, the ocular cell comprises the differentiated progenies of ocular stem cells.

Also disclosed herein are compositions and methods useful for the sustainable ocular cell-mediated intraocular delivery (e.g., ocular stem cell-mediated delivery) of an α-MSH peptide for the treatment of eye diseases or conditions. Some embodiments of the present inventions include methods of sustainable ocular cell-mediated intraocular delivery (e.g., ocular stem cell-mediated delivery) of an α-MSH peptide into a subject's eye in need of treatment of an ocular disease or disorder. In certain aspects, such methods comprises a step of (a) preparing a drug expression construct that contains an α-MSH peptide coding sequence (e.g., encoding an α-MSH peptide) that is linked to a promoter, wherein the α-MSH polypeptide is linked to a leader sequence (e.g., a leader sequence comprising or selected from the group consisting of SEQ ID NO: 171, SEQ ID NO: 172, SEQ ID NO: 173, SEQ ID NO: 174, SEQ ID NO: 175 and/or SEQ ID NO: 176, shown in Table 4 below); (b) introducing the expression construct comprising or otherwise based upon a viral vector (e.g., an adeno-associated viral vector or lentiviral vector) or a mammalian plasmid expression vector into ocular cells (e.g., one or more ocular cells selected from the group consisting of ocular stem cells, photoreceptor precursors, retinal ganglion precursors, immature RPE cells and/or corneal endothelial cells) in vitro to form engineered ocular cells that can express an α-MSH peptide; (c) formulating engineered ocular cells that are able to produce or express an α-MSH peptide into a therapeutic composition; (d) injecting the therapeutic composition comprising the engineered ocular cells into the vitreous, the anterior chamber, the subretinal space, and/or the suprachoroidal space of the subject's eye; and (e) causing the production and secretion of the expressed an α-MSH peptide from the engineered ocular cells in a sustainable manner into the surrounding ocular tissues of the subject, and thereby providing a sustained therapeutic effect for treating the subject's ocular disease or disorder. In certain aspects, the expression construct comprises a sequence encoding an α-MSH peptide. For example, in certain embodiments, expression of an α-MSH peptide coding sequence is driven by the human eukaryotic translation elongation factor 1 alpha1 short form promoter (EFS) in the mammalian gene expression lentiviral vector, and comprises a mammalian gene expression lentiviral vector.

Many leader sequences may be used in connection with the compositions and methods disclosed herein. Exemplary leader sequences include Interleukin-2 (IL2 leader) (e.g., SEQ ID NO: 171), IGFBP (e.g., SEQ ID NO: 172), VEGF-A (e.g., SEQ ID NO: 173), Vitronectin (e.g., SEQ ID NO: 174), Albumin (e.g., SEQ ID NO: 175), and/or Complement Factor H (e.g., SEQ ID NO: 176). In some embodiments, the leader sequence is IL2 signal peptide.

TABLE 4
Leader Sequences

| | | SEQ |
|---|---|---|
| ID | Sequence | ID NO: |
| IL2 | MYRMQLLSCIALILALVTNS | 171 |
| IGFBP | MLPRVGCPALPLPPPPLLPLLLLLLG | 172 |
| | ASGGGGGARA | |
| VEGF-A | MNFLLSWVHWSLALLLYLHHAKWSQA | 173 |
| Vitronectin | MAPLRPLLILALLAWVALA | 174 |
| Albumin | MKWVTFISLLFLFSSAYS | 175 |
| Complement | MRLLAKIICLMLWAICVA | 176 |
| Factor H | | |

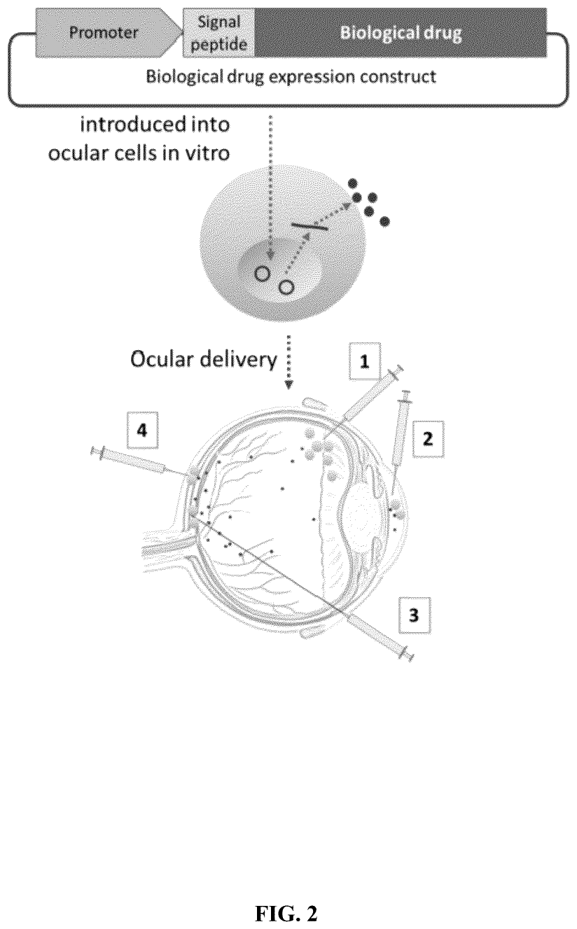

In some embodiments, the promoter is a constitutively active promotor. In some embodiments, the expression vectors (e.g., plasmids, AAV, and lentiviral vectors which have been used for gene therapy in clinical settings) may be used as a promoter to transduce the authentic ocular stem cells. The vector contains a biological factor coding sequence lead by a signal peptide sequence. For example, an expression cassette of IL2 leader sequence fused to an α-MSH peptide coding sequence (open reading frame) may be constructed, as depicted in FIG. 2.

An α-MSH peptide can be expressed in the engineered ocular stem cells, and the expressed α-MSH peptide then can be released from or secreted by the engineered ocular stem cells. Once the engineered ocular stem cells containing an α-MSH peptide are injected into a subject's eye, the transplanted cells not only can continuously release such α-MSH peptide, but also provide trophic support or nutrition to rescue retinal visual cells that are degenerating in a subject's eye.

In some embodiments, the engineered ocular stem cells may be transplanted to the subretinal space of the macula and surrounding areas, where the transplanted ocular stem cells then integrate into the retinal structure and do three different things. First, such transplanted ocular stem cells release an α-MSH peptide directly into the macular region to block the invasion of blood vessels. Second, such transplanted cells serve as a durable reservoir of trophic or nutritive factors to rescue the dying retinal cells in the area. Third, such transplanted cells can differentiate into new retinal pigment epithelium (RPE) and retinal visual cells and repair the visual circuitry necessary for sight.

To achieve a long-term release of an α-MSH peptide without the burden of repeated injections, for example, a transgene of an α-MSH peptide is expressed under the control of a ubiquitous promoter such as EF1alpha core promoter sequence identified in Table 5 (SEQ ID NO: 177). A human IL2 signal peptide is placed at the N-terminus of an α-MSH peptide. The viral expression vectors can be used to deliver the transgene into the PSC-derived ocular cells. These vectors may include replication-deficient recombinant adeno-associated viral vectors as well as lentiviral vectors. Plasmids or non-viral expression vectors may also be utilized. Since the α-MSH peptide are expressed in human ocular cells, it is expected that they will likely have full human post-translational modifications.

TABLE 5
Promotor Sequences

| | | SEQ ID |
|---|---|---|
| ID | Sequence | NO: |
| EF1 | ggctccggtgcccgtcagtgggcagagcgcacatcgc | 177 |
| alpha | ccacagtccccgagaagttggggggaggggtcggcaa | |
| | ttgatccggtgcctagagaaggtggcgcggggtaaac | |
| | tgggaaagtgatgtcgtgtactggctccgcctttttc | |
| | ccgaggggggggagaaccgtatataagtgcagtagtc | |
| | gccgtgaacgttctttttcgcaacgggtttgccgcca | |
| | gaacacagg | |

Also disclosed herein are therapeutic compositions for treating ocular diseases or disorders (e.g., neovascular AMD) in a human subject in need thereof, such compositions comprising ocular cells that are introduced with a transgene expression vector in vitro, wherein the transgene expression vector comprises cis-regulatory and promoter sequences that control the expression of a transgene encoding an α-MSH peptide; and wherein the ocular cells comprising the transgene are formulated into a suspension of cells for intraocular administration to the human subject. In certain embodiments the transgene expression vector is an AAV or lentiviral vector. In certain embodiments, the ocular stem cells comprising the transgene are then cryopreserved for long-term storage and, such cryopreserved ocular stem cells may be subsequently thawed and formulated into a suspension of cells for intraocular administration to the human subject. In certain aspects, the ocular cells comprise ocular stem cells. In certain embodiments, the ocular cells comprise an ocular cell-fate further restricted precursor cell (e.g., an ocular cell-fate further restricted precursor cell selected from the group consisting of a photoreceptor precursor cell, a retinal ganglion precursor cell, a retinal pigmented epithelial (RPE) cell, a corneal endothelial cell, and any combinations thereof). In certain embodiments, the ocular cell comprises a cell-fate restricted progeny of an ocular stem cell.

Certain embodiments of the present inventions are directed to ocular cells comprising the isolated mammalian ocular stem cells (OSCs) or primitive retinal stem cells (pRSCs), isolated mammalian retinal ganglion precursor cells (RGPCs), isolated mammalian photoreceptor precursor cell (PRPCs), isolated mammalian retinal pigment epithelial cells (RPECs), or isolated mammalian corneal endothelial cells (CECs). These ocular cells can be produced by in vitro methods, for example, as described in Zhao, et al., WO 2015/054526, U.S. Pat. No. 10,220,117 B2, and WO 2017/190136 A1, all of which are incorporated by reference in their entirety herein. For example, in certain embodiments, the ocular cells comprise isolated mammalian pRSCs and can be produced and isolated by: (a) culturing isolated pluripotent stem cells (PSCs) (e.g., embryonic stem cells (ESCs) or induced pluripotent stem cells (iPSCs)) from a mammal in a cell culture medium that is free of feeder cells, feeder-conditioned medium or serum so as to produce and grow a culture of the isolated PSCs; and (b) contacting the culture of the isolated PSCs so grown with one or more of an inhibitor for Wnt or TGF-β/BMP signaling so as to differentiate the isolated PSCs of (a) into primitive retinal stem cells, thereby producing isolated mammalian pRSCs, as described in Zhao, et al., WO 2015/054526. In certain embodiments, the ocular cells comprise isolated mammalian RGPCs, which can be produced and isolated by: (a) culturing isolated primitive retinal stem cells (pRSCs) from a mammal in a cell culture medium that is free of feeder cells, feeder-conditioned medium or serum so as to produce and grow a culture of the isolated pRSCs; and (b) contacting the culture of the isolated pRSCs so grown with one or more of an inhibitor of Wnt, Notch, or FGFR/VEGFR signaling so as to differentiate the isolated pRSCs into isolated RGPCs, thereby producing isolated mammalian RGPCs, as also described in Zhao, et al., WO 2015/054526.

In addition, in certain embodiments the mammalian retinal progenitor cells and corneal endothelial cells are directly isolated from donor tissues, for example, fetal retina and cornea donor tissues. These ocular cells can be used as a suitable vehicle and durable reservoir to deliver an α-MSH peptide to a diseased eye of a subject. Like pluripotent stem cells (PSCs) and other tissue stem cells, such as mesenchymal stem cells (MSCs), ocular stem/progenitor and precursor cells normally express low levels of immunogenicity markers. In addition, the eye is a well-known immune privileged organ. Recent data from a Phase 2b clinical trial of intravitreal administration of jCells (human fetal retinal progenitor cells) for RP treatment without immunosuppression drug also indicates the safety of ocular cells for sustainable drug delivery (see, Kuppermann, B., “Intravitreal Injection of Allogeneic Human Retinal Progenitor Cells (Jcell) for Treatment of Retinitis Pigmentosa,” The Retina Society). Accordingly, in certain aspects, the compositions and methods disclosed herein are characterized by their low immunogenicity and, for example, may be administered to a patient without immunosuppression and/or the administration of or pre-treatment with one or more immunomodulating agents.

In certain embodiments, the α-MSH peptide is or comprises a protein (e.g., a protein encoded by the amino acid sequence comprising SEQ ID NO: 76). In certain embodiments, the α-MSH peptide is a biologically active fragment or variant of the α-MSH peptide.

In certain embodiments, the inventions disclosed herein are generally directed to therapeutic compositions and related methods for treating an ocular disease or disorder in a subject (e.g., a human subject) in need thereof. In certain embodiments, such methods comprise a step of contacting one or more isolated ocular cells (e.g., ocular stem cells) with a transgene expression vector in vitro, wherein the transgene expression vector comprises cis-regulatory and promoter sequences that control the expression of a transgene encoding a polypeptide of an α-MSH peptide; wherein the ocular stem cells comprising the transgene are formulated into a suspension of cells for intraocular administration to the human subject. In certain embodiments, expression of an α-MSH peptide coding sequence may be driven by the human eukaryotic translation elongation factor 1 alpha1 short form promoter (EFS) in the mammalian gene expression lentiviral vector.

In some embodiments, the ocular cells comprise one or more immature corneal endothelial cells (CECs) or corneal endothelial precursor cells (CEPCs), which are derived from ocular stem cells. For example, as described in Zhao, et al., WO 2017/190136 A1, the immature CEC or CEPC induction was driven by small molecules and in a stepwise fashion of lineage specification. During the initial phase, PSC fate was restricted to the eye field-like state and became eye field stem cell (EFSC). In the second phase, PSC-derived EFSC was further directed toward either neural crest lineage or retinal lineage. The CECs were directly induced from ocular neural crest stem cells (NCSCs) by suppressing TGF-beta and ROCK signaling. As disclosed herein, the CECs or CEPCs can be engineered (e.g., transfected with one or more expression constructs) and used as a vehicle to deliver the expressed α-MSH peptide to the anterior chamber of the subject's eye for the treatment of corneal diseases.

Also disclosed herein are engineered ocular cell lines, wherein isolated cells of such engineered ocular cell line endogenously express a transgene encoding an α-MSH peptide, and wherein the cells comprise an edited genome that results in the endogenous expression of the transgene compared to a control cell line. In certain embodiments, the α-MSH peptide comprises SEQ ID NO: 76. In certain embodiments, the ocular cell line comprises ocular stem cells. In yet other embodiments, the ocular cell line comprises cell-fate further restricted precursors of ocular stem cells (e.g., ocular cell-fate further restricted precursors of ocular stem cells selected from the group consisting of photoreceptor precursor cells, retinal ganglion precursor cells, retinal pigmented epithelial (RPE) cells, corneal endothelial cells, and combinations thereof). In certain embodiments, the inventions disclosed herein are directed to methods of treating an ocular disease or disorder in a human subject (e.g., neovascular AMD) in need thereof, comprising administering one or more of engineered ocular cells disclosed herein to a subject (e.g., a human subject).

Some embodiments include the ocular stem cell-mediated sustainable intraocular α-MSH peptide delivery approach that can provide safe, effective, and sustainable release of an α-MSH peptide in a patient's eye for treating various eye diseases or disorders. This new approach may be used to treat a broad spectrum of eye diseases and conditions, regardless of the underlying disease-causing genetic mutation(s).

In certain aspects, also disclosed are engineered ocular cell lines and related methods of treatment comprising administering such engineered ocular cell lines to a subject. For example, upon the intravitreal administration of cells from such engineered ocular cell lines to a subject, such cells endogenously express a transgene encoding an α-MSH peptide and secrete such peptide to thereby cause a sustainable ocular cell-mediated intraocular delivery of such peptide (e.g., delivery of such polypeptide into a subject's eye in need of treatment of an ocular disease or disorder with such polypeptide).

As used herein, the term “cell line” refers to a clonal population of cells (e.g., engineered ocular stem cells) that are able to continue to divide and not undergo senescence. In certain embodiments, cells of the engineered ocular cell line endogenously express a transgene encoding an α-MSH peptide, wherein the cells comprise an edited genome that results in the endogenous expression of the transgene, for example, compared to a control cell line. As used herein, the term “control cell line” generally refers to a cell line that is genetically similar to an engineered ocular cell line, but has not been engineered in the same way. For example, an engineered ocular cell line may express an endogenous transgene when compared to a control ocular cell line that is not engineered in the same way. In certain embodiments, the engineered ocular cell line may express an endogenous transgene encoding an α-MSH peptide or a biologically active fragment or variant thereof.

In certain embodiments, the ocular cell line comprises ocular stem cells. In certain embodiments, the engineered ocular cell line comprises cell-fate further restricted precursors of ocular stem cells. For example, in certain aspects the ocular cell-fate further restricted precursors of ocular stem cells comprise or are selected from the group consisting of a photoreceptor precursor cell, a retinal ganglion precursor cell, a retinal pigmented epithelial (RPE) cell, a corneal endothelial cell and combinations thereof.

Ocular Stem Cells from iPSCs

Cell therapy represents a tremendously promising approach by replenishing the injured tissues with healthy cells, regardless of underlying genetic or acquired cause. Substantial evidence suggests that ocular progenitor and precursor cells could be used as potential drug products to treat vision loss (see, Wang Y, et al., Cell Death Dis. 2020 Sep. 23; 11(9):793. doi: 10.1038/s41419-020-02955-3. PMID: 32968042; PMCID: PMC7511341). Preclinical studies demonstrated that the transplantation of these cells into the eye could result in photoreceptor replacement and significant slowing of host photoreceptor loss. A subset of the progenitors developed into mature neurons, including presumptive photoreceptors expressing recoverin, rhodopsin, or cone opsin. Rescue of cells in the outer nuclear layer (ONL), along with widespread integration of donor cells into the inner retina of recipient mice, showed improved light-mediated behavior compared with the control animals (see, Semo M, et al., Trans Vis Sci Tech. 2016; 5 (4): 6; and Wang Z, et al., Med Sci Monit. 2020 Mar. 28; 26:0921184. Doi: 10.12659/MSM.921184. PMID: 32221273; PMCID: PMC7139196).

In addition, the cell therapy approach has been clinically validated for retinal and corneal care. In early phases of clinical trials, subjects treated with healthy fetal retinal progenitor cells (RPCs) or corneal endothelial cells (CECs) expanded from a donor tissue have experienced significant and durable improvements in visual acuity. The treatment of intravitreally delivered RPCs was well-tolerated, safe, and efficacious in halting and even reversing the vision loss among RP patients. Notably, no immunosuppression drug was used in the RPC cell therapy studies. It represents a significant advantage for progenitor cells over more matured cell products, which often require immune suppression. However, relying on fetal donor tissue causes concerns on the limitation of source material, scalability of manufacture, and ethical conflicts. Thus, there is a significant clinical need for an alternative authentic ocular cell source.

During the early stages of embryonic head development, the eye primordium or eye field is formed under the influence of WNT and BMP signaling gradients. The eye field cells are gradually restricted to different ocular lineages and cell fates. Remarkably, this eye development process can be mimicked in vitro by the differentiation of human pluripotent stem cells (ESCs or iPSCs) through a targeted and stepwise process. The present inventors have previously developed a highly efficient, small-molecule-based method to induce ocular stem cells from iPSCs in vitro under a defined set of culture conditions, as further described in Zhao, et al., WO 2015/054526, the contents of which are incorporated by reference in their entirety).

In some embodiments, ocular stem cells (OSCs) (or eye field stem cells (EFSCs), or primitive retinal stem cells (pRSCs)) are produced in vitro by the method described in Zhao, et al., WO2015/054526, which comprises (1) culturing isolated pluripotent stem cells (PSCs) from a mammal in a cell culture medium that is free of feeder cells, feeder-conditioned medium or serum so as to produce and grow a culture of the isolated PSCs; and (2) contacting the culture of the isolated PSCs with one or more of an inhibitor for Wnt or TGF-β/BMP signaling so as to differentiate the isolated PSCs into OSCs, thereby producing isolated mammalian OSCs. In some embodiment, one or more of an inhibitor for Wnt or TGFβ/BMP signaling is a combination of inhibitors for Wnt and TGF-β/BMP signaling. The culture of isolated PSCs may be an adherent culture. In some embodiment, the culture of the isolated PSCs is a monolayer culture. In some embodiment, the culture of isolated PSCs is grown to near confluence before contacting with one or more of an inhibitor for Wnt or TGFβ/BMP signaling or a combination of inhibitors for Wnt and TGF-β/BMP signaling. In some embodiments, an inhibitor for Wnt or TGFβ/BMP signaling is a small molecule inhibitor. The induced pluripotent stem cells (iPSCs) from a mammal may be similarly treated in place of isolated PSCs to produce isolated mammalian OSCs. In some embodiments, the isolated mammalian OSCs are directed to differentiate toward specific retinal cell fates in vitro using small molecule inducers of differentiation. In some embodiments, the specific retinal cell fates include neuroretinal cells and non-neuronal cells. In some embodiments, the neuroretinal cells include retinal ganglion precursor cells (RGPCs) and photoreceptor precursor cells (PRPCs). In some embodiment, the non-neuronal cells include retinal pigment epithelial cells (RPECs).

The isolated mammalian retinal ganglion precursor cells (RGPCs) may be produced by an in vitro method comprising: (a) culturing isolated primitive retinal stem cells (pRSCs) from a mammal in a cell culture medium that is free of feeder cells, feeder-conditioned medium or serum so as to produce and grow a culture of the isolated pRSCs; and (b) contacting the culture of the isolated pRSCs so grown with one or more of an inhibitor of Wnt, Notch, or FGFR/VEGFR signaling so as to differentiate the isolated pRSCs into isolated mammalian RGPCs, thereby producing isolated mammalian RGPCs.

The isolated mammalian photoreceptor precursors from isolated mammalian pRSCs may be produced by an in vitro method comprising: (a) culturing and growing dissociated pRSCs from a mammal in a neural induction medium comprising one or more of an inhibitor of a TGF-β/Activin receptor-like kinases ALK-4,-5 or -7, glycogen synthase kinase-3 (GSK-3), Notch or Wnt signaling or an activator of a hedgehog signaling for a sufficient time to induce pRSCs to a photoreceptor cell lineage fate without visible morphological changes or expression of photoreceptor-specific markers; and (b) followed by, culturing and growing pRSCs of step a) in neural induction medium comprising retinoic acid or taurine or both so as to differentiate the mammalian pRSCs to photoreceptor precursors, thereby producing isolated mammalian photoreceptor precursors; wherein the culture medium is free of feeder cells, feeder-conditioned medium or serum.

The non-neural isolated mammalian retinal pigment epithelial cells (RPECs) may be produced by an in vitro method comprising: (a) culturing pRSCs from a mammal in culture medium comprising nicotinamide or activin A or both in absence of SMAD signaling inhibitor for a sufficient time so as to direct pRSCs toward RPE fate; and (b) culturing the pRSCs in culture medium comprising one or more of a N1 medium supplement, taurine, hydrocortisone, or triiodo-thyronine; so as to differentiate the mammalian pRSCs to mammalian RPECs, thereby, producing isolated mammalian RPECs, wherein the medium is free of feeder cells or feeder-conditioned medium.

Such isolated mammalian OSCs, which can be produced by the described method (see, Zhao, et al, WO 2015/054526) in sufficient quantity and quality, are suitable for cellular transplantation or grafting to an eye of a subject without a need for cellular fractionation or cellular purification prior to cellular transplantation or grafting.

These cells have characteristics of eye field stem cells and can be directed to differentiate toward retinal and corneal lineages in responding to the inductive cues provided in culture. This novel process paves the way to develop off-the-shelf iPSC-derived authentic ocular cell products, which have the advantages of unlimited supply, consistent quality, and no ethical concerns.

Because ocular stem/progenitor and precursor cells are highly expandable and amenable to genetic modifications, they are suitable for rescue and replacing the damaged visual cells and may serve as cellular vehicle to deliver an α-MSH polypeptide to diseased eyes. Ocular fate-restricted cells are of particular interest in this context, as they have been shown to survive for extended periods of time after injection into the vitreous of eyes and to provide retina protection activity on its own. The phase 2b study sponsored by jCyte was designed to evaluate the safety and efficacy of an investigational treatment for RP patients that injected jCells (allogeneic fetal RPCs) intravitreally. Up to 6 million cells were dosed in a single injection. The treatment was well-tolerated, safe, and efficacious in halting, and even reversing, the vision loss among RP patients. Notably, immunosuppression drug was not used in the study (see, Kuppermann, B., “Intravitreal Injection of Allogeneic Human Retinal Progenitor Cells (Jcell) for Treatment of Retinitis Pigmentosa,” The Retina Society). It represents a significant advantage for using progenitor cells over more matured cell products that often require immune suppression.

Two primary considerations in developing an appropriate cell-based therapy are the source of therapeutic cells and the immunological consequences following transplantation. Potential sources of therapeutic differentiated retinal pigmented epithelium (RPE) cells for transplantation studies include pluripotent stem cells (PSCs) derived from embryonic, fetal, or adult cell sources. Recently, the results from early clinical trials of retinal degeneration cell therapy based on four kinds of stem/progenitor cells-RPCs, ESCs, iPSCs, and mesenchymal stem cells (MSCs), suggest that RPCs seem to be the best candidate for the treatment, since it is efficacious and has a shallow risk of rejection and tumorigenesis (Wang Y, et al., Cell Death Dis. 2020 Sep. 23; 11(9):793. doi: 10.1038/s41419-020-02955-3. PMID: 32968042; PMCID: PMC7511341). However, using fetal donor retina as the source of RPCs has some significant concerns on the limitation of source, scalability and process consistency of manufacture, and ethical conflicts.

The anterior chamber of the eye and the brain have long been described as “immune privileged” sites in transplantation. This phenomenon could have contributed to the tolerance to intraocular transplantation of allogeneic progenitor cells. Besides, cultured murine neural progenitors did not express major histocompatibility complex (MHC) class I or class II antigens. They were tolerated as allografts even following transplantation to the kidney capsule, a conventional (non-privileged) site.

Although there were some safety concerns about the clinical application of iPSCs due to the potential risk of integration of transgenes into the genome of reprogrammed cells in early years, more recently, small molecules, plasmid vectors, or non-integrating viruses have been utilized for iPSC reprogramming to avoid genomic insertions, thus reducing the risk for translational application and more relevant for clinical applications. The allogeneic iPSC approach could also reduce the cost of iPSC-based cell therapy. It opens a new avenue of retinal disease treatment that could be generally safe, physiologically stable, highly cost-efficient, and targeted at retinal cell revival and regeneration.

The iPSC-derived ocular stem cells can be extensively expanded in culture. These cells, which in analogy to continuously self-renewing embryonic stem cells, can differentiate into retinal neurons and retinal pigment epithelial (RPE) cells in vitro and after transplantation into the retina, and give rise to ocular neural crest stem cells (oNCSCs) and corneal endothelial cells (CECs) in vitro. These cells provide a sustainable source of trophic support for dysfunctional ocular tissues. They may be used as the suitable vehicle and durable reservoir to deliver therapeutic products such as nucleic acids, proteins and other drug moieties to the diseased eye. An advantage of the present inventions' use of the ocular progenitor and precursor cells is that these cells do not need encapsulation to protect themselves from the host immune system.

Sustainable an α-MSH Peptide Delivery Approach where Corneal Endothelial Cells are Used for Cell-Mediated Delivery

In some embodiments, corneal endothelial cells (CECs), which can also be derived from ocular stem cells (OSCs) or primitive retinal stem cells (pRSCs) (see, Zhao, et al., WO 2015/054526), may be used as a vehicle to deliver an α-MSH peptide to the anterior chamber of the eye for the treatment of corneal diseases. The method of induction of corneal endothelial cells from human pluripotent stem cells have been previously described (see, Zhao, et al, WO 2017/190136A1, the contents of which are incorporated herein in their entirety).

In some embodiments, for the treatment of corneal endothelial diseases, the corneal endothelial cells (CECs) containing an α-MSH peptide can be injected into the anterior chamber. This cell therapy approach has been clinically validated for corneal care. In early research and clinical trials in Japan, subjects treated with healthy CECs expanded from a donor cornea have experienced significant and durable improvements in key measures of corneal health including visual acuity, corneal endothelial cell density, and corneal thickness (Kinoshita S, et al., N Engl J Med. 2018 Mar. 15; 378(11):995-1003. Doi: 10.1056/NEJMoa1712770. PMID: 29539291; and Numa K, et al., Ophthalmology. 2021 April; 128(4):504-514. doi: 10.1016/j.ophtha.2020.09.002. Epub 2020 Sep. 6. PMID: 32898516). One potential application is to treat neovascular cornea, for example, by administration of CECs or iPSC-derived corneal endothelial cells transduced with a viral vector encoding an α-MSH peptide in the anterior chamber.

Corneal Endothelial Cells Derived from Ocular Stem Cells

Corneal endothelial cells (CECs) can be prepared in vitro using the method described in Zhao, et al., WO 2017/190136 A1. Eye cell fate specification was carried out under defined small molecule-driven conditions and in a stepwise fashion of lineage specification. During the initial phase, PSC fate is restricted to the eye field-like state and becomes ocular stem cell (OSC). In the second phase, PSC-derived OSC is further committed toward retinal lineage or ocular neural crest lineage. The formation of CEnC sheet is directly induced from ocular neural crest stem cell (oNCSC) by suppressing TGF-beta and ROCK signaling in the culture. The induction of oNCSC is initiated by promoting WNT signaling in OSC. Within two weeks of induction, the majority of cells express the typical neural crest markers p75NTR and HNK-1. OSC-derived oNCSCs can be propagated and cryopreserved. Finally, CEC monolayer sheet formation is induced from adherent oNCSCs in the presence of a ROCK inhibitor. The polygonal shaped CEC-like cells become visible after a week in culture. The expression of typical CEC markers such as ZO-1, N-cadherin, and Na+/K+-ATPase is also detected.

Some embodiments of the present inventions include methods of sustainable ocular cell-mediated intraocular delivery of an α-MSH peptide into a subject's eye in need of treatment of an ocular disease or disorder with an α-MSH polypeptide. In certain aspects, such methods comprises a step of (a) preparing an α-MSH polypeptide expression construct that contains an α-MSH peptide coding sequence that is linked to a promoter, wherein an α-MSH polypeptide is linked to a leader sequence; (b) introducing the expression construct comprising or otherwise based upon a viral vector (e.g., an adeno-associated viral vector or lentiviral vector) or a mammalian plasmid expression vector into ocular cells (e.g., one or more ocular stem cells selected from the group consisting of ocular stem cells, photoreceptor precursors, retinal ganglion precursors, immature RPE cells and/or corneal endothelial cells) in vitro to form engineered ocular cells that can express the α-MSH peptide; (c) formulating engineered ocular cells that are able to produce or express a therapeutic composition; (d) injecting the therapeutic composition comprising the engineered ocular cells into the vitreous, the anterior chamber, the subretinal space, and/or the suprachoroidal space of the subject's eye; (e) causing the production and secretion of the expressed α-MSH peptide from the engineered ocular cells in a sustainable manner into the surrounding ocular tissues of the subject, and thereby providing a sustained therapeutic effect for treating the subject's ocular disease or disorder.

Certain embodiments of the present inventions are directed to ocular cells comprising the isolated mammalian ocular stem cells (OSCs) or primitive retinal stem cells (pRSCs), isolated mammalian retinal ganglion precursor cells (RGPCs), isolated mammalian photoreceptor precursor cell (PRPCs), isolated mammalian retinal pigment epithelial cells (RPECs), or isolated mammalian corneal endothelial cells (CECs). These ocular cells can be produced by in vitro methods, for example, as described in Zhao, et al., WO 2015/054526, U.S. Pat. No. 10,220,117 B2, and WO 2017/190136 A1, all of which are incorporated by reference in their entirety herein. For example, in certain embodiments, the ocular cells comprise isolated mammalian pRSCs and can be produced and isolated by: (a) culturing isolated pluripotent stem cells (PSCs) (e.g., embryonic stem cells (ESCs) or induced pluripotent stem cells (iPSCs)) from a mammal in a cell culture medium that is free of feeder cells, feeder-conditioned medium or serum so as to produce and grow a culture of the isolated PSCs; and (b) contacting the culture of the isolated PSCs so grown with one or more of an inhibitor for Wnt or TGF-β/BMP signaling so as to differentiate the isolated PSCs of (a) into primitive retinal stem cells thereby producing isolated mammalian pRSCs, as described in Zhao, et al., WO 2015/054526. In certain embodiments, the ocular cells comprise isolated mammalian RGPCs, which can be produced and isolated by: (a) culturing isolated primitive retinal stem cells (pRSCs) from a mammal in a cell culture medium that is free of feeder cells, feeder-conditioned medium or serum so as to produce and grow a culture of the isolated pRSCs; and (b) contacting the culture of the isolated pRSCs so grown with one or more of an inhibitor of Wnt, Notch, or FGFR/VEGFR signaling so as to differentiate the isolated pRSCs into isolated RGPCs, thereby producing isolated mammalian RGPCs, as also described in Zhao, et al., WO 2015/054526.

In addition, in certain embodiments the mammalian retinal progenitor cells and corneal endothelial cells are directly isolated from donor tissues, for example, fetal retina and cornea donor tissues. These ocular cells can be used as a suitable vehicle and durable reservoir to deliver an α-MSH polypeptide to a diseased eye. Like pluripotent stem cells (PSCs) and other tissue stem cells such as mesenchymal stem cells (MSCs), ocular stem/progenitor and precursor cells normally express low levels of immunogenicity markers. In addition, the eye is a well-known immune privileged organ. Recent data from a Phase 2b clinical trial of intravitreal administration of jCells (human fetal retinal progenitor cells) for RP treatment without immunosuppression drug also confirms the safeness of ocular cells for sustainable drug delivery (see, Kuppermann, B., “Intravitreal Injection of Allogeneic Human Retinal Progenitor Cells (Jcell) for Treatment of Retinitis Pigmentosa,” The Retina Society).

In certain aspects, also disclosed are engineered ocular cell lines and related methods of treatment comprising administering such engineered ocular cell lines to a subject. For example, upon the intravitreal administration of cells from such engineered ocular cell lines to a subject, such cells endogenously express a transgene encoding an α-MSH polypeptide and secrete such polypeptide to thereby cause a sustainable ocular cell-mediated intraocular delivery of such polypeptide (e.g., delivery of such polypeptide into a subject's eye in need of treatment of an ocular disease or disorder with such polypeptide).

Gene therapy has been envisioned as a potential “one-and-done” approach to further decrease the treatment burden. The subject's retinal cells can be transduced with adeno-associated virus (AAV) constructs which can produce the therapeutic molecules continuously inside the eye. Thus, it would eliminate the need for repeated injections of an α-MSH polypeptide.

The present inventions disclosed herein, including the ocular cell-mediated sustainable intraocular delivery of an α-MSH polypeptide, provide safe, effective, stable, and long-term release of an α-MSH polypeptide in a subject's eye for treating various eye diseases or disorders. In certain embodiments, the iPSC-derived ocular stem cells (OSCs) are loaded ex vivo with a viral or non-viral an α-MSH polypeptide, such as an α-MSH polypeptide expression cassette as shown in FIG. 2. Once delivered to a subject's eyes, these engineered ocular cells can function as a bio-factory and safe delivery vehicle for the stable and long-term release of an α-MSH polypeptide in the eye.

The ocular cells are generated by either the direct differentiation from source cells or the isolation and expansion of cells from donor tissues. The oscular stem cells (OSCs) have a low immunogenicity profile. After the intravitreal injection, they can form a self-aggregated and free-floating cell mass. In some embodiments, the cells can be injected into the subretinal space, the transplanted cells can integrate and form a layer of cells in the retina. The cell graft can protect and rescue the adjacent retinal neurons via paracrine signaling and/or regenerate retinal neurons. As described herein, the ocular stem cells are desirable carriers. After loaded with an α-MSH polypeptide, such ocular stem cells can deliver an α-MSH polypeptide with retained cell structure and function, such as tissue repair and regeneration with low immunogenicity.

When delivered to a subject's eye(s), the mechanism of action of the engineered ocular cells, which are loaded with an α-MSH polypeptide or an expression cassette, comprises (i) expression and/or secretion of an α-MSH polypeptide in the target area of an eye; (ii) being a stable source of trophic support for dysfunctional ocular tissues; and (iii) being capable of further differentiation to become visual cells of the retina and some of the structural components and connections necessary for sight. Accordingly, in certain embodiments, disclosed herein are methods of treating a subject comprising administering engineered ocular cells to the subject (e.g., administering such engineered ocular cells intraocularly), to thereby cause such cells to express and/or secrete an α-MSH polypeptide in the target area of an eye of the subject, provide trophic support to the dysfunctional ocular tissues of the subject, cause the further differentiation of such cells to visual cells of the retina and some of the structural components and connections necessary for sight. In certain embodiments, these transplanted ocular cells can rescue and replace the damaged cells. With a low immunogenicity profile, these transplanted ocular cells may provide a long-term release of an α-MSH polypeptide, which would eliminate the need for repeated (for example every 6-10 days, or every 4-8 weeks) direct intravitreal rejection of a viral vector carrying an α-MSH polypeptide as that in other delivery methods, which often have off-target and immunogenicity issues.

As the engineered ocular stem cells are prepared in vitro, and ocular stem cells can be made in large quantity with high efficiency and low cost, they can serve as off-the-shelf ocular cells as an abundant source for using as a cell-mediated an α-MSH polypeptide carrier in the disclosed cell therapy approach. Thus, the present invention and the off-the-shelf ocular cell therapy approaches disclosed herein have a significant advantage over the current gene therapy approaches, in that ocular cell therapy could target a large population of patients regardless the underlying disease-causing genetic mutations, while the current gene therapy may only target the patients carrying a specific mutated gene. In addition, the engineered cells can be extensively characterized in vitro for its safety and efficacy prior to clinical development.

The presently described sustained ocular cell-mediated intraocular delivery of cellular therapeutics and the treatment approaches disclosed herein can not only protect and rescue retinal tissue via paracrine signaling and molecular exchanges between the grafted healthy cells and dying host retinal cells, but also are able to integrate and regenerate new RPE and photoreceptor cells. The foregoing represents a significant advantage over the RPE cell transplantation methods currently in development for treating geographic atrophy AMD due to progressive degeneration of RPE cells and photoreceptors. Because the RPE cell transplantation method mainly provides trophic protection for the remaining photoreceptors, no cell regeneration involved, and not suitable for the advanced stage patients, who only has a few residual photoreceptors left in the eye.

In some embodiments, the ocular stem cells transduced with lentiviral or AAV cargo constructs can ensure high and long-last expression of a vascular endothelial growth factor inhibitor for treating Wet AMD and Diabetic Retinopathy. The dose can be pre-determined and can be delivered safely without a surgical procedure for global delivery without local gradient effect. This sustainable ocular cell-mediated intraocular delivery of cellular therapeutic approach has apparent advantages over the direct injection methods for delivering an α-MSH polypeptide, or potentially other biological injectable drugs which requires frequent doses every 4-6 weeks on average. This sustainable approach of the ocular cell-mediated intraocular delivery of an α-MSH polypeptide has apparent advantage over the AAV-based gene therapy, which is serotype with dose limited infection efficiency, and has procedure related adverse events, and immunogenic responses.

Enhancing α-MSH Signaling to Improve iPSC Derived Stem Cell Performance

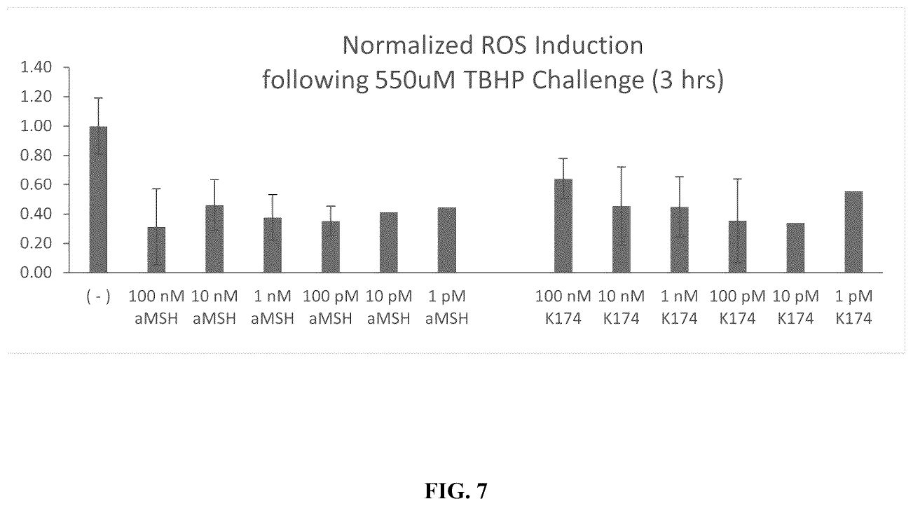

The α-MSH peptide analogues disclosed herein have been shown to confer cytoprotective properties and enhance the survival of ocular stem cells, as shown in FIGS. 7-10. Therefore, in certain embodiments, the present inventions enhance the survival of implanted ocular stem cells (e.g., ABC-101) in vivo. In particular, in certain embodiments, the iPSC-derived ocular stem cells (e.g., ABC-101) may be contacted with one or more α-MSH peptide analogues, thereby rendering such ocular stem cells more resistant to oxidative stress and prolong their survival. In certain embodiments, the iPSC-derived ocular stem cells (e.g., ABC-101) may be formulated or otherwise administered in combination with one or more α-MSH peptide analogues and thereby prolong their survival. In still other embodiments, contacting or combining such iPSC-derived stem cells (e.g., ABC-101) with one or more α-MSH peptide analogues may enhance the neuroprotective effects of such stem cells in retinal neurons. In still other embodiments, contacting or combining such iPSC-derived stem cells (e.g., ABC-101) with one or more α-MSH peptide analogues reduce or otherwise eliminate the need to immunosuppress a patient into which such stem cells are transplanted.

In certain aspects, the α-MSH peptide analogs may protect ocular stem cells (e.g., ABC-101) from ocular environment-related stress. In some aspects, ocular stem cells that are cultured with α-MSH peptide analogs secrete neuroprotective and immune-modulatory factors. In other embodiments, the ocular stem cells contacted with, cultured with or otherwise combined with the α-MSH peptide analogs exhibit upregulated expression of key ocular stem cell transcription factors, as well as melanocortin receptors (MCRs). Notably, such upregulated expression of MCRs is relative, for example, to the between 2- to 67-fold increase in the expression of certain MCR receptors observed by the present inventors in the ocular stem cells disclosed herein, and presented in the table below. Such enhanced melanocortin receptor activity inhibits inflammation and further promotes cell survival or transplanted ocular stem cells.

| Gene | iPSC | ABC-101 | ABC-103 |
|---|---|---|---|
| MC1R | 57.43 | 123.39 | 198.61 |
| MC2R | 6.35 | 54.91 | 55.80 |
| MC3R | 1.46 | 26.78 | 17.58 |
| MC4R | 10.72 | 105.94 | 721.53 |
| MC5R | 9.58 | 6.07 | 16.89 |

### II. Formulation and Delivery

### Pharmaceutical Compositions

According to the present disclosure the compositions (e.g., α-MSH analogs, cells) may be prepared as pharmaceutical compositions. It will be understood that such compositions necessarily comprise one or more active ingredients and, most often, a pharmaceutically acceptable excipient.

Relative amounts of the active ingredient (e.g., α-MSH analogs), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients or subjects.

### Formulations

The compositions of the disclosure can be formulated using one or more excipients. Formulations of the present disclosure can include, without limitation, saline, liposomes, lipid nanoparticles, polymers, peptides, proteins, cells and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

### Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, the formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

In one embodiment, the pharmaceutical compositions comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Acrotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha.-Ionone; Almond Oil; Alpha-Terpineol; Alpha-Tocopherol; Alpha-Tocopherol Acetate, Dl-; Alpha-Tocopherol, Dl-; Aluminum Acetate; Aluminum Chlorohydroxy Allantoinate; Aluminum Hydroxide; Aluminum Hydroxide-Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, Dl-; D & C Red No. 28; D & C Red No. 33; D & C Red No. 36; D & C Red No. 39; D & C Yellow No. 10; Dalfampridine; Daubert 1-5 Pestr (Matte) 1642; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate—Butyl Methacrylate-Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, Dl-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetato Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcellulose; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythritol Ester; Fatty Acids; Fatty Alcohol Citrate; Fatty Alcohols; Fd & C Blue No. 1; Fd & C Green No. 3; Fd & C Red No. 4; Fd & C Red No. 40; Fd & C Yellow No. 10 (Delisted); Fd & C Yellow No. 5; Fd & C Yellow No. 6; Ferric Chloride; Ferric Oxide; Flavor 89-186; Flavor 89-259; Flavor Df-119; Flavor Df-1530; Flavor Enhancer; Flavor Fig 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498 g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O FI-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, Dl-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosyl Hydroxystearate; Hydroxypropyl Cellulose; Hydroxypropyl Methylcellulose 2906; Hydroxypropyl-Beta-cyclodextrin; Hypromellose 2208 (15000 Mpa·S); Hypromellose 2910 (15000 Mpa·S); Hypromellose; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate-Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, Dl-; Lactic Acid, L-; Lactobionic Acid; Lactose; Lactose Monohydrate; Lactose, Hydrous; Laneth; Lanolin; Lanolin Alcohol-Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurtrimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa·S); Methylcelluloses; Methylchloroisothiazolinone; Methylene Blue; Methylisothiazolinone; Methylparaben; Microcrystalline Wax; Mineral Oil; Mono And Diglyceride; Monostearyl Citrate; Monothioglycerol; Multisterol Extract; Myristyl Alcohol; Myristyl Lactate; Myristyl-. Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90 g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy) Propane Anhydride): Sebacic Acid;

Poly(Dimethylsiloxane/Methylvinylsiloxane/Methylhydrogensiloxane) Dimethylvinyl Or Dimethylhydroxy Or Trimethyl Endblocked; Poly(DI-Lactic-Co-Glycolic Acid), (50:50; Poly(DI-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene—Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparaben; Propylparaben; Protamine Sulfate; Protein Hydrolysate; Pvm/Ma Copolymer; Quaternium-15; Quaternium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20 np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecylenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, Dl-; Tenox; Tenox-2; Tert-Butyl Alcohol; Tert-Butyl Hydroperoxide; Tert-Butylhydroquinone; Tetrakis(2-Methoxyisobutylisocyanide) Copper (I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical composition formulations disclosed herein may include cations or anions. As a non-limiting example, formulations may include polymers and complexes with a metal cation.

Formulations of the disclosure may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

### III. Administration and Dosing

### Administration

The compositions of the present disclosure may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the check), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

In some embodiments, compositions may be administered in a way which allows them to cross the blood-brain barrier, vascular barrier, or other epithelial barrier. The compositions of the present disclosure may be administered in any suitable form, either as a liquid solution or suspension, as a solid form suitable for liquid solution or suspension in a liquid solution. The compositions (α-MSH analogs) may be formulated with any appropriate and pharmaceutically acceptable excipient.

In one embodiment, the compositions of the present disclosure may be delivered to a subject via a single route administration.

In one embodiment, the compositions of the present disclosure may be delivered to a subject via a multi-site route of administration. A subject may be administered at 2, 3, 4, 5 or more than 5 sites.

In one embodiment, a subject may be administered the compositions of the present disclosure using a bolus infusion.

In one embodiment, a subject may be administered the compositions of the present disclosure using sustained delivery over a period of minutes, hours or days. The infusion rate may be changed depending on the subject, distribution, formulation or another delivery parameter.

In one embodiment, the compositions of the present disclosure may be delivered by intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In one embodiment, the compositions of the present disclosure may be delivered by oral administration. Non-limiting examples of oral administration include a digestive tract administration and a buccal administration.

In one embodiment, the compositions of the present disclosure may be delivered by intraocular delivery route. A non-limiting example of intraocular administration include an intravitreal injection.

In one embodiment, the compositions of the present disclosure may be delivered by intranasal delivery route. Non-limiting examples of intranasal delivery include administration of nasal drops or nasal sprays.

In some embodiments, the compositions that may be administered to a subject by peripheral injections. Non-limiting examples of peripheral injections include intraperitoneal, intramuscular, intravenous, conjunctival or joint injection.

In one embodiment, the compositions may be delivered by injection into the CSF pathway. Non-limiting examples of delivery to the CSF pathway include intrathecal and intracerebroventricular administration.

In one embodiment, the compositions may be delivered by systemic delivery. As a non-limiting example, the systemic delivery may be by intravascular administration.

In one embodiment, the compositions of the present disclosure may be administered to a subject by intracranial delivery (See, e.g., U.S. Pat. No. 8,119,611; the content of which is incorporated herein by reference in its entirety).

In one embodiment, the compositions of the present disclosure may be administered to a subject by intraparenchymal administration.

In one embodiment, the compositions of the present disclosure may be administered to a subject by intramuscular administration.

In one embodiment, the compositions of the present disclosure are administered to a subject and transduce muscle of a subject. As a non-limiting example, the compositions are administered by intramuscular administration.

In one embodiment, the compositions of the present disclosure may be administered to a subject by intravenous administration.

In one embodiment, the compositions of the present disclosure may be administered to a subject by subcutaneous administration.

In one embodiment, the compositions of the present disclosure may be administered to a subject by topical administration.

In one embodiment, the compositions may be delivered by direct injection into the brain. As a non-limiting example, the brain delivery may be by intrastriatal administration.

In one embodiment, the compositions may be delivered by more than one route of administration. As non-limiting examples of combination administrations, compositions may be delivered by intrathecal and intracerebroventricular, or by intravenous and intraparenchymal administration.

### Parenteral and Injectable Administration

In some embodiments, pharmaceutical compositions, compositions (eg. α-MSH analogs) of the present disclosure may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

In some embodiments, pharmaceutical compositions, (eg. α-MSH analogs) of the present disclosure may be administered rectally and/or vaginally. Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

In some embodiments, pharmaceutical compositions, (eg. α-MSH analogs) of the present disclosure may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, pharmaceutical compositions, (eg. α-MSH analogs) of the present disclosure may be formulated for administration topically. The skin may be an ideal target site for delivery as it is readily accessible. Three routes are commonly considered to deliver pharmaceutical compositions of the present disclosure to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). Pharmaceutical compositions of the present disclosure can be delivered to the skin by several different approaches known in the art.

In some embodiments, the disclosure provides for a variety of dressings (e.g., wound dressings) or bandages (e.g., adhesive bandages) for conveniently and/or effectively carrying out methods of the present disclosure. Typically dressing or bandages may comprise sufficient amounts of pharmaceutical compositions of the present disclosure described herein to allow users to perform multiple treatments.

Dosage forms for topical and/or transdermal administration may include ointments, pastes, creams, lotions, gels, powders, solutions, sprays, inhalants and/or patches. Generally, active ingredients are admixed under sterile conditions with pharmaceutically acceptable excipients and/or any needed preservatives and/or buffers. Additionally, the present disclosure contemplates the use of transdermal patches, which often have the added advantage of providing controlled delivery of pharmaceutical compositions of the present disclosure to the body. Such dosage forms may be prepared, for example, by dissolving and/or dispensing pharmaceutical compositions in the proper medium. Alternatively or additionally, rates may be controlled by either providing rate controlling membranes and/or by dispersing pharmaceutical compositions in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, pharmaceutical compositions of the present disclosure are formulated in depots for extended release. Generally, specific organs or tissues (“target tissues”) are targeted for administration.

In some aspects of the disclosure, pharmaceutical compositions of the present disclosure are spatially retained within or proximal to target tissues. Provided are methods of providing pharmaceutical compositions to target tissues of mammalian subjects by contacting target tissues (which comprise one or more target cells) with pharmaceutical compositions under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions administered to subjects are present intracellularly at a period of time following administration. For example, intramuscular injection to mammalian subjects may be performed using aqueous compositions comprising pharmaceutical compositions of the present disclosure and one or more transfection reagents, and retention is determined by measuring the amount of pharmaceutical compositions present in muscle cells.

Certain aspects of the disclosure are directed to methods of providing pharmaceutical compositions of the present disclosure to a target tissues of mammalian subjects, by contacting target tissues (comprising one or more target cells) with pharmaceutical compositions under conditions such that they are substantially retained in such target tissues. Pharmaceutical compositions comprise enough active ingredient such that the effect of interest is produced in at least one target cell. In some embodiments, pharmaceutical compositions generally comprise one or more cell penetration agents, although “naked” formulations (such as without cell penetration agents or other agents) are also contemplated, with or without pharmaceutically acceptable carriers.

### Pulmonary Administration

In some embodiments, pharmaceutical compositions of the present disclosure may be prepared, packaged, and/or sold in formulations suitable for pulmonary administration. In some embodiments, such administration is via the buccal cavity. In some embodiments, formulations may comprise dry particles comprising active ingredients. In such embodiments, dry particles may have a diameter in the range from about 0.5 nm to about 7 nm or from about 1 nm to about 6 nm. In some embodiments, formulations may be in the form of dry powders for administration using devices comprising dry powder reservoirs to which streams of propellant may be directed to disperse such powder. In some embodiments, self propelling solvent/powder dispensing containers may be used. In such embodiments, active ingredients may be dissolved and/or suspended in low-boiling propellant in sealed containers. Such powders may comprise particles wherein at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally propellants may constitute 50% to 99.9% (w/w) of the composition, and active ingredient may constitute 0.1% to 20% (w/w) of the composition. Propellants may further comprise additional ingredients such as liquid non-ionic and/or solid anionic surfactant and/or solid diluent (which may have particle sizes of the same order as particles comprising active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, comprising active ingredients, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

In some embodiments, pharmaceutical compositions of the present disclosure may be administered nasally and/or intranasally. In some embodiments, formulations described herein useful for pulmonary delivery may also be useful for intranasal delivery. In some embodiments, formulations for intranasal administration comprise a coarse powder comprising the active ingredient and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise powders and/or an aerosolized and/or atomized solutions and/or suspensions comprising active ingredients. Such powdered, aerosolized, and/or aerosolized formulations, when dispersed, may comprise average particle and/or droplet sizes in the range of from about 0.1 nm to about 200 nm, and may further comprise one or more of any additional ingredients described herein.

### Ophthalmic or Otic Administration

In some embodiments, pharmaceutical compositions of the present disclosure may be prepared, packaged, and/or sold in formulations suitable for ophthalmic and/or otic administration. Such formulations may, for example, be in the form of eye and/or car drops including, for example, a 0.1/1.0% (w/w) solution and/or suspension of the active ingredient in aqueous and/or oily liquid excipients. Such drops may further comprise buffering agents, salts, and/or one or more other of any additional ingredients described herein. Other ophthalmically-administrable formulations which are useful include those which comprise active ingredients in microcrystalline form and/or in liposomal preparations. Subretinal inserts may also be used as forms of administration.

### Delivery

### Delivery to Cells

The present disclosure provides a method of delivering to a cell or tissue any of the above-described compositions. The method of delivering the compositions to a cell or tissue can be accomplished in vitro, ex vivo, or in vivo.

### Delivery to Subjects

The present disclosure additionally provides a method of delivering to a subject, including a mammalian subject, any of the above-described compositions comprising administering to the subject said composition, or administering to the subject said formulation.

### Dose and Regimen

The present disclosure provides methods of administering compositions in accordance with the disclosure to a subject in need thereof. The pharmaceutical, diagnostic, or prophylactic compositions of the present disclosure may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific composition employed; the duration of the treatment; drugs used In combination or coincidental with the specific composition employed; and like factors well known in the medical arts.

In certain embodiments, pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations may be converted to vg or viral genomes per kg or into total viral genomes administered by one of skill in the art.

In certain embodiments, pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site.

The desired dosage of the pharmaceutical compositions of the present disclosure may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

The desired dosage of the pharmaceutical compositions of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24 hour period, may be administered by any of these methods, or as a combination of these methods, or by any other methods suitable for a pharmaceutical administration.

In one embodiment, delivery of the pharmaceutical compositions of the present disclosure to a subject provides neutralizing activity to a subject. The neutralizing activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In one embodiment, delivery of the pharmaceutical compositions of the present disclosure results in minimal serious adverse events (SAEs) as a result of the delivery of the pharmaceutical compositions.

### Combinations

The compositions of the present disclosure (eg. α-MSH analogs, cells) may be used in combination with one or more other therapeutic, prophylactic, research or diagnostic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

### IV. Methods and Uses of the Compositions of the Disclosure

The present disclosure provides a method for treating a disease, disorder and/or condition in a mammalian subject, including a human subject, comprising administering to the subject any of the compositions (eg. α-MSH analogs, cells) described herein or administering to the subject any of the described compositions, including pharmaceutical compositions, described herein.

In one embodiment, the compositions of the present disclosure are administered to a subject prophylactically.

In one embodiment, the compositions of the present disclosure are administered to a subject having at least one of the diseases described herein.

In one embodiment, the compositions of the present disclosure are administered to a subject to treat a disease or disorder described herein. The subject may have the disease or disorder or may be at-risk to developing the disease or disorder.

In one embodiment, the compositions of the present disclosure (eg. α-MSH analogs, cells) are part of an active immunization strategy to protect against diseases and disorders. In an active immunization strategy, the compositions are administered to a subject to prevent an infectious disease by activating the subject's production of antibodies that can fight off invading bacteria or viruses.

In one embodiment, the compositions of the present disclosure are part of a passive immunization strategy. In a passive immunization strategy, antibodies against a particular infectious agent are given directly to the subject.

### Diseases

Various autoimmune diseases and autoimmune-related diseases may be treated with pharmaceutical compositions of the present disclosure. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopeniarpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenia purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenia purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA).

Various ocular diseases may be treated with pharmaceutical compositions of the present disclosure. As a non-limiting example, the ocular disease may be thyroid eye disease (TED), Grave' disease (GD) and orbitopathy, Retina Degeneration, Cataract, optic atrophy, macular degeneration, Leber congenital amaurosis, retinal degeneration, cone-rod dystrophy, Usher syndrome, leopard syndrome, photophobia, and photoaversion.

Various kidney diseases may be treated with pharmaceutical compositions of the present disclosure. As a non-limiting example, the kidney disease Abderhalden-Kaufmann-Lignac syndrome (Nephropathic Cystinosis), Abdominal Compartment Syndrome, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Bartter Syndrome, Beeturia, β-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crosser' Nephropathy, Bourbon Virus and Acute Kidney Injury, Burnt Sugarcane Harvesting and Acute Renal Dysfunction, Byetta and Renal Failure, C1q Nephropathy, Cannabinoid Hyperemesis Acute Renal Failure, Cardiorenal syndrome, Carfilzomib-Induced Renal Injury, CFHR5 nephropathy, Charcot-Marie-Tooth Disease with Glomerulopathy, Cherry Concentrate and Acute Kidney Injury, Cholesterol Emboli, Churg-Strauss syndrome, Chyluria, Colistin Nephrotoxicity, Collagen fibrotic Glomerulopathy, Collapsing Glomerulopathy, Collapsing Glomerulopathy Related to CMV, Congenital Nephrotic Syndrome, Conorenal syndrome (Mainzer-Saldino Syndrome or Saldino-Mainzer Disease), Contrast Nephropathy, Copper Sulpfate Intoxication, Cortical Necrosis, Crizotinib-related Acute Kidney Injury, Cryoglobulinemia, Crystalglobulin-Induced Nephropathy, Crystal-Induced Acute Kidney injury, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), Dialysis Disequilibrium Syndrome, Diabetes and Diabetic Kidney Disease, Diabetes Insipidus, Dietary Supplements and Renal Failure, Drugs of Abuse and Kidney Disease, Duplicated Ureter, EAST syndrome, Ebola and the Kidney, Ectopic Kidney, Ectopic Ureter, Edema, Swelling. Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Hair Dye Ingestion and Acute Kidney Injury, Hantavirus Infection Podocytopathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatorenal Syndrome, Herbal Supplements and Kidney Disease, High Blood Pressure and Kidney Disease, HIV-Associated Nephropathy (HIVAN), Horseshoe Kidney (Renal Fusion), Hunne's Ulcer, Hyperaldosteronism, Hypercalcemia, Hyperkalemia, Hypermagnesemia, Hypernatremia, Hyperoxaluria, Hyperphosphatemia, Hypocalcemia, Hypokalemia, Hypokalemia-induced renal dysfunction, Hypokalemic Periodic Paralysis, Hypomagnesemia, Hyponatremia, Hypophosphatemia, IgA Nephropathy, IgG4 Nephropathy, Interstitial Cystitis, Painful Bladder Syndrome (Questionnaire), Interstitial Nephritis, Ivemar's syndrome, Ketamine-Associated Bladder Dysfunction, Kidney Stones, Nephrolithiasis, Kombucha Tea Toxicity, Lead Nephropathy and Lead-Related Nephrotoxicity. Leptospirosis Renal Disease, Light Chain Deposition Disease, Monoclonal Immunoglobulin Deposition Disease, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lyme Disease-Associated Glomerulonephritis, Malarial Nephropathy, Malignancy-Associated Renal Disease, Malignant Hypertension, Malakoplakia, Meatal Stenosis, Medullary Cystic Kidney Disease, Medullary Sponge Kidney, Megaureter, Melamine Toxicity and the Kidney, Membranoproliferative Glomerulonephritis, Membranous Nephropathy. MesoAmerican Nephropathy, Metabolic Acidosis, Metabolic Alkalosis, Methotrexate-related Renal Failure, Microscopic Polyangiitis, Milk-alkalai syndrome, Minimal Change Disease, MDMA (Molly; Ecstacy; 3,4-Methylenedioxymethamphetamine) and Kidney Failure, Multicystic dysplastic kidney, Multiple Myeloma, Myeloproliferative Neoplasms and Glomerulopathy, Nail-patella Syndrome, Nephrocalcinosis, Nephrogenic Systemic Fibrosis, Nephroptosis (Floating Kidney, Renal Ptosis), Nephrotic Syndrome, Neurogenic Bladder, Nodular Glomerulosclerosis, Non-Gonococcal Urethritis, Nutcracker syndrome, Orofaciodigital Syndrome, Orotic Aciduria, Orthostatic Hypotension, Orthostatic Proteinuria, Osmotic Diuresis, Ovarian Hyperstimulation Syndrome, Page Kidney, Papillary Necrosis, Papillorenal Syndrome (Renal-Coloboma Syndrome, Isolated Renal Hypoplasia), Parvovirus B19 and the Kidney, The Peritoneal-Renal Syndrome, Posterior Urethral Valve, Post-infectious Glomerulonephritis, Post-streptococcal Glomerulonephritis, Polyarteritis Nodosa, Polycystic Kidney Disease, Posterior Urethral Valves, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Surgery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia. Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmer Kidney Disease, Sjögre's Syndrome and Renal Disease, Synthetic Cannabinoid Use and Acute Kidney Injury, Kidney Disease Following Hematopoietic Cell Transplantation, Kidney Disease Related to Stem Cell Transplantation, Thin Basement Membrane Disease, Benign Familial Hematuria, Trigonites, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Urethritis Cystica, Ureterocele, Urethral Caruncle, Urethral Stricture, Urinary Incontinence, Urinary Tract Infection, Urinary Tract Obstruction, Vesicointestinal Fistula, Vesicoureteral Reflux, Volatile Anesthetics and Acute Kidney Injury. Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, and Wunderlich syndrome.

Various cardiovascular diseases may be treated with pharmaceutical compositions of the present disclosure. As a non-limiting example, the cardiovascular disease may be Ischemic heart disease also known as coronary artery disease, cerebrovascular disease (Stroke), Peripheral vascular disease, Heart failure, Rheumatic heart disease, and Congenital heart disease.

Various infectious diseases may be treated with pharmaceutical compositions of the present disclosure. As used herein, the term “infectious disease” refers to any disorders caused by organisms such as bacteria, viruses, fungi or parasites. As a non-limiting example, the infectious disease may be Acute bacterial rhinosinusitis, 14-day measles, Acne, Acrodermatitis chronica atrophicans (ACA)-(late skin manifestation of latent Lyme disease), Acute hemorrhagic conjunctivitis, Acute hemorrhagic cystitis, Acute rhinosinusitis, Adult T-cell Leukemia-Lymphoma (ATLL), African Sleeping Sickness, AIDS (Acquired Immunodeficiency Syndrome), Alveolar hydatid, Amebiasis, Amebic meningoencephalitis, Anaplasmosis, Anthrax, Arboviral or parainfectious, Ascariasis (Roundworm infections), Aseptic meningitis, Athlete's foot (Tinea pedis), Australian tick typhus, Avian Influenza, Babesiosis, Bacillary angiomatosis, Bacterial meningitis, Bacterial vaginosis, Balanitis, Balantidiasis, Ban's disease, Barmah Forest virus infection, Bordetellosis (Verruga peruana; Carrio's disease; Oroya fever), Bat Lyssavirus Infection, Bay sore (Chicler's ulcer), Baylisascaris infection (Racoon roundworm infection), Beaver fever, Beef tapeworm, Bejel (endemic syphilis), Biphasic meningoencephalitis, Black Banc, Black death, Black piedra, Blackwater Fever, Blastomycosis, Blennorrhagia of the newborn, Blepharitis, Boils, Bornholm disease (pleurodynia), Borrelia miyamotoi Disease, Botulism, Boutonneuse fever, Brazilian purpuric fever, Break Bone fever, Brill, Bronchiolitis, Bronchitis, Brucellosis (Ban's disease), Bubonic plague, Bullous impetigo, Burkholderia mallei (Glanders), Burkholderia pseudomallei (Melioidosis), Buruli ulcers (also Mycoburuli ulcers), Busse, Busse-Buschke disease (Cryptococcosis), California group encephalitis, Campylobacteriosis, Candidiasis, Canefield fever (Canicola fever; 7-day fever; Wei's disease; leptospirosis; canefield fever), Canicola fever, Capillariasis, Carate, Carbapenem-resistant Enterobacteriaceae (CRE), Carbuncle, Carrio's disease, Cat Scratch fever, Cave disease, Central Asian hemorrhagic fever, Central European tick, Cervical cancer, Chagas disease, Chancroid (Soft chancre), Chicago disease, Chickenpox (Varicella), Chicler's ulcer, Chikungunya fever, Chlamydial infection, Cholera, Chromoblastomycosis, Ciguatera, Clap, Clonorchiasis (Liver fluke infection), Clostridium Difficile Infection, ClostriDium Perfringens (Epsilon Toxin), Coccidioidomycosis fungal infection (Valley fever; desert rheumatism), Coenurosis, Colorado tick fever, Condyloma accuminata, Condyloma accuminata (Warts), Condyloma lata, Congo fever, Congo hemorrhagic fever virus, Conjunctivitis, cowpox, Crabs, Crimean, Croup, Cryptococcosis, Cryptosporidiosis (Crypto), Cutaneous Larval Migrans, Cyclosporiasis, Cystic hydatid, Cysticercosis, Cystitis, Czechoslovak tick, D68 (EV-D68), Dacryocytitis, Dandy fever, Darlin's Disease, Deer fly fever, Dengue fever (1, 2, 3 and 4), Desert rheumatism, Devi's grip, Diphasic milk fever, Diphtheria, Disseminated Intravascular Coagulation, Dog tapeworm, Donovanosis, Donovanosis (Granuloma inguinale), Dracontiasis, Dracunculosis, Duk's disease, Dum Dum Disease, Durand-Nicholas-Favre disease, Dwarf tapeworm, E. coli infection (E. coli), Eastern equine encephalitis, Ebola Hemorrhagic Fever (Ebola virus disease EVD), Ectothrix, Ehrlichiosis (Sennetsu fever), Encephalitis, Endemic Relapsing fever, Endemic syphilis, Endophthalmitis, Endothrix, Enterobiasis (Pinworm infection), Enterotoxin B Poisoning (Staph Food Poisoning), Enterovirus Infection, Epidemic Keratoconjunctivitis, Epidemic Relapsing fever, Epidemic typhus, Epiglottitis, Erysipelis, Erysipeloid (Erysipelothricosis), Erythema chronicum migrans, Erythema infectiosum, Erythema marginatum, Erythema multiforme, Erythema nodosum, Erythema nodosum leprosum, Erythrasma, Espundia, Eumycotic mycetoma, European blastomycosis, Exanthem subitum (Sixth disease), Eyeworm, Far Eastern tick, Fascioliasis, Fievre boutonneuse (Tick typhus), Fifth Disease (erythema infectiosum), Filatow-Duke′ Disease (Scalded Skin Syndrome; Ritte's Disease), Fish tapeworm, Fitz-Hugh-Curtis syndrome (Perihepatitis), Flinders Island Spotted Fever, Flu (Influenza), Folliculitis, Four Corners Disease, Four Corners Disease (Human Pulmonary Syndrome (HPS)), Frambesia, Francis disease, Furunculosis, Gas gangrene, Gastroenteritis, Genital Herpes, Genital Warts, German measles, Gerstmann-Straussler-Scheinker (GSS), Giardiasis, Gilchrist's disease, Gingivitis, Gingivostomatitis, Glanders, Glandular fever (infectious mononucleosis), Gnathostomiasis, Gonococcal Infection (Gonorrhea), Gonorrhea, Granuloma inguinale (Donovanosis), Guinea Worm, Haemophilus Influenza disease, Hamburger disease, Hanse's disease (leprosy), Hantaan disease, Hantaan-Korean hemorrhagic fever, Hantavirus Pulmonary Syndrome, Hantavirus Pulmonary Syndrome (HPS), Hard chancre, Hard measles, Haverhill fever (Rat bite fever), Head and Body Lice, Heartland fever, Helicobacterosis, Hemolytic Uremic Syndrome (HUS), Hepatitis A, Hepatitis B, Hepatitis C, Hepatitis D, Hepatitis E, Herpangina, Herpes-genital, Herpes labialis, Herpes-neonatal, Hidradenitis, Histoplasmosis, Histoplasmosis infection (Histoplasmosis), His-Werner disease, HIV infection, Hookworm infections, Hordeola, Hordcola (Stye), HTLV, HTLV-associated myelopathy (HAM), Human granulocytic ehrlichiosis, Human monocytic ehrlichiosis, Human Papillomavirus (HPV), Human Pulmonary Syndrome, Hydatid cyst, Hydrophobia, Impetigo, Including congenital (German Measles), Inclusion conjunctivitis, Inclusion conjunctivitis (Swimming Pool conjunctivitis-Pannus), Infantile diarrhea, Infectious Mononucleosis, Infectious myocarditis, Infectious pericarditis, Influenza, Isosporiasis, Israeli spotted fever, Japanese Encephalitis, Jock itch, Jorge Lobo disease (lobomycosis), Jungle yellow fever, Junin Argentinian hemorrhagic fever, Kala Azar, Kapos's sarcoma, Keloidal blastomycosis, Keratoconjunctivitis, Kuru, Kyasanur forest disease, LaCrosse encephalitis, Lassa hemorrhagic fever, Legionellosis (Legionnaires Disease), Legionnaire's pneumonia, Lemierr's Syndrome (Postanginal septicemia), Lemming fever, Leprosy, Leptospirosis (Nanukayami fever; Wei's disease), Listeriosis (Listeria), Liver fluke infection, Lob's mycosis, Lockjaw, Loiasis, Louping Ill, Ludwi's angina, Lung fluke infection, Lung fluke infection (Paragonimiasis), Lyme disease, Lymphogranuloma venereum infection (LGV), Machupo Bolivian hemorrhagic fever, Madura foot, Mal del pinto, Malaria, Malignant pustule, Malta fever, Marburg hemorrhagic fever, Masters disease, Maternal Sepsis (Puerperal fever), Measles, Mediterranean spotted fever, Melioidosis (Whitmor's disease), Meningitis, Meningococcal Disease, MERS, Milke's nodule, Molluscum contagiosum, Moniliasis, monkeypox. Mononucleosis, Mononucleosis-like syndrome, Montezum's Revenge, Morbilli, MRSA (methicillin-resistant Staphylococcus aureus) infection, Mucormycosis-Zygomycosis, Multiple Organ Dysfunction Syndrome or MODS, Multiple-system atrophy (MSA), Mumps, Murine typhus, Murray Valley Encephalitis (MVE), Mycoburuli ulcers, Mycoburuli ulcers-Buruli ulcers, Mycotic vulvovaginitis, Myositis, Nanukayami fever, Necrotizing fasciitis, Necrotizing fasciitis-Type 1, Necrotizing fasciitis-Type 2. Negishi, New world spotted fever, Nocardiosis, Nongonococcal urethritis, Non-Polio (Non-Polio Enterovirus), Norovirus infection, North American blastomycosis, North Asian tick typhus, Norwalk virus infection, Norwegian itch, Hara disease, Omsk hemorrhagic fever, Onchocerciasis, Onychomycosis, Opisthorchiasis, Opthalmia neonatorum, Oral hairy leukoplakia, Orf. Oriental Sore, Oriental Spotted Fever, Ornithosis (Parrot fever; Psittacosis), Oroya fever, Otitis externa, Otitis media, Pannus, Paracoccidioidomycosis, Paragonimiasis, Paralytic Shellfish Poisoning (Paralytic Shellfish Poisoning), Paronychia (Whitlow), Parotitis, PCP pneumonia, Pediculosis, Peliosis hepatica, Pelvic Inflammatory Disease, Pertussis (also called Whooping cough), Phaeohyphomycotic, Pharyngoconjunctival fever, Piedra (White Piedra), Piedra (Black Piedra), Pigbel. Pink eye conjunctivitis, Pinta, Pinworm infection, Pitted Keratolysis, Pityriasis versicolor (Tinea versicolor), Plague-Bubonic, Pleurodynia, Pneumococcal Disease, Pneumocystis, Pneumonia, Pneumonic (Plague), Polio or Poliomyelitis, Polycystic hydatid, Pontiac fever, Pork tapeworm, Posada-Wernicke disease, Postanginal septicemia, Powassan, Progressive multifocal leukoencephalopathy, Progressive Rubella Panencephalitis, Prostatitis, Pseudomembranous colitis, Psittacosis, Puerperal fever, Pustular Rash diseases (Small pox), Pyelonephritis, Pylephlebitis, Q-Fever, Quinsy, Quintana fever (5-day fever), Rabbit fever, Rabies, Racoon roundworm infection, Rat bite fever, Rat tapeworm, Reiter Syndrome, Relapsing fever, Respiratory syncytial virus (RSV) infection, Rheumatic fever, Rhod torulosis, Ricin Poisoning, Rickettsialpox, Rickettsiosis, Rift Valley Fever, Ringworm, Ritte's Disease, River Blindness, Rocky Mountain spotted fever, Rose Handle's disease (Sporotrichosis), Rose rash of infants, Roseola, Ross River fever, Rotavirus infection, Roundworm infections, Rubella, Rubeola, Russian spring, Salmonellosis gastroenteritis, San Joaquin Valley fever, Sao Paulo Encephalitis, Sao Paulo fever, SARS, Scabies Infestation (Scabies) (Norwegian itch), Scalded Skin Syndrome, Scarlet fever (Scarlatina), Schistosomiasis, Scombroid, Scrub typhus, Sennetsu fever, Sepsis (Septic shock), Severe Acute Respiratory Syndrome, Severe Acute Respiratory Syndrome (SARS), Shiga Toxigenic Escherichia coli (STEC/VTEC), Shigellosis gastroenteritis (Shigella), Shinbone fever, Shingles, Shipping fever, Siberian tick typhus, Sinusitis, Sixth disease, Slapped check disease, Sleeping sickness, Smallpox (Variola), Snail Fever, Soft chancre, Southern tick associated rash illness, Sparganosis, Spelunker's disease, Sporadic typhus, Sporotrichosis, Spotted fever, Spring, St. Louis encephalitis, Staphylococcal Food Poisoning, Staphylococcal Infection, Strep. throat, Streptococcal Disease, Streptococcal Toxic-Shock Syndrome, Strongyloidiasis, Stye, Subacute Sclerosing Panencephalitis, Subacute Sclerosing Panencephalitis (SSPE), Sudden Acute Respiratory Syndrome, Sudden Rash, Swimmer's ear, Swimmer's Itch, Swimming Pool conjunctivitis, Sylvatic yellow fever, Syphilis, Systemic Inflammatory Response Syndrome (SIRS), Tabes dorsalis (tertiary syphilis), Taeniasis, Taiga encephalitis, Tanne's disease, Tapeworm infections, Temporal lobe encephalitis, Temporal lobe encephalitis, tetani (Lock Jaw), Tetanus Infection, Threadworm infections, Thrush, Tick, Tick typhus, Tinea barbae, Tinea capitis, Tinea corporis, Tinea cruris, Tinea magnum, Tinea nigra, Tinea pedis, Tinea unguium, Tinea versicolor, Torulopsis, Torulosis, Toxic Shock Syndrome, Toxoplasmosis, transmissible spongiform (CJD), Traveler's diarrhea, Trench fever 5, Trichinellids, Trichomoniasis, Trichomycosis axillaris, Trichuriasis, Tropical Spastic Paraparesis (TSP), Trypanosomiasis, Tuberculosis (TB), Tuberculousis, Tularemia, Typhoid Fever, Typhus fever, Ulcus molle, Undulant fever, Urban yellow fever, Urethritis, Vaginitis, Vaginosis, Vancomycin Intermediate (VISA), Vancomycin Resistant (VRSA), Varicella, Venezuelan Equine encephalitis, Verruga peruana, Vibrio cholerae (Cholera), Vibriosis (Vibrio), Vincen's disease or Trench mouth, Viral conjunctivitis, Viral Meningitis, Viral meningoencephalitis, Viral rash, Visceral Larval Migrans, Vomito negro, Vulvovaginitis, Warts, Waterhouse, Wei's disease, West Nile Fever, Western equine encephalitis, Whippl's disease, Whipworm infection, White Piedra, Whitlow, Whitmor's disease, Winter diarrhea, Wolhynia fever, Wool sorter disease, Yaws, Yellow Fever, Yersinosis, Yersinosis (Yersinia), Zahorsk's disease, Zika virus disease, Zoster, Zygomycosis, John Cunningham Virus (JCV), Human immunodeficiency virus (HIV), Influenza virus, Hepatitis B, Hepatitis C, Hepatitis D, Respiratory syncytial virus (RSV), Herpes simplex virus 1 and 2, Human Cytomegalovirus, Epstein-Barr virus, Varicella zoster virus, Coronaviruses, Poxviruses, Enterovirus 71, Rubella virus, Human papilloma virus, Streptococcus pneumoniae, Streptococcus viridans., Staphylococcus aureus (S. aureus), Methicillin-resistant Staphylococcus aureus (MRSA), Vancomycin-intermediate Staphylococcus aureus (VISA), Vancomycin-resistant Staphylococcus aureus (VRSA), Staphylococcus epidermidis (S. epidermidis), Clostridium Tetani, Bordetella pertussis, Bordetella paratussis, Mycobacterium, Francisella Tularensis, Toxoplasma gondii, Candida (C. albicans, C. glabrata, C. parapsilosis, C. tropicalis, C. krusei and C. lusitaniae) and/or any other infectious diseases, disorders or syndromes.

Various toxins may be treated with the pharmaceutical compositions of the present disclosure. Non-limited examples of toxins include Ricin, Bacillus anthracis, Shiga toxin and Shiga-like toxin, Botulinum toxins.

Various tropical diseases may be treated with pharmaceutical compositions of the present disclosure. Non-limited examples of tropical diseases include Chikungunya fever, Dengue fever, Chagas disease, Rabies, Malaria, Ebola virus, Marburg virus, West Nile Virus, Yellow Fever, Japanese encephalitis virus, St. Louis encephalitis virus.

Various foodborne illnesses and gastroenteritis may be treated with pharmaceutical compositions of the present disclosure. Non-limited examples of foodborne illnesses and gastroenteritis include Rotavirus, Norwalk virus (Norovirus), Campylobacter jejuni, Clostridium difficile, Entamoeba histolytica, Helicobacter pyroli, Enterotoxin B of Staphylococcus aureus, Hepatitis A virus (HAV), Hepatitis E, Listeria monocytogenes, Salmonella, Clostridium perfringens, and Salmonella.

Various infectious agents may be treated with pharmaceutical compositions of the present disclosure. Non-limited examples of infectious agents include adenoviruses, Anaplasma phagocytophilum, Ascaris lumbricoides, Bacillus anthracis, Bacillus cereus, Bacteroides sp, Barmah Forest virus, Bartonella bacilliformis, Bartonella henselae, Bartonella quintana, beta-toxin of Clostridium perfringens, Bordetella pertussis, Bordetella parapertussis, Borrelia burgdorferi, Borrelia miyamotoi, Borrelia recurrentis, Borrelia sp., Botulinum toxin, Brucella sp., Burkholderia pseudomallei, California encephalitis virus, Campylobacter, Candida albicans, chikungunya virus, Chlamydia psittaci, Chlamydia trachomatis, Clonorchis sinensis, Clostridium difficile bacteria, Clostridium tetani, Colorado tick fever virus, Corynebacterium diphtheriae, Corynebacterium minutissimum, Coxiella burnetii, coxsackie A, coxsackie B, Crimean-Congo hemorrhagic fever virus, cytomegalovirus, dengue virus, Eastern Equine encephalitis virus, Ebola viruses, echovirus, Ehrlichia chaffeensis., Ehrlichia equi., Ehrlichia sp., Entamoeba histolytica, Enterobacter sp., Enterococcus faecalis, Enterovirus 71, Epstein-Barr virus (EBV), Erysipelothrix rhusiopathiae, Escherichia coli, Flavivirus, Fusobacterium necrophorum, Gardnerella vaginalis, Group B streptococcus, Haemophilus aegyptius, Haemophilus ducreyi, Haemophilus influenzae, hantavirus, Helicobacter pylori, Hepatitis A, Hepatitis B, Hepatitis C. Hepatitis D, Hepatitis E, herpes simplex virus 1 and 2, human herpes virus 6, human herpes Virus 8, human immunodeficiency virus 1 and 2, human T-cell leukemia viruses I and II, influenza viruses (A, B, C), Jamestown Canyon virus, Japanese encephalitis antigenic, Japanese encephalitis virus, John Cunningham virus, juninvirus, Kapos's Sarcoma-associated Herpes Virus (KSHV), Klebsiella granulomatis, Klebsiella sp., Kyasanur Forest Disease virus, La Crosse virus, Lassa virus, Legionella pneumophila, Leptospira interrogans, Listeria monocytogenes, lymphocytic choriomeningitis virus, lyssavirus, Machupovirus, Marburg virus, measles virus, MERS coronavirus (MERS-COV), Micrococcus sedentarius, Mobiluncus sp., Molluscipoxvirus, Moraxella catarrhalis, Morbilli-Rubeola virus, Mumpsvirus, Mycobacterium leprae, Mycobacterium tuberculosis, Mycobacterium ulcerans, Mycoplasma genitalium, Mycoplasma sp, Nairovirus, Neisseria gonorrhoeae, Neisseria meningitidis, Nocardia, Norwalk virus, norovirus, Omsk hemorrhagic fever virus, papilloma virus, parainfluenza viruses 1-3, parapoxvirus, parvovirus B19, Peptostreptococcus sp., Plasmodium sp., polioviruses types I, II, and III, Proteus sp., Pseudomonas aeruginosa, Pseudomonas pseudomallei, Pseudomonas sp., rabies virus, respiratory syncytial virus, ricin toxin, Rickettsia australis, Rickettsia conori, Rickettsia honei, Rickettsia prowazekii, Ross River Virus, rotavirus, rubella virus, Saint Louis encephalitis, Salmonella Typhi, Sarcoptes scabiei, SARS-associated coronavirus (SARS-COV), Serratia sp., Shiga toxin and Shiga-like toxin, Shigella sp., Sin Nombre Virus, Snowshoe hare virus, Staphylococcus aureus, Staphylococcus epidermidis, Streptobacillus moniliformis, Streptococcus pneumoniae, Streptococcus agalactiae, Streptococcus agalactiae, Streptococcus group A-H, Streptococcus pneumoniae, Streptococcus pyogenes, Treponema pallidum subsp. Pallidum, Treponema pallidum var. carateum, Treponema pallidum var. endemicum, Tropheryma whippelii, Ureaplasma urealyticum, Varicella-Zoster virus, variola virus, Vibrio cholerae, West Nile virus, yellow fever virus, Yersinia enterocolitica, Yersinia pestis, and Zika virus.

Various rare diseases may be treated with pharmaceutical compositions of the present disclosure. As used herein, the term “rare disease” refers to any disease that affects a small percentage of the population. As a non-limiting example, the rare disease may be Acrocephalosyndactylies, Acrodermatitis, Addison Disease, Adie Syndrome, Alagille Syndrome, Amylose, Amyotrophic Lateral Sclerosis, Angelman Syndrome, Angiolymphoid Hyperplasia with Eosinophilia, Arnold-Chiari Malformation, Arthritis, Juvenile Rheumatoid, Asperger Syndrome, Bardet-Biedl Syndrome, Barrett Esophagus, Beckwith-Wiedemann Syndrome, Behcet Syndrome, Bloom Syndrome, Bowe's Disease, Brachial Plexus Neuropathies, Brown-Sequard Syndrome, Budd-Chiari Syndrome, Burkitt Lymphoma, Carcinoma 256, Walker, Caroli Disease, Charcot-Marie-Tooth Disease, Chediak-Higashi Syndrome, Chiari-Frommel Syndrome, Chondrodysplasia Punctata, Colonic Pseudo-Obstruction, Colorectal Neoplasms, Hereditary Nonpolyposis, Craniofacial Dysostosis, Creutzfeldt-Jakob Syndrome, Crohn Disease, Cushing Syndrome, Cystic Fibrosis, Dandy-Walker Syndrome, De Lange Syndrome, Dementia, Vascular, Dermatitis Herpetiformis, DiGeorge Syndrome, Diffuse Cerebral Sclerosis of Schilder, Duane Retraction Syndrome, Dupuytren Contracture, Ebstein Anomaly, Eisenmenger Complex, Ellis-Van Creveld Syndrome, Encephalitis, Enchondromatosis, Epidermal Necrolysis, Toxic, Facial Hemiatrophy, Factor XII Deficiency, Fanconi Anemia, Felt's Syndrome, Fibrous Dysplasia, Polyostotic, Fox-Fordyce Disease, Friedreich Ataxia, Fusobacterium, Gardner Syndrome, Gaucher Disease, Gerstmann Syndrome, Giant Lymph Node Hyperplasia, Glycogen Storage Disease Type I, Glycogen Storage Disease Type II, Glycogen Storage Disease Type IV, Glycogen Storage Disease Type V, Glycogen Storage Disease Type VII, Goldenhar Syndrome, Guillain-Barre Syndrome, Hallerman's Syndrome, Hamartoma Syndrome, Multiple, Hartnup Disease, Hepatolenticular Degeneration, Hepatolenticular Degeneration, Hereditary Sensory and Motor Neuropathy, Hirschsprung Disease, Histiocytic Necrotizing Lymphadenitis, Histiocytosis, Langerhans-Cell, Hodgkin Disease, Horner Syndrome, Huntington Disease, Hyperaldosteronism, Hyperhidrosis, Hyperostosis, Diffuse Idiopathic Skeletal, Hypopituitarism, Inappropriate ADH Syndrome, Intestinal Polyps, Isaacs Syndrome, Kartagener Syndrome, Kearns-Sayre Syndrome, Klippel-Feil Syndrome, Klippel-Trenaunay-Weber Syndrome, Kluver-Bucy Syndrome, Korsakoff Syndrome, Lafora Disease, Lambert-Eaton Myasthenic Syndrome, Landau-Kleffner Syndrome, Langer-Giedion Syndrome, Leigh Disease, Lesch-Nyhan Syndrome, Leukodystrophy, Globoid Cell, Li-Fraumeni Syndrome, Long QT Syndrome, Machado-Joseph Disease, Mallory-Weiss Syndrome, Marek Disease, Marfan Syndrome, Meckel Diverticulum, Miege Syndrome, Melkersson-Rosenthal Syndrome, Meniere Disease, Mikulic' Disease, Miller Fisher Syndrome, Mobius Syndrome, Moyamoya Disease, Mucocutaneous Lymph Node Syndrome, Mucopolysaccharidosis I, Mucopolysaccharidosis II, Mucopolysaccharidosis III, Mucopolysaccharidosis IV, Mucopolysaccharidosis VI, Multiple Endocrine Neoplasia Type 1, Munchausen Syndrome by Proxy, Muscular Atrophy, Spinal, Narcolepsy, Neuroaxonal Dystrophies, Neuromyelitis Optica, Neuronal Ceroid-Lipofuscinoses, Niemann-Pick Diseases, Noonan Syndrome, Optic Atrophies, Hereditary, Osteitis Deformans, Osteochondritis, Osteochondrodysplasias, Osteolysis, Essential, Paget Disease Extramammary, Page's Disease, Mammary, Panniculitis, Nodular Nonsuppurative, Papillon-Lefevre Disease, Paralysis, Pelizaeus-Merzbacher Disease, Pemphigus, Benign Familial, Penile Induration, Pericarditis, Constrictive, Peroxisomal Disorders, Peutz-Jeghers Syndrome, Pick Disease of the Brain, Pierre Robin Syndrome, Pigmentation Disorders, Pityriasis Lichenoides, Polycystic Ovary Syndrome, Polyendocrinopathies, Autoimmune, Prader-Willi Syndrome, Pupil Disorders, Rett Syndrome, Reye Syndrome, Rubinstein-Taybi Syndrome, Sandhoff Disease, Sarcoma, Ewin's, Schnitzler Syndrome, Sjogren's Syndrome, Sjogren-Larsson Syndrome, Smith-Lemli-Opitz Syndrome, Spinal Muscular Atrophies of Childhood, Sturge-Weber Syndrome, Sweating, Gustatory, Takayasu Arteritis, Tangier Disease, Tay-Sachs Disease, Thromboangiitis Obliterans, Thyroiditis, Autoimmune, Tietz's Syndrome, Togaviridae Infections, Tolosa-Hunt Syndrome, Tourette Syndrome, Uveomeningoencephalitic Syndrome, Waardenbur's Syndrome, Wegener Granulomatosis, Weil Disease, Werner Syndrome, Williams Syndrome, Wilms Tumor, Wolff-Parkinson-White Syndrome, Wolfram Syndrome, Wolman Disease, Zellweger Syndrome, Zollinger-Ellison Syndrome, and von Willebrand Diseases.

Various neurological diseases may be treated with pharmaceutical compositions of the present disclosure. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adi's Pupil, Adi's Syndrome, Adrenoleukodystrophy, Agenesis of the Corpus Callosum, Agnosia, Aicardi Syndrome, Aicardi-Goutieres Syndrome Disorder, AIDS Neurological Complications, Alexander Disease, Alper' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becke's Myotonia, Behcet's Disease, Bel's Palsy, Benign Essential Blepharospasm, Benign Focal Amyotrophy, Benign Intracranial Hypertension, Bernhardt-Roth Syndrome, Binswange's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushin's Syndrome, Cytomegalic Inclusion Body Disease, Cytomegalovirus Infection, Dancing Eyes-Dancing Feet Syndrome, Dandy-Walker Syndrome, Dawson Disease, De Morsie's Syndrome, Dejerine-Klumpke Palsy, Dementia, Dementia-Multi-Infarct, Dementia-Semantic, Dementia-Subcortical, Dementia With Lewy Bodies, Dentate Cerebellar Ataxia, Dentatorubral Atrophy, Dermatomyositis, Developmental Dyspraxia, Devi's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Er's Palsy, Erb-Duchenne and Dejerine-Klumpke Palsies, Essential Tremor, Extrapontine Myelinolysis, Fabry Disease, Fah's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreic's Ataxia, Frontotemporal Dementia, Gaucher Disease, Generalized Gangliosidoses, Gerstman's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Discase, Hydranencephaly, Hydrocephalus, Hydrocephalus-Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy. Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension. Isaac Syndrome, Joubert Syndrome, Kearns-Sayre Syndrome, Kenned's Disease, Kinsbourne syndrome, Kleine-Levin Syndrome, Klippel-Feil Syndrome, Klippel-Trenaunay Syndrome (KTS), Klüver-Bucy Syndrome, Korsakof's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leig's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehri's Disease, Lupus-Neurological Sequelae, Lyme Disease-Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernous, Niemann-Pick Disease, Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pic's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudo toxoplasmosis syndrome, Pseudotumor Cerebri, Psychogenic Movement, Ramsay Hunt Syndrome I, Ramsay Hunt Syndrome II, Rasmusse's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Rey's Syndrome. Rheumatic Encephalitis, Riley-Day Syndrome, Sacral Nerve Root Cysts, Saint Vitus Dance, Salivary Gland Disease, Sandhoff Disease, Schilde's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomse's Myotonia, Thoracic Outlet Syndrome, Thyrotoxic Myopathy, Tic Douloureux, Tod's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen Disease, Wallenberg's Syndrome, Werdnig-Hoffman Disease, Wernicke-Korsakoff Syndrome, West Syndrome, Whiplash, Whippl's Disease, Williams Syndrome, Wilson Disease, Wolman's Disease, X-Linked Spinal and Bulbar Muscular Atrophy.

Various psychological disorders may be treated with pharmaceutical compositions of the present disclosure. As a non-limiting example, the psychological disorders may be Abulia, Absence epilepsy, Acute stress Disorder, Adjustment Disorders, Adverse effects of medication NOS, Age related cognitive decline, Agoraphobia, Alcohol Addiction, Alzheimer's Disease, Amnesia (also known as Amnestic Disorder), Amphetamine Addiction, Anorexia Nervosa, Anterograde amnesia, Antisocial personality disorder (also known as Sociopathy), Anxiety Disorder (Also known as Generalized Anxiety Disorder), Anxiolytic related disorders, Asperger's Syndrome (now part of Autism Spectrum Disorder), Attention Deficit Disorder (Also known as ADD), Attention Deficit Hyperactivity Disorder (Also known as ADHD), Autism Spectrum Disorder (also known as Autism), Autophagia, Avoidant Personality Disorder, Barbiturate related disorders, Benzodiazepine related disorders, Bereavement, Bibliomania, Binge Eating Disorder, Bipolar disorder (also known as Manic Depression, includes Bipolar I and Bipolar II), Body Dysmorphic Disorder, Borderline intellectual functioning, Borderline Personality Disorder, Breathing-Related Sleep Disorder, Brief Psychotic Disorder, Bruxism, Bulimia Nervosa, Caffeine Addiction, Cannabis Addiction, Catatonic disorder, Catatonic schizophrenia, Childhood amnesia, Childhood Disintegrative Disorder (now part of Autism Spectrum Disorder), Childhood Onset Fluency Disorder (formerly known as Stuttering), Circadian Rhythm Disorders, Claustrophobia, Cocaine related disorders, Communication disorder, Conduct Disorder, Conversion Disorder, Cotard delusion, Cyclothymia (also known as Cyclothymic Disorder), Delerium, Delusional Disorder, dementia, Dependent Personality Disorder (also known as Asthenic Personality Disorder), Depersonalization disorder (now known as Depersonalization/Derealization Disorder), Depression (also known as Major Depressive Disorder), Depressive personality disorder, Derealization disorder (now known as Depersonalization/Derealization Disorder), Dermatillomania, Desynchronosis, Developmental coordination disorder, Diogenes Syndrome, Disorder of written expression, Dyspareunia, Dissocial Personality Disorder, Dissociative Amnesia, Dissociative Fugue, Dissociative Identity Disorder (formerly known as Multiple Personality Disorder), Down syndrome, Dyslexia, Dyspareunia, Dysthymia (now known as Persistent Depressive Disorder), Eating disorder NOS, Ekbom's Syndrome (Delusional Parasitosis), Emotionally unstable personality disorder, Encopresis, Enuresis (bedwetting), Erotomania, Exhibitionistic Disorder, Expressive language disorder, Factitious Disorder, Female Sexual Disorders, Fetishistic Disorder, Folie à deux, Fregoli delusion, Frotteuristic Disorder, Fugue State, Ganser syndrome, Gambling Addiction, Gender Dysphoria (formerly known as Gender Identity Disorder), Generalized Anxiety Disorder, General adaptation syndrome, Grandiose delusions, Hallucinogen Addiction, Haltlose personality disorder, Histrionic Personality Disorder, Primary hypersomnia, Huntington's Disease, Hypoactive sexual desire disorder, Hypochondriasis, Hypomania, Hyperkinetic syndrome, Hypersomnia, Hysteria, Impulse control disorder, Impulse control disorder NOS, Inhalant Addiction, Insomnia, Intellectual Development Disorder, Intermittent Explosive Disorder, Joubert syndrome, Kleptomania, Korsakoff's syndrome, Lacunar amnesia, Language Disorder, Learning Disorders, Major Depression (also known as Major Depressive Disorder), major depressive disorder, Male Sexual Disorders, Malingering, Mathematics disorder, Medication-related disorder, Melancholia, Mental Retardation (now known as Intellectual Development Disorder), Misophonia, Morbid jealousy, Multiple Personality Disorder (now known as Dissociative Identity Disorder), Munchausen Syndrome, Munchausen by Proxy, Narcissistic Personality Disorder, Narcolepsy, Neglect of child, Neurocognitive Disorder (formerly known as Dementia), Neuroleptic-related disorder, Nightmare Disorder, Non Rapid Eye Movement, Obsessive-Compulsive Disorder, Obsessive-Compulsive Personality Disorder (also known as Anankastic Personality Disorder), Oneirophrenia, Onychophagia, Opioid Addiction, Oppositional Defiant Disorder, Orthorexia (ON), Pain disorder, Panic attacks, Panic Disorder, Paranoid Personality Disorder, Parkinson's Disease, Partner relational problem, Passive-aggressive personality disorder, Pathological gambling, Pedophilic Disorder, Perfectionism, Persecutory delusion, Persistent Depressive Disorder (also known as Dysthymia), Personality change due to a general medical condition, Personality disorder, Pervasive developmental disorder (PDD), Phencyclidine related disorder, Phobic disorder, Phonological disorder, Physical abuse, Pica, Polysubstance related disorder, Postpartum Depression, Post-traumatic embitterment disorder (PTED), Post Traumatic Stress Disorder, Premature ejaculation, Premenstrual Dysphoric Disorder, Psychogenic amnesia, Psychological factor affecting medical condition, Psychoneurotic personality disorder, Psychotic disorder, not otherwise specified, Pyromania, Reactive Attachment Disorder, Reading disorder, Recurrent brief depression, Relational disorder, REM Sleep Behavior Disorder, Restless Leg Syndrome, Retrograde amnesia, Retts Disorder (now part of Autism Spectrum Disorder), Rumination syndrome, Sadistic personality disorder, Schizoaffective Disorder, Schizoid Personality Disorder, Schizophrenia, Schizophreniform disorder, Schizotypal Personality Disorder, Seasonal Affective Disorder, Sedative, Hypnotic, or Anxiolytic Addiction, Selective Mutism, Self-defeating personality disorder, Separation Anxiety Disorder, Sexual Disorders Female, Sexual Disorders Male, Sexual Addiction, Sexual Masochism Disorder, Sexual Sadism Disorder, Shared Psychotic Disorder, Sleep Arousal Disorders, Sleep Paralysis, Sleep Terror Disorder (now part of Nightmare Disorder, Social Anxiety Disorder, Somatization Disorder, Specific Phobias, Stendhal syndrome, Stereotypic movement disorder, Stimulant Addiction, Stuttering (now known as Childhood Onset Fluency Disorder), Substance related disorder, Tardive dyskinesia, Tobacco Addiction, Tourettes Syndrome, Transient tic disorder, Transient global amnesia, Transvestic Disorder, Trichotillomania, Undifferentiated Somatoform Disorder, Vaginismus, and Voyeuristic Disorder.

Various lung diseases may be treated with pharmaceutical compositions of the present disclosure. As a non-limiting example, the lung diseases may be Asbestosis, Asthma, Bronchiectasis, Bronchitis, Chronic Cough, Chronic Obstructive Pulmonary Disease (COPD), Croup, Cystic Fibrosis, Hantavirus, Idiopathic Pulmonary Fibrosis, Pertussis, Pleurisy, Pneumonia, Pulmonary Embolism, Pulmonary Hypertension, Sarcoidosis, Sleep Apnea, Spirometry, Sudden Infant Death Syndrome (SIDS), Tuberculosis, Alagille Syndrome, Autoimmune Hepatitis, Biliary Atresia, Cirrhosis, ERCP (Endoscopic Retrograde Cholangiopancreatography), and Hemochromatosis. Nonalcoholic Steatohepatitis, Porphyria, Primary Biliary Cirrhosis, Primary Sclerosing Cholangitis.

Various bone diseases may be treated with pharmaceutical compositions of the present disclosure. As a non-limiting example, the bone diseases may be osteoporosis, neurofibromatosis, osteogenesis imperfecta (OI), rickets, osteosarcoma, achondroplasia, fracture, osteomyelitis, Ewing tumour of bone, osteomalacia, hip dysplasia, Paget disease of bone, marble bone disease, osteochondroma, bone cancer, bone disease, osteochondrosis, osteoma, fibrous dysplasia, cleidocranial dysostosis, osteoclastoma, bone cyst, metabolic bone disease, melorheostosis, callus, Caffey syndrome, and mandibulofacial dysostosis.

Various blood diseases may be treated with pharmaceutical compositions of the present disclosure. As a non-limiting example, the blood diseases may be Anemia and CKD (for health care professionals), Aplastic Anemia and Myelodysplastic Syndromes, Deep Vein Thrombosis, Hemochromatosis, Hemophilia, Henoch-Schönlein Purpura, Idiopathic Thrombocytopenia Purpura, Iron-Deficiency Anemia, Pernicious Anemia, Pulmonary Embolism, Sickle Cell Anemia, Sickle Cell Trait and Other Hemoglobinopathies, Thalassemia, Thrombotic Thrombocytopenia Purpura, and Von Willebrand Disease.

Various diseases associated with TNF-alpha may be treated with the pharmaceutical compositions of the present disclosure. As a non-limiting example, the disease may be respiratory disorder; asthma; allergic and nonallergic asthma; asthma due to infection; asthma due to infection with respiratory syncytial virus (RSV); chronic obstructive pulmonary disease (COPD); a condition involving airway inflammation; eosinophilia; fibrosis and excess mucus production; cystic fibrosis; pulmonary fibrosis; an atopic disorder; atopic dermatitis; urticaria; eczema; allergic rhinitis; allergic enterogastritis; an inflammatory and/or autoimmune condition of the skin; an inflammatory and/or autoimmune condition of gastrointestinal organs; inflammatory bowel diseases (IBD); ulcerative colitis; Crohn's disease; an inflammatory and/or autoimmune condition of the liver; liver cirrhosis; liver fibrosis; liver fibrosis caused by hepatitis B and/or C virus; scleroderma; tumors or cancers; hepatocellular carcinoma; glioblastoma; lymphoma; Hodgkin's lymphoma; a viral infection; a bacterial infection; a parasitic infection; HTLV-1 infection; suppression of expression of protective type 1 immune responses, and suppression of expression of a protective type 1 immune response during vaccination, rheumatoid arthritis, osteoarthritis, juvenile chronic arthritis, septic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegene's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmid's syndrome, adult (acute) respiratory distress syndrome, alopecia, alopecia greata, seronegative arthropathy, arthropathy, Reite's disease, psoriatic arthropathy, ulcerative colitic arthropathy, enteropathic synovitis, chlamydia, yersinia and salmonella associated arthropathy, spondyloarthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, hepatitis B, hepatitis C, common varied immunodeficiency (common variable hypogammaglobulinemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögre's disease associated lung disease, ankylosing spondylitis associated lung disease, vasculitic diffuse lung disease, hemosiderosis associated lung disease, drug-induced interstitial lung disease, fibrosis, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopoenia, autoimmune neutropenia, renal disease NOS, glomerulonephritides, microscopic vasculitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjörgren's syndrome, Takayas's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxedema, photogenic uveitis, primary vasculitis, vitiligo acute liver disease, chronic liver diseases, alcoholic cirrhosis, alcohol-induced liver injury, cholestasis, idiosyncratic liver disease, drug-Induced hepatitis, non-alcoholic steatohepatitis, allergy and asthma, group B streptococci (GBS) infection, mental disorders (e.g., depression and schizophrenia), Th2 Type and Th1 Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma) abetalipoproteinemia, acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti-CD3 therapy, antiphospholipid syndrome, anti-receptor hypersensitivity reactions, aortic and peripheral aneurysms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chronic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, corypalmine, coronary artery disease, Creutzfeldt-Jakob disease, culture negative sepsis, cystic fibrosis, cytokine therapy associated disorders, dementia pugilistica, demyelinating diseases, dengue hemorrhagic fever, dermatitis, dermatologic conditions, diabetes, diabetes mellitus, diabetic arteriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein-Barr virus infection, erythromelalgia, extrapyramidal and cerebellar disorders, familial hemophagocytic lymphohistiocytosis, fetal thymus implant rejection, Friedreic's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallervorden-Spatz disease, Hashimoto's thyroiditis, hay fever, heart transplant rejection, hemochromatosis, hemodialysis, hemolytic uremic syndrome/thrombolytic thrombocytopenia purpura, hemorrhage, hepatitis (A), His bundle arrhythmias, HIV infection/HIV neuropathy, Hodgkin's disease, hyperkinetic movement disorders, hypersensitivity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, asthenia, infantile spinal muscular atrophy, inflammation of the aorta, influenza a, ionizing radiation exposure, iridocyclitis/uveitis/optic neuritis, ischemia-reperfusion injury, ischemic stroke, juvenile rheumatoid arthritis (JRA), juvenile spinal muscular atrophy, Kapos's sarcoma, kidney transplant rejection, legionella, leishmaniasis, leprosy, lesions of the corticospinal system, lipedema, liver transplant rejection, lymphedema, malaria, malignant lymphoma, malignant histiocytosis, malignant melanoma, meningitis, meningococcemia, metabolic/idiopathic, migraine headache, mitochondrial multi-system disorder, mixed connective tissue disease, monoclonal gammopathy, multiple myeloma, multiple systems degenerations (Menzel, Dejerine-Thomas, Shy-Drager, and Machado-Joseph), myasthenia gravis, Mycobacterium avium intracellulare, Mycobacterium tuberculosis, myelodysplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-Hodgkins lymphoma, occlusion of the abdominal aorta and its branches, occlusive arterial disorders, OKT3® therapy, orchitis/epididymitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherosclerotic disease, peripheral vascular disorders, peritonitis, pernicious anemia, Pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, progressive supranuclear palsy, primary pulmonary hypertension, radiation therapy, Raynaud's phenomenon and disease, Raynaud's disease, Refsum's disease, regular narrow QRS tachycardia, renovascular hypertension, reperfusion injury, restrictive cardiomyopathy, sarcomas, scleroderma, senile chorea, senile dementia of Lewy body type, seronegative arthropathies, shock, sickle cell anemia, skin allograft rejection, skin changes syndrome, small bowel transplant rejection, solid tumors, specific arrhythmias, spinal ataxia, spinocerebellar degenerations, streptococcal myositis, structural lesions of the cerebellum, subacute sclerosing panencephalitis, syncope, syphilis of the cardiovascular system, systemic anaphylaxis, systemic inflammatory response syndrome, systemic onset juvenile rheumatoid arthritis, T-cell or FAB ALL, telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, viral encephalitis/aseptic meningitis, viral-associated hemophagocytic syndrome, Wernicke-Korsakoff syndrome, Wilson's disease, xenograft rejection of any organ or tissue, acute coronary syndromes, acute idiopathic polyneuritis, acute inflammatory demyelinating polyradiculoneuropathy, acute ischemia, adult Still's disease, alopecia greata, anaphylaxis, anti-phospholipid antibody syndrome, aplastic anemia, arteriosclerosis, atopic eczema, atopic dermatitis, autoimmune dermatitis, autoimmune disorder associated with streptococcus infection, autoimmune enteropathy, autoimmune hearing loss, autoimmune lymphoproliferative syndrome (ALPS), autoimmune myocarditis, autoimmune premature ovarian failure, blepharitis, bronchiectasis, bullous pemphigoid, cardiovascular disease, catastrophic antiphospholipid syndrome, celiac disease, cervical spondylosis, chronic ischemia, cicatricial pemphigoid, clinically isolated syndrome (CIS) with risk for multiple sclerosis, conjunctivitis, childhood onset psychiatric disorder, chronic obstructive pulmonary disease (COPD), dacryocystitis, dermatomyositis, diabetic retinopathy, diabetes mellitus, disk herniation, disk prolapse, drug induced immune hemolytic anemia, endocarditis, endometriosis, endophthalmitis, episcleritis, erythema multiforme, erythema multiforme major, gestational pemphigoid, Guillain-Barré syndrome (GBS), hay fever, Hughes syndrome, idiopathic Parkinson's disease, idiopathic interstitial pneumonia, IgE-mediated allergy, immune hemolytic anemia, inclusion body myositis, infectious ocular inflammatory disease, inflammatory demyelinating disease, inflammatory heart disease, inflammatory kidney disease, IPF/UIP, iritis, keratitis, keratojunctivitis sicca, Kussmaul disease or Kussmaul-Meier disease, Landry's paralysis, Langerhan's cell histiocytosis, livedo reticularis, macular degeneration, microscopic polyangiitis, morbus Bechterew, motor neuron disorders, mucous membrane pemphigoid, multiple organ failure, myasthenia gravis, myelodysplastic syndrome, myocarditis, nerve root disorders, neuropathy, non-A non-B hepatitis, optic neuritis, osteolysis, ovarian cancer, pauciarticular JRA, peripheral artery occlusive disease (PAOD), peripheral vascular disease (PVD), peripheral artery disease (PAD), phlebitis, polyarteritis nodosa (or periarteritis nodosa), polychondritis, polymyalgia rheumatica, poliosis, polyarticular JRA, polyendocrine deficiency syndrome, polymyositis, polymyalgia rheumatica (PMR), post-pump syndrome, primary Parkinsonism, prostate and rectal cancer and hematopoietic malignancies (leukemia and lymphoma), prostatitis, pure red cell aplasia, primary adrenal insufficiency, recurrent neuromyelitis optica, restenosis, rheumatic heart disease, sapho (synovitis, acne, pustulosis, hyperostosis, and osteitis), scleroderma, secondary amyloidosis, shock lung, scleritis, sciatica, secondary adrenal insufficiency, silicone associated connective tissue disease, Sneddon-Wilkinson dermatosis, spondylitis ankylosans, Stevens-Johnson syndrome (SJS), systemic inflammatory response syndrome, temporal arteritis, toxoplasmic retinitis, toxic epidermal necrolysis, transverse myelitis, TRAPS (tumor necrosis factor receptor associated periodic syndrome), type 1 allergic reaction, type II diabetes, urticaria, usual interstitial pneumonia (UIP), vasculitis, vernal conjunctivitis, viral retinitis, Vogt-Koyanagi-Harada syndrome (VKH syndrome), wet macular degeneration, wound healing, yersinia or salmonella associated arthropathy.

Various receptor for advanced glycation endproducts (RAGE) diseases may be treated with the pharmaceutical compositions of the present disclosure. As a non-limiting example, the disease may be Amyotrophic Lateral Sclerosis, Brachial Plexus Injury, Brain Injury, including traumatic brain injury, Cerebral Palsy, Frideric's Ataxia, Guillain Barre, Leukodystrophies, Multiple Sclerosis, Post Polio, Spina Bifida, Spinal Cord Injury, Spinal Muscle Atrophy, Spinal Tumors, Stroke, Transverse Myelitis, dementia, senile dementia, mild cognitive impairment, Alzheimer-related dementia, Huntington's chorea, tardive dyskinesia, hyperkinesias, manias, Morbus Parkinson, steel-Richard syndrome, Dow's syndrome, myasthenia gravis, nerve trauma, vascular amyloidosis, cerebral hemorrhage I with amyloidosis, brain inflammation, Frideric's ataxia, acute confusion disorder, amyotrophic lateral sclerosis, glaucoma, Alzheimer's disease, diabetic nephropathy, sepsis, rheumatoid arthritis and related inflammatory diseases.

Various neurite degenerative diseases may be treated with the pharmaceutical compositions of the present disclosure. As a non-limiting example, the disease may be multiple sclerosis, Parkinson's disease, Alzheimer's disease, Tay-Sachs disease, Niemann-Pick disease, Gauche's disease, Hurle's syndrome, Huntington's disease, amyotrophic lateral sclerosis, idiopathic inflammatory demyelinating diseases, vitamin B12 deficiency, central pontine myelinolysis, tabes dorsalis, transverse myelitis, Devi's disease, progressive multifocal leukoencephalopathy, optic neuritis, traumatic injury to the CNS, an ischemic cerebral stroke, glaucoma, diabetic retinopathy, age-dependent macular degeneration, and a leukodystrophy.

Various neurological diseases may be treated with the pharmaceutical compositions of the present disclosure. As a non-limiting example, the disease may be Amyotrophic Lateral Sclerosis, Brachial Plexus Injury, Brain Injury, including traumatic brain injury, Cerebral Palsy, Guillain Barre, Leukodystrophies, Multiple Sclerosis, Post Polio, Spina Bifida, Spinal Cord Injury, Spinal Muscle Atrophy, Spinal Tumors, Stroke, Transverse Myelitis, dementia, senile dementia, mild cognitive impairment, Alzheimer-related dementia, Huntington's chorea, tardive dyskinesia, hyperkinesias, manias, Morbus Parkinson, steel-Richard syndrome, Dow's syndrome, myasthenia gravis, nerve trauma, vascular amyloidosis, cerebral hemorrhage I with amyloidosis, brain inflammation, acute confusion disorder, amyotrophic lateral sclerosis, glaucoma and Alzheimer's disease.

Various cancers may be treated with pharmaceutical compositions of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

Types of carcinomas which may be treated with the compositions of the present disclosure include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma.

Types of sarcomas which may be treated with the compositions of the present disclosure include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kapos's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Aski's tumor, Ewin's sarcoma (primitive neuroectodermal tumor), malignant hemangioendothelioma, malignant schwannoma, osteosarcoma, and chondrosarcoma.

As a non-limiting example, the cancer which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Page's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma, Testicular cancer, Throat cancer, Thymoma/thymic carcinoma, Thyroid cancer, Tongue cancer, Tonsil cancer, Transitional cell cancer. Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

### Research Applications

The compositions of the present disclosure may also be used as research tools. The compositions of the disclosure may be used as in any research experiment, e.g., in vivo or in vitro experiments. In some embodiments, the compositions of the disclosure may be used in cultured cells. The cultured cells may be derived from any origin known to one with skill in the art, and may be as non-limiting examples, derived from a stable cell line, an animal model or a human patient or control subject. In a non-limiting example, the compositions of the disclosure may be used in in vivo experiments in animal models (i.e., mouse, rat, rabbit, dog, cat, non-human primate, guinea pig, ferret, c-elegans, drosophila, zebrafish, or any other animal used for research purposes, known in the art). In another non-limiting example, the compositions of the disclosure may be used in human research experiments or human clinical trials.

### Combination Applications

The compositions of the disclosure may be used as a combination therapy with any other therapeutic molecule known in the art. The therapeutic molecule may be approved by the US Food and Drug Administration or may be in clinical trial or at the preclinical research stage. The therapeutic molecule may utilize any therapeutic modality known in the art.

In some embodiments, melanocortin receptor agonist (e.g., α-MSH peptide analogs) of the present disclosure are administered to the subject in combination with one or more stem cell therapies. In one embodiment, the melanocortin receptor agonist (e.g., α-MSH peptide analogs) are combined with cells from the one or more stem cell therapies ex vivo, prior to administration of the one or more stem cell therapies to the subject. In some embodiments, the melanocortin receptor agonist (e.g., α-MSH peptide analogs) of the present disclosure are administered to the subject in combination with one or more retinal stem cell therapies. Examples of retinal stem cells and retinal stem cell therapies which can be used in treatments of the present disclosure include those described in U.S. Pat. No. 10,220,117, the content of which is incorporated herein by reference in its entirety, as related to methods of producing, isolating, preparing, engineering, and using (e.g., therapeutic use with melanocortin receptor agonists) mammalian retinal stem cells. In some embodiments, the melanocortin receptor agonist (e.g., α-MSH peptide analogs) of the present disclosure are administered to the subject in combination with one or more corneal stem cell therapies (e.g., therapies using corneal endothelial cells). Examples of corneal stem cells and corneal stem cell therapies which can be used in treatments of the present disclosure include those described in US20190119633, the content of which is incorporated herein by reference in its entirety, as related to methods of producing, isolating, preparing, engineering, and using (e.g., therapeutic use with melanocortin receptor agonists) mammalian corneal stem cells, such as human corneal endothelial cells. In some embodiments, the co-administration of the melanocortin receptor agonist (e.g., α-MSH peptide analogs) with the one or more stem cell therapies results in enhanced neuroprotection for the cells. In some embodiments, the co-administration of the melanocortin receptor agonist (e.g., α-MSH peptide analogs) with the one or more stem cell therapies (e.g., one or more retinal stem cell therapies) results in long-term inner retinal survival and integration upon administration. In some embodiments, the co-administration of the melanocortin receptor agonist (e.g., α-MSH peptide analogs) with the one or more stem cell therapies (e.g., one or more retinal stem cell therapies) results in axonal regeneration upon administration.

### Therapeutic Applications

The present disclosure additionally provides a method for treating any diseases and/or disorders in a mammalian subject, including a human subject, comprising administering to the subject any of the compositions or pharmaceutical compositions of the disclosure. In some embodiments, diseases and/or disorders treated according to the methods described herein include, but are not limited to, immune system and autoimmune diseases, inflammatory diseases, ocular diseases, Parkinson's Disease (PD), Dementia with Lewy Bodies (DLB), Multiple System Atrophy (MSA), decreased muscle mass, Spinal muscular atrophy (SMA) Alzheimer's disease (AD), Amyotrophic lateral sclerosis (ALS), Huntington's Disease (HD), Multiple sclerosis (MS), stroke, migraine, pain, neuropathies, psychiatric disorders including schizophrenia, bipolar disorder, autism, cancer, and systemic diseases of the blood, heart and bone.

The present disclosure provides a method for administering to a subject in need thereof, including a human subject, a therapeutically effective amount of compositions of the disclosure to slow, stop or reverse disease progression. As a non-limiting example, disease progression may be measured by tests or diagnostic tool(s) known to those skilled in the art. As another non-limiting example, disease progression may be measured by change in the pathological features of the brain, CSF or other tissues of the subject.

### Immune System & Autoimmune Disease

Human immune system is a complex mechanism for identifying and removing harmful environmental agents and repairing the harm and damage caused by them. The basis of the immune system is ability to identify body's own substances from substances acquired. The immune response system can be divided into innate and adaptive systems. The innate system is present at all times and includes macrophages, dendritic cells, myeloid cells (neutrophils, mast cells, basophils, eosinophils) NK cells, complement factors and cytokines. The adaptive system responses to infectious agents, and include T and B lymphocytes, antibodies and cytokines. Activation of T and B cells in the absence of an infectious agents leads to autoimmune diseases (see, e.g., Mackay et al., 2001, N Engl J Med, Vol. 345, No. 5, and references therein). Autoimmune diseases may affect a number of body's tissues and functions, e.g., joints, skin, blood vessels, muscles, organs, intestine etc. Autoimmune diseases arise from and overactive and misguided immune response to body's natural tissues and species. Autoimmune diseases and conditions include, but are not limited to, rheumatoid arthritis, diabetes type 1, systemic lupus erythematosus, celiac sprue, psoriasis, Graves' disease, and Lyme disease. Autoimmune diseases may be caused by infections, drugs, environmental irritants, toxins, and/or genetic factors. Autoimmune diseases affect up to 50 million individuals in the US. Two most common autoimmune diseases are rheumatoid arthritis and autoimmune thyroiditis, together affecting approximately 5% of population in Western countries.

Though medical therapies for autoimmune diseases exits, the diseases may still significantly lower the quality of life, or even be fatal. There remains a need for medical therapies affecting the pathophysiology of autoimmune diseases. Autoimmune disease pathophysiology is associated with a number of factors and may be prevented and/or treated by antibodies targeting associated proteins. Such targets include, but are not limited to, infectious agents; environmental triggers (e.g. gliadin); targets affecting cytokinome production or signalling (e.g. TNFa (tumor necrosis factor alpha), IL-1 (interleukin 1-receptor), IL-2 (interleukin-2), IL-2R (interleukin-2 receptor), IL-7 (interleukin-7), IL-10 (interleukin-10), IL-10R (interleukin-10 receptor), interferon-y, STAT-3 (Signal transducer and activator of transcription 3), STAT-4 (Signal transducer and activator of transcription 4), TGF beta (transforming growth factor beta), T cell trans TGF beta); T cell regulators (e.g. CTLA4 (Cytotoxic T-Lymphocyte-Associated Protein 4)); complement components (e.g. C1 and C4); TNFa (tumor necrosis factor alpha) and TNFb (tumor necrosis factor beta); T cell regulators (e.g. CD1); epitopes of B and T cells; and/or other targets, such as those associated with B and C cells. (see, e.g. Mackay et al., 2001, N Engl J Med, Vol. 345, No. 5, and references therein).

In some embodiments, methods of the present disclosure may be used to treat subjects suffering from an autoimmune disease. In some cases, methods of the present disclosure may be used to treat subjects suspected of developing an autoimmune disease. Compositions and methods of using the compositions described in the present disclosure may be used to prevent, manage and/or treat immune system and autoimmune disease.

In some embodiments, methods of the present disclosure may be used to reduce immune response. In some embodiments, methods of the present disclosure may be used to reduce immune response in a subject.

### Inflammatory Disorders and Inflammation

Inflammation is a natural response of the body to an irritation e.g. by infection, damaged cells or other harmful agents. The purpose of the inflammation is to remove the cause of irritation and necrotic cells and damaged tissues and initiate cell and tissue repair. Inflammation has a role in majority of diseases. Inflammatory disorders are abnormalities in the body's ability to regulate inflammation. Over 100 disorders associated with high level of inflammation have been identified, including, but not limited to, Alzheimer's, ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease, and ulcerative colitis. Many inflammatory disorders are severe, and even life-threatening. Antibodies targeting proteins associated with inflammation may be used to prevent, manage or treat inflammatory disorders as well as inflammation associated diseases.

A large number of proteins are associated in inflammation, including, but not limited to, TNF (anti-tumor necrosis factor), IL-1R (Interleukin-1 receptor), IL-6R (Interleukin-6 receptor), Alpha integrin subunit, CTLA4 (Cytotoxic T-Lymphocyte-Associated Protein 4), and CD20 (see, e.g., Kotsovilis and Andreakos, 2014, Michael Steinitz (ed.), Human Monoclonal Antibodies: Methods and Protocols, Methods in Molecular Biology, vol. 1060, and references therein). For example, adalimumab (developed by Abbot Laboratories) is a TNF-targeting antibody for rheumatoid arthritis and other arthritides, psoriasis, and Crohn's disease and Natalizumab (developed by Biogen Idec) is an antibody targeting alpha 4-integrin for treatment of Crohn's disease. Additionally, plethora of cytokines, chemokines, adhesion and co-stimulatory molecules, receptors, as well as diverse cell types, may have a role in inflammatory diseases.

In some embodiments, methods of the present disclosure may be used to treat subjects suffering from an inflammatory disease. In some cases, methods of the present disclosure may be used to treat subjects suspected of developing an inflammatory disease. Compositions and methods of using the compositions described in the present disclosure may be used to prevent, manage and/or treat inflammatory disorders and inflammation.

In some embodiments, methods of the present disclosure may be used to reduce inflammation. In some embodiments, methods of the present disclosure may be used to reduce inflammation in a subject.

### Graft Versus Host Disease

Graft versus host disease (GVHD) occurs when the donor's T cells (the graft) view the patient's healthy cells (the host) as foreign, and attack and damage them. There are two main categories of GVHD: acute graft-versus-host disease and chronic graft-versus-host disease. Each type affects different organs and tissues and has different signs and symptoms. Acute GVHD can affect the skin, the gastrointestinal tract or the liver whereas chronic GVHD may involve a single organ or several organs In some embodiments, methods of the present disclosure may be used to treat subjects suffering from graft versus host disease. In some cases, methods of the present disclosure may be used to treat subjects suspected of developing graft versus host disease.

### Ocular Diseases

Eye is an organ comprising a number of components, including the cornea, aqueous humor, lens, vitreous humor, retina, the retinal pigment epithelium, and choroid. Ocular diseases are conditions affecting the different tissues of the eye. A number of diseases and disorders affect the different components of the eye, and may cause impaired vision, full or partial blindness, irritation, dryness, sensitivity, photophobia, and/or light aversion.

Complement in the eye has an important role in protecting the eye from infections and in modulation of the immune and inflammatory responses. In normal eye, the complement activity is at low level and is regulated by membrane bound and soluble intraocular complement regulatory proteins. Disturbance of the balance between complement activation and complement inhibition may lead to damage to self-tissue (see, e.g., Jha et al., 2007, Mol Immunol; 44(16): 3901-3908, and references therein). The complement system may be activated in three pathways. The classical pathway is activated by immune complexes or substances and involves e.g. complement components C1, C2, C3, C4, C3a, C5, C5a, C5b, C6, C7, C8, C9 and C5b-9. The alternative pathway activates complement component C3 when in interaction with e.g. zymosan, or lipopolysaccharide surfaces, additionally involving, e.g. Factor B, Factor Ba, Factor Bb, Factor D, and Factor P. The third activation pathway is the lectin pathway, and is related to interaction of certain serum lectins, e.g. mannose binding lectin (MBL), mannose and N-acetyl glucosamine residues present in bacterial cell walls. Complement activation is associated with a number of ocular diseases, such as, but not related, age-related macular degeneration (AMD), diabetic retinopathy, choroidal neovascularization (CNV), uveitis, diabetic macular edema, pathological myopia, von Hippel-Lindau disease, histoplasmosis of the eye, Central Retinal Vein Occlusion (CRVO), corneal neovascularization, and retinal neovascularization, choroidal neovascularization, and other ocular conditions involving complement activation. Antibodies targeting the associated complement components may be used to diagnose, manage and/or treat such ocular diseases.

Age-related macular degeneration (AMD) is a major cause of irreversible loss of central vision in the elderly world wide. AMD leads to gradually worsening vision. AMD does not result in blindness, but may affect daily life. Wet AMD is caused by abnormal blood vessels behind the retina grow under the macula and leak blood and fluid that damage the macula. Wet AMD may be treated with laser coagulation and medication to reverse or stop the growth of blood vessels. Dry AMD is caused by break down of the light sensitive cells in the macula. As of today there is no treatment for dry AMD.

There remains a need for prevention, management and treatment therapies for wet and dry AMD. AMD is associated with complement components, as described above. In addition, AMD is associated with proteins such as, but not limited to, VEGF (Vascular endothelial growth factor), EPO (Erythropoietin), EPOR (EPO receptor), Interleukins IL-1β, IL-17A, Il-10, TNFα (tumor necrosis factor alpha), or FGFR2 (Fibroblast Growth Factor Receptor). Antibodies targeting the AMD associated complement and growth proteins may be used to treat AMD. For example, bevacizumab and ranibizumab (developed by Genentech Inc.) are antibodies targeting VEGF-A to slow down growth of new blood vessels.

Corneal diseases affect the cornea and the conjunctiva. Cornea and conjunctiva form the outer surface of the eye, which is exposed to external environment, and are susceptible to infection agents, trauma, and/or exposure to chemicals, toxins, allergens etc. Cornea is also affected by autoimmune conditions, nutritional deficiencies and cancer. Corneal diseases may cause e.g. loss of vision, blurred vision, tearing, light sensitivity and pain. Diseases affecting cornea include, but are not limited to, keratitis, corneal dystrophy, corneal degeneration, Fuchs' dystrophy, cancer of cornea, and keratoconjunctivitis. Though surgical and medical treatment therapies for corneal diseases exist, in some cases, the diseases still remain severe and may cause blindness. There remains a need to efficient therapies for prevention, management and treatment of corneal diseases. Complement components of the cornea and the conjunctiva present in a normal eye include, but are not limited to, C1, C2, C3, C4, C5, C6, C7, Factor P (properdin) and factor B. Complement may have a role in corneal diseases, and antibodies targeting complement components of the eye may be used for prevention, treatment and/or management of corneal diseases.

Uveitis is an inflammation of the uvea, comprising the iris, choroids, and ciliary body. Early symptoms include eye redness, pain, irritation and blurred vision. Uveitis may lead to transient or permanent loss of vision. Uveitis may be associated with other diseases and conditions, such as infections, systemic diseases, non-infectious and autoimmune diseases. Complement components associated with an autoimmune form of uveitis include C3b and C4b. Uveitis may be managed or treated with vitrectomy, immunosuppressive drugs, corticosteroids or cytotoxic medication. However, despite the existing therapies, autoimmune uveitis is a serious condition and may lead to full or partial blindness. There remains a need for therapies for prevention, management, and treatment of uveitis targeting pathophysiology of the disease.

Retinopathy is a disease resulting from neovascularization (excessive growth of blood vessels) in the light-sensitive tissue of the eye, retina. Retinopathy may result in impaired vision or partial or full blindness. Retinopathy may be caused by systemic diseases, e.g., diabetes, or hypertension, trauma, excessive sun light exposure or ionizing radiation. Retinopathy is often treated with laser therapy. Medical treatments, such as antibodies, to control the growth of blood vessels, are also applied. However, despite the existing treatment methods, retinopathy is still a severe condition and may lead to blindness. Diabetic retinopathy is one of the leading causes of vision loss in middle-aged individuals. There remains a need for new therapies for prevention, management and/or treatment of retinopathy. For example, antibodies targeting blood vessel growth (e.g., vascular endothelial growth factor (VEGF), complement components (e.g., C3, C4, C1q, C9, C4b), and cluster of differentiation proteins (e.g., CD55, CD59) may be used for prevention, management and/or treatment of different retinopathies.

Photophobia is a condition referring to abnormal sensitivity or aversion to light. Photophobia is related to a number of ocular and nervous system diseases and disorders. Photophobia may be caused by damage to cornea or retina, albinism, overstimulation of the photoreceptors, excessive electric pulses to the central nervous system, or optic nerve. Photophobia may be associated with migraine, nervous system disorders (e.g., autism, dyslexia, encephalitis), infections (e.g., rabies, Lyme disease, mononucleosis), eye disorders (e.g. uveitis, corneal diseases, retinal diseases, scarring or trauma to cornea). In some embodiments, methods of the present disclosure may be used to treat subjects suffering from ocular diseases. In some cases, methods of the present disclosure may be used to treat subjects suspected of developing ocular diseases.

### Systemic Diseases of the Blood, Heart and Bone

Systemic diseases are a category of conditions affecting the whole body, or many tissues and organs of the body. Systemic conditions associated with the blood, blood vessels, and heart, include, but are not limited to, heart failure, acute coronary syndrome, atherosclerosis, hypertension, lung disease, cardiomyopathy, hyperlipidemia, hypercholesterolemia, hypertriglyceridemia, blood clotting, cardiopulmonary bypass, myocardial infection, platelet aggregation and hemolytic diseases. In general, such conditions affect individual's quality of life and may be life-threatening. Cardiovascular diseases, referring to heart and blood vessels related conditions, are the leading cause of death worldwide. There remains a need for therapies affecting the pathophysiology of systemic heart, blood and blood circulation diseases. Antibodies for treating such conditions have been developed, targeting proteins such as, but not limited to, selectin P, integrin αIIbβ3, GPIIb/IIIa, RHD (Rh blood group, D antigen), PCSK9 (proprotein convertase subtilisin/kexin type 9), oxLDL (Oxidized low-density lipoprotein), CD20 (B-lymphocyte antigen), ANGPTL3 (Angiopoietin-Like 3), F9 (human factor 9), F10 (human factor 10), TFPI (Tissue Factor Pathway Inhibitor (Lipoprotein-Associated Coagulation Inhibitor)), CD41 (Integrin, Alpha 2b (Platelet Glycoprotein lib Of lib/IIIa Complex, Antigen CD41)).

In some embodiment, methods of the present disclosure may be used to treat subjects suffering from blood, blood circulation and heart related systemic diseases. In some cases, methods of the present disclosure may be used to treat subjects suspected of developing systemic blood, blood circulation and heart related systemic diseases.

Osteoporosis is a disease characterized by a reduced bone mineral density, and disrupted bone microarchitecture. Individuals with osteoporosis have a high susceptibility to bone fractures. Osteoporosis causes disability especially in the elderly, and may be fatal.

There are medical therapies for management of the osteoporosis, and other conditions associated with reduced bone density, such as calcitonin, bisphosphonates, estrogen replacement and selective estrogen modulators for prevention of bone loss, and anabolic agents to increase bone mass and bone mineral density. However, the present medical therapies have side effects and/or require frequent administration. There remains a need for efficient and long lasting medical therapy affecting the pathophysiology of osteoporosis and other conditions associated with reduced bone density, such as antibody therapies. Antibodies for treatment of osteoporosis are on the market, e.g., blosozumab (developed by Eli Lilly and Co.) targeting sclerostin (SOST) for increasing bone density, and denosumab (developed by Amgen) targeting TNFSF11 (Tumor Necrosis Factor (Ligand) Superfamily, Member 11) for treatment of bone loss.

In some embodiments, methods of the present disclosure may be used to treat subjects suffering from osteoporosis and/or other conditions associated with reduced bone density. In some cases, methods of the present disclosure may be used to treat subjects suspected of developing osteoporosis and/or other conditions associated with reduced bone density. In some embodiments, compositions and methods of using the compositions described in the present disclosure may be used to prevent, manage and/or treat systemic diseases of the blood, heart and/or bone.

### V. Kits and Devices

In one embodiment, the disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the compositions (eg. α-MSH analogs, cells) of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means.

In some embodiments, kits may include instructions for employing kit components as well the use of any other reagent not included in the kit. Instructions may include variations that may be implemented.

In some embodiments, the compositions (eg. α-MSH analogs, cells) of the present disclosure may delivered to a subject using a device known in the art for suitable purposes.

### VII. Definitions

At various places in the present specification, substituents of compounds of the present disclosure are disclosed in groups or in ranges. It is specifically intended that the present disclosure include each and every individual subcombination of the members of such groups and ranges.

About: As used herein, the term “about” means+/−10% of the recited value.

Activation: As used herein, the term “activation” includes a response in cells when exposed or treated with a stimulus. Activation may refer to differentiation, maturation, and/or polarization. The stimuli that trigger activation may be both chemical and physical, or a combination thereof. Physical stimuli can include the compression of cells in a matrix or the stretching of cells on a surface, which activate mechanosensitive ion channels and adhesion-sensitive integrins (a special class of transmembrane receptors). Chemical stimuli may include for e.g., free-floating molecules, membrane-bound signals from other cells, and chemical cues from the matrix the cells are growing in or on.

Activity: As used herein, the term “activity” refers to the condition in which things are happening or being done. Compositions of the disclosure may have activity and this activity may involve one or more biological events.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached.” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active.

Cell therapy: As used herein, the term “cell therapy” refers to the transfer of cellular material derived from a source to a subject in need thereof for the treatment, prevention, cure, diagnosis, or mitigation of diseases or injuries. The source may be autologous, allogeneic, or xenogeneic.

Cell therapy product: As used herein, a “cell therapy product” refers to the cells or population of cells that have been propagated, expanded, selected, pharmacologically treated, genetically engineered or otherwise altered ex vivo in their biological characteristics, to be administered to a subject in need thereof.

Chimeric antigen receptor (CAR): As used herein, the term “chimeric antigen receptor” or “CAR” refers to an artificial chimeric protein comprising at least one extracellular portion that binds or interacts with a specific protein or an antigen), a transmembrane domain and an intracellular signaling domain. The extracellular portion of the CAR may be derived from an antibody or a fragment thereof. The extracellular portion of the CAR may optionally have a spacer domain and/or a co-stimulatory domain. A CAR may also engineered used to generate a cytotoxic cell carrying the CAR.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Modified: As used herein “modified” refers to a changed state or structure of a molecule of the disclosure. Molecules may be modified in many ways including chemically, structurally, and functionally.

Naturally Occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Operably linked: As used herein, the phrase “operably linked” refers to a functional connection between two or more molecules, constructs, transcripts, entities, moieties or the like.

Patient: As used herein, “patient” refers to a subject who may seek or be in need of treatment, requires treatment, is receiving treatment, will receive treatment, or a subject who is under care by a trained professional for a particular disease or condition.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

The details of one or more embodiments of the disclosure are set forth in the accompanying description below. Although any materials and methods similar or equivalent to those described herein can be used in the practice or testing of the present disclosure, the preferred materials and methods are now described. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples.

### VIII. ENUMERATED EMBODIMENTS

Embodiment 1. A cell therapy method comprising, a) obtaining a population of cells from an organism or a cell source, b) contacting the population of cells in vitro or ex vivo with one or more α-MSH peptide analogs of SEQ ID NO: 1-79, and c) transplanting the population of cells into a subject in need thereof.

Embodiment 2. The method of Embodiment 1, further comprising expanding the population of cells prior to contacting the population of cells with the one or more α-MSH peptide analogs of SEQ ID NO: 1-79.

Embodiment 3. The method of Embodiment 1, further comprising expanding the population of cells after contacting the population of cells with the one or more α-MSH peptide analogs of SEQ ID NO: 1-79.

Embodiment 4. The method of Embodiment 1, wherein the population of cells is treated with one or more α-MSH peptide analogs for at least 24 hours.

Embodiment 5. The method of Embodiment 1, wherein the population of cells comprises one or more cell types selected from group consisting of a stem cell, a progenitor cell, and a somatic cell.

Embodiment 6. The method of Embodiment 5, wherein the population of cells is autologous, allogeneic, or xenogeneic.

Embodiment 7. The method of Embodiment 5 or Embodiment 6, wherein the population of cells comprises a stem cell, wherein the stem cell is an embryonic stem cell, an adult stem cell or an induced pluripotent stem cell.

Embodiment 8. The method of Embodiment 5 or Embodiment 6, wherein the population of cells comprises a progenitor cell, wherein the progenitor cell is a neural progenitor cell, a liver progenitor cell, corneal epithelial progenitor cells, a photoreceptor progenitor cell, a monoblast cell, myeloblast cell, or a hematopoietic progenitor cell.

Embodiment 9. The method of Embodiment 5 or Embodiment 6, wherein the population of cells comprises a somatic cell, wherein the somatic cell is an immune cell, a fibroblast, a chondrocyte, a keratinocyte, a hepatocyte, or a pancreatic cell.

Embodiment 10. The method of Embodiment 1, wherein the population of cells is engineered to express at least one payload.

Embodiment 11. The method of Embodiment 9, wherein the at least one payload of interest is one or more of a protein of interest, a fusion polypeptide, an antibody, an antigen, a chimeric antigen receptor (CAR), a T cell receptor (TCR), a safety switch, and/or a regulatory switch.

Embodiment 12. The method of Embodiment 1-9 wherein the cells are genetically modified using a gene editing system of a CRISPR, a TALEN, a Zn-Finger, and a vector delivery systems.

Embodiment 13. The method of Embodiment 8, wherein the gene editing system is delivered to a cell via a vector delivery system.

Embodiment 14. The method of Embodiment 9, wherein the vector delivery system is a RNA, DNA, or viral vector delivery system.

Embodiment 15. The method of any one of Embodiments 1-14, wherein the population of cells are activated prior to transplanting the population of cells.

Embodiment 16. The method of any one of Embodiments 1-15, wherein the population of cells expresses MCRs.

Embodiment 17. A cell therapy product produced by the method of any one of Embodiments 1-16.

### IX. EXAMPLES

### Example 1. General Synthesis of Polypeptides

Polypeptides of the present disclosure can be synthesized by one skilled in the art using any known methods in the art. The polypeptides were synthesized by solid phase chemical peptide synthesis (SPPS) methods. The polypeptides were constructed from their individual amino acids. The amino acids can be covalently bonded to one another through functional groups, as is known in the art, where such functional groups may be present on the amino acids or introduced onto the components using one or more steps. When necessary and/or desired, certain moieties on the amino acids may be protected using blocking groups, as is known in the art, see, e.g., Green & Wuts, Protective Groups in Organic Synthesis (John Wiley & Sons) (1991).

For example, the polypeptides were synthesized using standard solid-phase Fmoc methods. The N-terminus was protected with the Fmoc group, which is stable in acid, but removable by base. Any side chain functional groups were protected with base stable, acid labile groups. The synthesis was typically performed on a peptide synthesizer using standard protocols with Rink amide resin. This resin is insoluble in the solvents used for synthesis, making it relatively simple and fast to wash away excess reagents and by-products. All amino acids were obtained from commercial sources unless otherwise noted. Coupling reagents known in the art can be used. Generally, the coupling reagent was 2-(6-chloro-1-H-benzotriazole-1yl)-1,1,3,3,-tetramethylaminium hexafluorophosphate (HCTU) and the base is diisopropylethylamine (DIEA). Peptides were generally cleaved from resin with trifluoroacetic acid (TFA) and water. The crude peptides were then purified on HPLC. Fractions containing the pure peptide were collected and lyophilized and all peptides were analyzed by LC-MS.

The linear peptides were made in a high-throughput, combinatorial fashion, e.g., using a high-throughput multi-channel combinatorial synthesizer. The peptide cyclization step was done on resin or in solution phase based on the sequences.

The peptides were further modified with, for example, N-terminal blocking agents. The N-terminus can be capped with addition of phenylbutyric acid (PBA), 3,5-dihydroxyphenylacetic acid (HyPA), (3-methoxyphenyl) acetic acid (MoPA), 3-hydroxy-5-methoxybenzeneacetic acid (HymBA), 4-pyridylacetyl acid (PyAA) etc. Such blocking agents can improve binding and confer increased corneal permeability to the peptide.

### Example 2. Co-Administration of α-MSH Analogs and Ocular Stem Cells

iPSC derived ocular stem cells (OSCs) were cultured with an analog of α-MSH peptide. The iPSC derived OSCs have a lower immunogenic profile exhibited by reduced expression of HLA genes and co-stimulatory molecules CD40 and CD86. In addition, effective inhibitors of immune response (e.g., CD47, FAS, and TGFB2) are significantly elevated, as are certain members of the MCR family, which may decrease immune response and increase implant viability.

Co-culture of one or more α-MSH peptide analogs with the OSCs is expected to result in prolonged survival of the cells, as well as enhanced neuroprotective effects in retinal neurons. In some aspects, it is expected that a subject or patient will not need to be immunosuppressed prior to administering the α-MSH peptide analogs in combination with the OSCs. The α-MSH peptide analogs may protect cells from ocular environment-related stress. In some aspects, OSCs cultured with α-MSH peptide analogs secreted neuroprotective and immune-modulatory factors. In addition, the OSCs exhibited upregulated expression of key ocular stem cell transcription factors, as well as MCRs.

A pharmaceutical composition comprising OSCs and at least one analog of α-MSH peptide will be administered to the eye of a subject. The pharmaceutical composition may be administered via IVT injection or via intraretinal deposit. The secretion of neurotrophic factors will be assessed and measured, e.g., to determine if the factors reach the retina (e.g., reaches the photoreceptors and/or retinal ganglion cells).

### Example 3. Constructs of α-MSH Peptide Expression Cassette In Vitro

### Construction of α-MSH Peptide Expression Vector:

The expression of an α-MSH peptide coding sequence may be driven by human eukaryotic translation elongation factor 1 alpha1 short form promoter (EFS) in a mammalian gene expression lentiviral vector. The vector also carries a mCherry marker expression cassette under the control of the CMV promoter.

### Cell Culture, Virus Packaging, and Transduction:

For the lentivirus packaging, the construct is co-transfected with the envelope plasmid encoding VSV-G and packaging plasmids encoding Gag/Pol and Rev into HEK293T packaging cells. After 48 hours of incubation, the supernatant is collected and centrifuged to remove cell debris and then filtered. Lentiviral particles are subsequently concentrated with PEG. The virus titer is determined by the p24 ELISA method. Then the recombinant lentivirus is used to transduce human ocular progenitor cells in a culture dish. The transfected cells can be visualized by the mCherry marker expression. The mCherry positive cells could be further enriched by fluorescence-activated cell sorting (FACS).

### Expression and Secretion of α-MSH Peptide In Vitro:

The consumed medium of lentiviral transfected ocular progenitor cells is collected. The concentration of released α-MSH protein in the conditioned medium is measured by ELISA using known concentrations of α-MSH peptide to generate a standard curve.

### Example 4. Intraocular Delivery of α-MSH Peptide

The lentiviral transfected ocular progenitor cells loaded with α-MSH peptide can then be used to injected into the vitreous (1), the anterior chamber (2), the subretinal space (3), or the suprachoroidal space (4) (FIG. 2) of a patient's eye in need of treatment with the composition having therapeutic effects, such as α-MSH peptide to treat neovascular ocular conditions. The lentiviral transfected ocular progenitor cells loaded with α-MSH peptide can continuously produce and release α-MSH peptide into the surround ocular tissues. Such sustained release of the α-MSH peptide with very low immunogenicity can eliminate the need for frequent intravitreal injection currently being used in other methods, increase patient compliance, and thus provide much higher treatment effects in treating ocular diseases or disorders.

### Example 5. In Vivo Topical Administration Study-Rabbit

To evaluate the pharmacokinetics of the α-MSH peptide formulations following topical administration a study in rabbits was undertaken. Animals were acclimated to the study and, upon completion of the acclimation period, each animal was physically examined for determination of suitability for study participation. Animals determined to be in good health were randomized into study groups (N=51; 17 animals per study group and 3 animals per control group). Animals were uniquely identified by corresponding cage card number and ear tag.

The K173 (KR-072) peptide was formulated in a 5 mg/ml sterile solution with HPMC, histidine buffer, and mannitol. The designated peptide formulations (35 μL) were administered to the eye of each animal using a calibrated pipette. With the animal manually restrained, the upper eyelid of the eye was gently elevated to expose the cornea. Treatment was then applied to the cornea without contacting the eye with the pipette tip. The animal was then allowed to blink several times while still manually restrained to distribute the applied solution over the eye prior to being returned to the cage. Treatment was administered once daily (QD) or twice daily (BID) with approximately 8 hours between BID doses. Group 1 was administered a single dose, Group 2 received 4 doses, Group 3 received 8 doses. Samples were collected from 3 animals/group 30 min, 1 hr, 2 hr, 4 hr, and 8 hr post-last dose and from 2 animals/group 24 hours post-last dose.

A veterinary ophthalmologist performed complete ocular examinations using a slit lamp biomicroscope and indirect ophthalmoscope to evaluate ocular surface morphology and anterior and posterior segment inflammation on all animals prior to injection to serve as a baseline for enrollment into the study as well as on specific study days. Animals were not tranquilized for the examinations.

Following euthanasia, eyes were enucleated, rinsed 2× with fresh ice-cold PBS, and dabbed dry with gauze. Aqueous humor was removed via a 27- or 30-gauge syringe, placed into a pre-weighted 2 mL polypropylene tube, reweighed, and snap frozen by immersion in liquid nitrogen. The cornea was harvested fresh from each eye, placed into a pre-weighted re-enforced 2 mL polypropylene tube, re-weighed and snap frozen in liquid nitrogen. The remaining ocular tissues were discarded. Ocular samples were stored −80° C. All samples were placed into individual vials and weighed. Aqueous humor and cornea samples were collected.

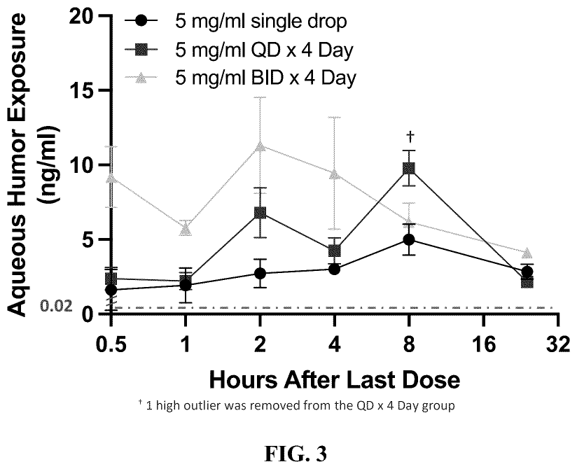

As shown in FIG. 3, K173 (KR-072) was shown to be delivered to the target tissue. K173 was shown to have prolonged ocular surface retention and Tₘₐₓ after a single dose. Aqueous humor (AH) concentrations were shown to increase with repeated dosing. In fact, measurable levels were detected after 24 hours in all dose groups. The concentrations of K173 in the aqueous humor was measured for each group at different time points and the values are provided in Table 6 below.

TABLE 6
Concentration of K173 (KR-072) in Rabbit Aqueous Humor

| | K173 | K173 | | K173 | K173 |
|---|---|---|---|---|---|
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 30 Min | Rabbit Aqueous | Rabbit Aqueous | 30 Min | Rabbit Aqueous | Rabbit Aqueous |
| Animal | Humor | Humor | Animal | Humor | Humor |
| ID | OS Sample | OD Sample | ID | OS Sample | OD Sample |
| 11407 | BQL (1.00) | 8.44 | 11538 | 3.87 | 1.90 |
| 11408 | 1.36 | BQL(1.00) | 11539 | 1.30 | BQL(1.00) |
| 11409 | BQL(1.00) | BQL(1.00) | 11540 | 5.23 | 1.90 |
| Mean | 1.36 | 8.44 | Mean | 3.47 | 1.90 |
| SD | NA | NA | SD | 1.995 | 0.00375 |
| % CV | NA | NA | % CV | 57.5% | 0.2% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 1 hr | Rabbit Aqueous | Rabbit Aqueous | 1 hr | Rabbit Aqueous | Rabbit Aqueous |
| Animal | Humor | Humor | Animal | Humor | Humor |
| ID | OS Sample | OD Sample | ID | OS Sample | OD Sample |
| 11410 | BQL(1.00) | 1.13 | 11541 | BQL(1.00) | 1.60 |
| 11525 | 7.15 | BQL(1.00) | 11542 | 3.37 | 3.74 |
| 11526 | 3.30 | BQL(1.00) | 11543 | 1.62 | 2.90 |
| Mean | 5.22 | 1.13 | Mean | 2.49 | 2.75 |
| SD | 2.73 | NA | SD | 1.24 | 1.07 |
| % CV | 52.2% | NA | % CV | 49.8% | 39.1% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 2 hr | Rabbit Aqueous | Rabbit Aqueous | 2 hr | Rabbit Aqueous | Rabbit Aqueous |
| Animal | Humor | Humor | Animal | Humor | Humor |
| ID | OS Sample | OD Sample | ID | OS Sample | OD Sample |
| 11527 | 2.90 | 7.40 | 11544 | 11.0 | 9.23 |
| 11528 | 1.46 | 1.38 | 11545 | 10.7 | 5.64 |
| 11529 | 1.30 | 1.95 | 11546 | 1.80 | 2.40 |
| Mean | 1.89 | 3.58 | Mean | 7.84 | 5.76 |
| SD | 0.882 | 3.33 | SD | 5.23 | 3.41 |
| % CV | 46.7% | 93.0% | % CV | 66.7% | 59.3% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 4 hr | Rabbit Aqueous | Rabbit Aqueous | 4 hr | Rabbit Aqueous | Rabbit Aqueous |
| Animal | Humor | Humor | Animal | Humor | Humor |
| ID | OS Sample | OD Sample | ID | OS Sample | OD Sample |
| 11530 | 3.56 | 2.94 | 11547 | 6.69 | 4.61 |
| 11531 | 4.04 | 2.79 | 11548 | 2.48 | 6.84 |
| 11532 | 2.46 | 2.31 | 11549 | 2.41 | 2.39 |
| Mean | 3.35 | 2.68 | Mean | 3.86 | 4.61 |
| SD | 0.811 | 0.329 | SD | 2.45 | 2.23 |
| % CV | 24.2% | 12.3% | % CV | 63.5% | 48.3% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 8 hr | Rabbit Aqueous | Rabbit Aqueous | 8 hr | Rabbit Aqueous | Rabbit Aqueous |
| Animal | Humor | Humor | Animal | Humor | Humor |
| ID | OS Sample | OD Sample | ID | OS Sample | OD Sample |
| 11404 | 3.34 | 3.90 | 11535 | 6.41 | 11.3 |
| 11405 | 5.09 | 3.40 | 11536 | 9.88 | 8.07 |
| 11406 | 4.17 | 10.0 | 11537 | 28.1 | 13.2 |
| Mean | 4.20 | 5.77 | Mean | 14.8 | 10.9 |
| SD | 0.875 | 3.68 | SD | 11.6 | 2.62 |
| % CV | 20.8% | 63.8% | % CV | 78.7% | 24.1% |
| | K173 | K173 | | K173 | K173 |
| Group 1 | Concentrations | Concentrations | Group 2 | Concentrations | Concentrations |
| Timepoint | (ng/mL) in | (ng/mL) in | Timepoint | (ng/mL) in | (ng/mL) in |
| 24 hr | Rabbit Aqueous | Rabbit Aqueous | 24 hr | Rabbit Aqueous | Rabbit Aqueous |
| Animal | Humor | Humor | Animal | Humor | Humor |
| ID | OS Sample | OD Sample | ID | OS Sample | OD Sample |
| 11533 | 2.34 | 4.32 | 11575 | 2.30 | 2.34 |
| 11534 | 2.58 | 2.11 | 11576 | 2.36 | 1.61 |
| Mean | 2.46 | 3.22 | Mean | 2.33 | 1.98 |
| SD | 0.175 | 1.56 | SD | 0.0431 | 0.518 |
| % CV | 7.1% | 48.4% | % CV | 1.8% | 26.2% |
| | K173 | K173 | | K173 | K173 |
| Group 3 | Concentrations | Concentrations | Group 3 | Concentrations | Concentrations |
| Timepoint | (ng/mL) in | (ng/mL) in | Timepoint | (ng/mL) in | (ng/mL) in |
| 30 Min | Rabbit Aqueous | Rabbit Aqueous | 1 hr | Rabbit Aqueous | Rabbit Aqueous |
| Animal | Humor | Humor | Animal | Humor | Humor |
| ID | OS Sample | OD Sample | ID | OS Sample | OD Sample |
| 11582 | 8.54 | 9.70 | 11585 | 7.48 | 5.13 |
| 11583 | 12.7 | 16.5 | 11586 | 6.40 | 6.00 |
| 11584 | 3.10 | 4.63 | 11587 | 5.80 | 3.86 |
| Mean | 8.11 | 10.3 | Mean | 6.56 | 5.00 |
| SD | 4.80 | 5.94 | SD | 0.855 | 1.08 |
| % CV | 59.3% | 57.8% | % CV | 13.0% | 21.5% |
| | K173 | K173 | | K173 | K173 |
| Group 3 | Concentrations | Concentrations | Group 3 | Concentrations | Concentrations |
| Timepoint | (ng/mL) in | (ng/mL) in | Timepoint | (ng/mL) in | (ng/mL) in |
| 2 hr | Rabbit Aqueous | Rabbit Aqueous | 4 hr | Rabbit Aqueous | Rabbit Aqueous |
| Animal | Humor | Humor | Animal | Humor | Humor |
| ID | OS Sample | OD Sample | ID | OS Sample | OD Sample |
| 11588 | 10.8 | 9.68 | 11591 | 6.53 | 4.11 |
| 11589 | 22.5 | 18.5 | 11592 | 2.82 | 3.82 |
| 11590 | 3.11 | 3.28 | 11593 | 12.6 | 26.8 |
| Mean | 12.1 | 10.5 | Mean | 7.33 | 11.6 |
| SD | 9.76 | 7.62 | SD | 4.96 | 13.2 |
| % CV | 80.5% | 72.8% | % CV | 67.7% | 113.9% |
| | K173 | K173 | | K173 | K173 |
| Group 3 | Concentrations | Concentrations | Group 3 | Concentrations | Concentrations |
| Timepoint | (ng/mL) in | (ng/mL) in | Timepoint | (ng/mL) in | (ng/mL) in |
| 8 hr | Rabbit Aqueous | Rabbit Aqueous | 24 hr | Rabbit Aqueous | Rabbit Aqueous |
| Animal | Humor | Humor | Animal | Humor | Humor |
| ID | OS Sample | OD Sample | ID | OS Sample | OD Sample |
| 11578 | 9.10 | 3.65 | 11594 | 3.94 | 3.34 |
| 11580 | 4.24 | 3.33 | 11595 | 4.96 | 4.24 |
| 11581 | 5.93 | 10.8 | Mean | 4.45 | 3.79 |
| Mean | 6.42 | 5.92 | SD | 0.717 | 0.639 |
| SD | 2.46 | 4.21 | % CV | 16.1% | 16.9% |
| % CV | 38.4% | 71.2% | | | |

*BQL (Conc.) = Below Quantitation Limit (ng/mL); NA: Not Applicable

### Example 6. In Vivo Topical Administration Study-Minipig

To evaluate the effects of the α-MSH peptide analog formulations following topical administration a study in minipigs was undertaken. Animals were acclimated to the study environment for a minimum of 1 week. At the completion of the acclimation period, each animal was physically examined for determination of suitability for study participation. Examinations included, but were not limited to, the skin and external ears, eyes, and abdomen, neurological behavior, and general body condition. Animals determined to be in good health were randomized into one of three study groups (N=12; 4 animals per study group) and uniquely identified by corresponding cage card number and ear tag.

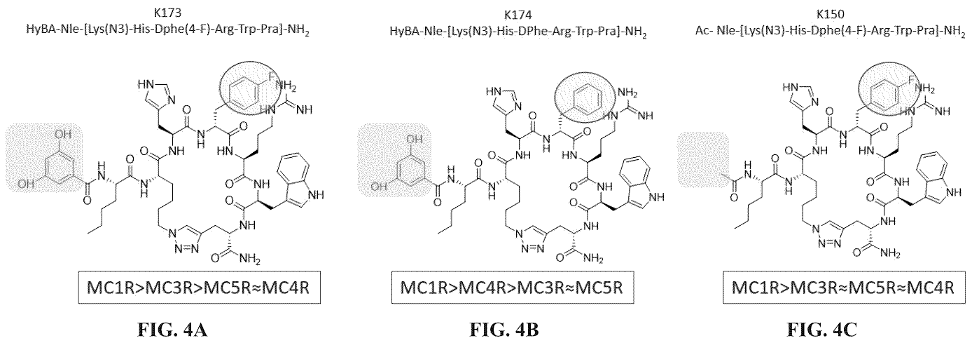

The K173 (KR-072), K174 (KR-073) and K150 (KR-049) peptides, the structures of which are depicted in FIGS. 4A, 4B and 4C, respectively, were each formulated in a 5 mg/mL sterile solution with HPMC, histidine buffer, and mannitol and stored at room temperature protected from light. The designated peptide formulations (35 μL) were administered to the eye of each animal using a calibrated pipette. With the animal manually restrained, the upper eyelid of the eye was gently elevated to expose the cornea. Treatment was then applied to the cornea without contacting the eye with the pipette tip. The animals were then allowed to blink several times while still manually restrained to distribute the applied solution over the eye prior to returning the animal to the cage. Treatment was administered twice daily (BID) with approximately 6-8 hours between each BID dose, with tissue collection one hour after the last dose. Tissues and blood collected include plasma, aqueous humor, iris/ciliary body, vitreous humor, eye cup containing the retinal pigment epithelium, choroid, sclera, cornea, retina, optic nerve, and brain.

Blood (3 mL) was collected IV or ICAR for terminal collections and placed into K2EDTA tubes for plasma acquisition. After whole blood was added, the tubes were gently mixed by inverting the tubes 5-8 times. Blood samples were stored on wet ice for up to 30 minutes prior to plasma processing. The samples were centrifuged at 4° C. for 10 minutes at 2000 g in a swinging bucket refrigerated centrifuge. After centrifugation, the clear plasma was transferred to prelabelled polypropylene tubes, snap frozen, and stored frozen at approximately-80° C.

Following euthanasia, eyes were enucleated and rinsed twice with fresh ice-cold PBS. Aqueous humor was removed via a 27 or 30 G syringe, weighed in polypropylene screw cap tubes, and snap frozen by immersing in liquid nitrogen. Eyes were dissected while frozen. All tissues were placed into individual vials, weighed, and snap frozen. The brain was collected and weighed. The hypothalamus, hippocampus and remaining brain was then dissected and weighed. 100-200 mg of each section was placed in pre-weighed polypropylene tubes, reweighed, and snap frozen. The remaining tissue was discarded. All tissues were stored frozen at −80° C. on dry ice.

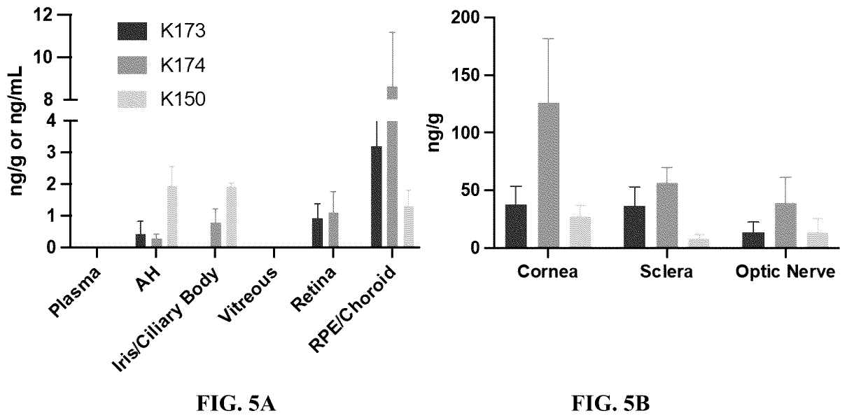

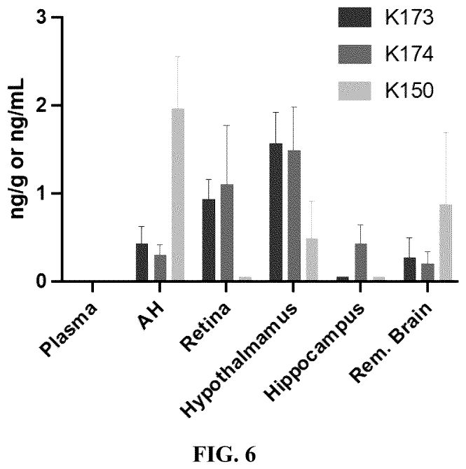

As shown in FIG. 5A and FIG. 5B, the K173 (KR-072) and K174 (KR-073) peptide formulations appeared to have a similar distribution pattern, while the K150 (KR-049) formulation demonstrated decreased scleral and retinal exposure and enhanced melanin binding. As shown in FIG. 6. AH levels for all three peptides were lower than that observed in the rabbit rat studies, and the K150 (KR-049) peptide demonstrated enhanced corneal permeability similar to that observed in the rabbit study. As also shown in FIG. 6, all three peptides were detected in the CNS of the animals, with no behavioral or metabolic changes noted in these animals despite the significant exposure. The foregoing results therefor confirm that the peptides disclosed herein can be delivered topically for retinal indications.

### Example 7. Potency and Solubility Comparison

An ideal MCR agonist candidate will have 100× potency over native peptide to MC1R. The MCR agonists in vivo ocular permeability should allow for concentrations well exceeding EC₅₀ values. The results of an assessment of MCR agonist candidates, including K110 (KR-009), K150 (KR-049), K172 (KR-071), K173 (KR-072), and K174 (KR-073), are presented below in Table 7.

TABLE 7
EC₅₀ Values

| | | | | | | | Topical |
|---|---|---|---|---|---|---|---|
| | | | | | | | Ocular |
| | EC₅₀(nM)- | EC₅₀(nM)- | EC₅₀(nM)- | EC₅₀(nM)- | | | Permeability |
| Peptide ID | MC1R | MC3R | MC4R | MC5R | Solubility | | (nM ± SEM) |
| αMSH | 0.3 | 193.47 | 112.38 | 173.83 | <1 | mg/mL | BQL |
| K110 | 0.011 | 44.10 | 45.71 | 140.4 | >5 | mg/mL | 1.43 ± 1.01 |
| K150 | 0.019 | 21.3 | 16.11 | 25.19 | 9.95 | mg/mL | 23.9 ± 10.8 |
| K172 | 0.0023 | 49.01 | 103.41 | 73.93 | 6.10 | mg/mL | 2.5 ± 0.2 |
| K173 | 0.0019 | 43.47 | 98.57 | 78.05 | 6.47 | mg/mL | 15.5 ± 2.6 |
| K174 | 0.0041 | 126.55 | 13.89 | 133.66 | 7.69 | mg/mL | 12.3 ± 3.8 |

### Example 8. Neuroprotective Properties of the α-MSH Peptide Analogues In-Vitro

To assess the cytoprotective properties of the α-MSH peptide analogues in-vitro the present inventors tested their modulatory influence on reactive oxygen species (ROS) production and caspase activation in response to cellular stress. For these in-vitro challenge assays, the present inventors induced cellular stress using tert-Butyl hydroperoxide (TBHP) or calcium ionophore (A23187), compounds which are respectively known to induce oxidative and metabolic stress. Oxidative stress was correlated to an increase of ROS within cells. While ROS are a natural by-product of normal cellular metabolism, overproduction can overwhelm the cellular antioxidant capacity, inducing macromolecular damage (e.g., by reacting with DNA, proteins, and lipids) which leads to disruption of thiol redox circuits. In turn, prolonged ROS exposure causes an activation of the apoptotic pathway, necrosis and eventually cell death. Within the current assays, the present inventors monitored ROS accumulation and subsequent caspase activity as readouts of cellular stress and eventual cell death.

### Materials/Methods:

Prior to each challenge assay, the iPSC-derived ocular stem cells (ABC-101) were thawed and plated at a density of 15,000-25,000 cells per well in 96-well plates and allowed to grow over a period of 48-72 hours in standard DMEM/F12+ HEPES media with growth factor supplements and human serum albumin (HAS). During the challenge period, cells were then exposed to varying concentrations of either A23187 or TBHP over a period of 1-18 hours, within non-supplemented DMEM/F12+ HEPES media. For each experiment the inventors subsequently added a dose-range of the α-MSH peptide analogues and assayed challenged vs. un-challenged wells for the generation of ROS and caspase expression. Both the ROS and caspase readouts were obtained by using commercially available reagent kits, which are based on a fluorescent indicator readout for each metric per the supplier's protocol.

### Apo-ONE® Homogeneous Caspase-3/7 Assay (Promega)

The Apo-ONE® Homogeneous Caspase-3/7 Assay includes a profluorescent caspase-3/7 consensus substrate, rhodamine 110 bis-(N—CBZ-1-aspartyl-1-glutamyl-1-valyl-aspartic acid amide) (Z-DEVD-R110), and an optimized bifunctional cell lysis/activity buffer, to allow for the detection of caspase activity in a homogenized format. Following incubation, cells were lysed using the kit buffer and Caspase-3/7 enzymatic activity is monitored using the DEVD peptide substrate, which upon cleavage on the C-terminal side of the aspartate residue becomes fluorescent when excited at a wavelength of 498 nm. The emission maximum is 521 nm. The amount of fluorescent product generated is representative of the amount of active Caspase-3/7 present in the sample. Readouts are routinely taken at 80 min and 150 min after incubation is started.

### ROS Assay (Cellular Reactive Oxygen Species Detection Assay Kit, Abcam)

This kit is a sensitive fluorometric one-step assay to detect intracellular reactive oxygen species (ROS) such as superoxide and hydroxyl radicals. Since the assay uses a cell-permeable dye, the readout can be performed within live cells and results in a red fluorescent signal (Ex/Em=520/605 nm), which is correlated to the amount of accumulated ROS within each well. Within the present protocol, the inventors read ROS accumulation over time, starting from 1 hour after incubation up to 4 hours post-incubation.

### CCK Assay (Cell Counting Kit-8, Dojindo)

In addition to the ROS and Caspase Assay, the present inventors also assessed overall cell viability within challenged and unchallenged wells using a general cell viability assessment. CCK is a widely used colorimetric assay for the determination of cell viability in cell proliferation and cytotoxicity assays. Within this assay, WST-8, a highly water-soluble and colorless tetrazolium salt, is reduced due to dehydrogenase activity in living cells, resulting in a yellow-color formazan dye. WST-8 is tissue soluble and can be detected using a fluorescence plate reader, which allows this assay to be performed on live cells. As fluorescent readouts are proportional to the number of cells per well, this assay can not only be used to determine cell viability post-challenge but was also employed to confirm uniform cell distribution amongst wells, prior to performing the above-mentioned assays (this relates to readouts, called CCK baseline within the appended excel tables).

### Results

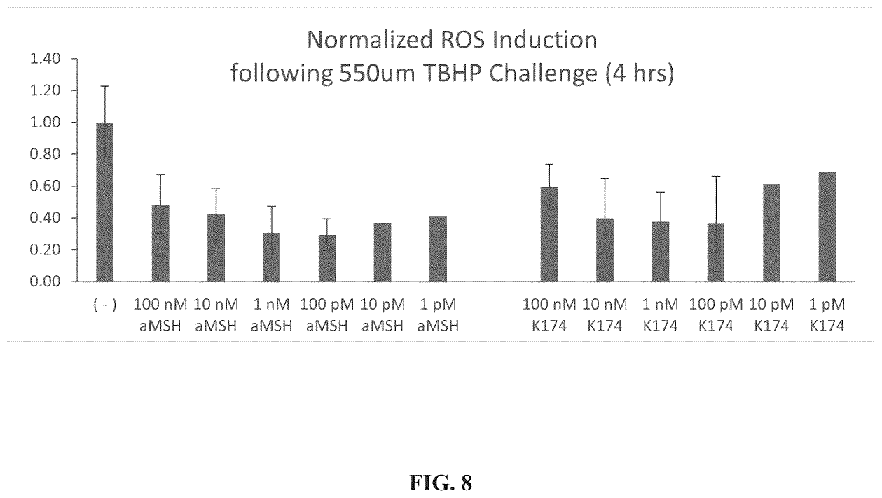

The ocular stem cells (ABC-101 cells) were challenged with 550 uM of TBHP and ROS production was assessed over several hours. FIGS. 7-8 respectively present summary data for ROS values at 3 hours and at 4 hours post-challenge induction, normalized to wells which were challenged without α-MSH peptide analogue addition. As shown in FIGS. 7-8, both the native α-MSH peptide as well as the α-MSH peptide analogue evaluated (K174, corresponding to SEQ ID NO: 73) are protective against ROS accumulation down to the low pM range. Their effectiveness appears to be comparable within the observed concentration range.

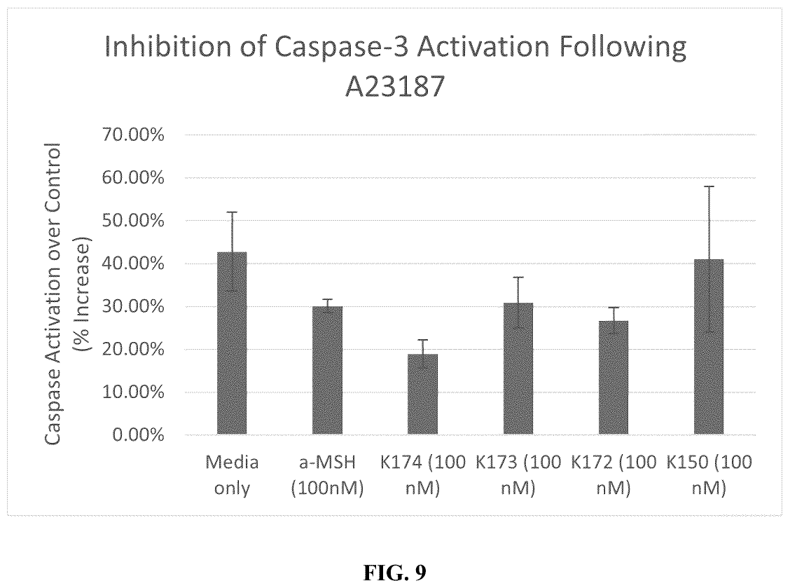

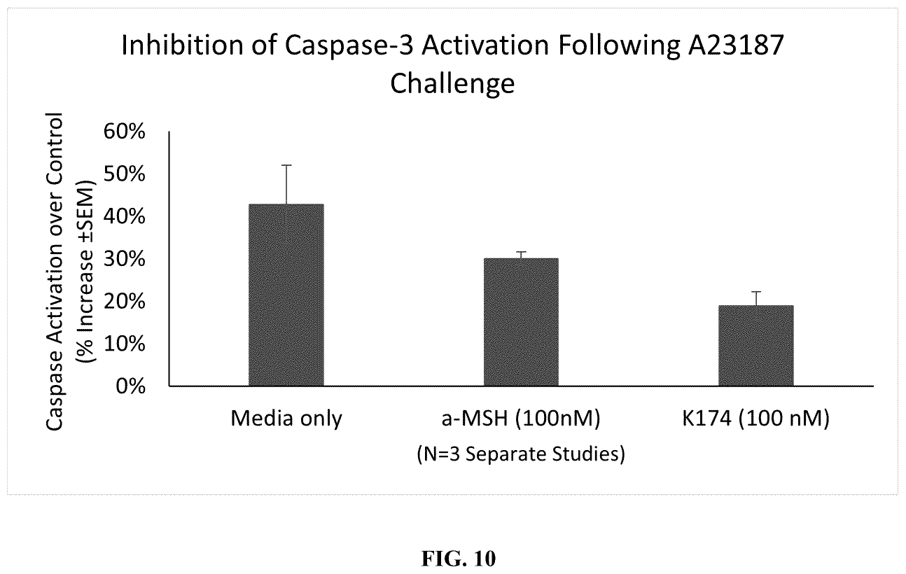

Following a similar layout as with the ROS induction assay, the present inventors subsequently assessed induction of Caspase 3/7 activity following a 1 hour challenge with 2.5 uM A23187. Readings from Caspase 3/7 activation were taken at 80 and 150 minutes post-substrate addition and readouts were compared to non-supplemented controls. Several of the α-MSH peptide analogues disclosed herein displayed a reduction of Caspase activity comparable to the native α-MSH peptide, with K174 (SEQ ID NO: 73) providing the highest degree of protection against apoptosis induction, as shown in FIGS. 9-10.

The foregoing examples and results confirm that the cytoprotective properties of the α-MSH peptide analogues disclosed herein to be at least comparable, if not superior to the native α-MSH peptide, at least as evidenced by the slowed accumulation of reactive oxygen species in response to cellular stress, and in turn a reduction of Caspase activation, leading to higher cell viability. The observed improvements in the biological activity of the α-MSH peptide analogues disclosed herein is complementary to their improved biochemical properties (e.g., improved solubility, improved permeability, etc.), which makes them superior drug substances as compared to the native α-MSH peptide. The foregoing in-vitro studies demonstrate that the α-MSH peptide analogues disclosed herein are able to enhance the survival of ocular stem cells (e.g., ABC-101 cells) post-transplantation, especially within hosts with on-going neurodegenerative conditions, by enhancing cellular resistance to oxidative and metabolic stressors.

### Example 9.—In Vivo Topical Administration Study—Rabbit

The objective of the present study was to evaluate the efficacy of the α-MSH peptide analogues in reducing corneal edema following descemetorhexis without endothethelial keratoplasty (DWEK) surgery in rabbits. The K173 (KR-072) peptide was formulated in a 1 mg/ml or 5 mg/ml sterile solution with HPMC, histidine buffer, and mannitol. The designated peptide formulations (35 μL) were administered to the eye of each animal using a calibrated pipette. With the animal manually restrained, the upper eyelid of the eye was gently elevated to expose the cornea. Treatment was then applied to the cornea without contacting the eye with the pipette tip. The animal was then allowed to blink several times while still manually restrained to distribute the applied solution over the eye prior to being returned to the cage. Treatment was administered once (QD) or twice daily (BID) with approximately 6-8 hours between each BID dose.

The rabbits were subjected to a surgical procedure prior to administering topical treatment. The animals received buprenorphine (0.01-0.05 mg/kg SC) and were sedated immediately prior to the procedure with ketamine/xylazine cocktail (20-50/4-10 mg/kg IM). Aseptic precautions were taken for the surgical procedure. Following induction of anesthesia, 5% betadine solution was used to clean the periocular (eyelids) area of the left eye. Betadine was then used to irrigate the ocular surface and conjunctival cul-de-sacs of the left eye. Sterile eyewash was then used to irrigate the ocular surface of the left eye. One drop each of 0.5% proparacaine HCL and 10% phenylephrine HCL was applied to the ocular surface of the left eye. The animal's corneal epithelium was marked with a circular ring (5 mm) to create a template for resection of the host tissue. A 2.4 mm keratome was used to create a superior incision. Descemet's membrane was scored peripherally using a reverse Terry-Sinskey hook, and then Descemet's membrane was peeled from the overlying stroma. Descemet's membrane was removed using forceps. Viscoelastic was removed by Irrigation Aspiration (IA) using balanced salt saline (BSS). One (1) ml of epinephrine 1:1000 and 0.5 ml of heparin (10,000 USP units/ml) was added to each 500 ml of irrigation solution (BSS) to facilitate pupil dilation, control inflammation, and reduce fibrin formation. Following closure with 9-0 nylon suture, BSS was injected to normalize the intraocular pressure. The rabbits were given a topical antibiotic (Neo-Poly Gramicidin) following surgery followed by topical Prednisolone Acetate (Prednisolone Acetate ophthalmic suspension, 1.0%), approximately 10 min after the antibiotic drop. The first dose of the test article was administered approximately 10 min after the Prednisolone dose. 4-6 hours after surgery, the rabbits were given another drop of Prednisolone Acetate and then BID for 7 days following surgery (AM and PM). The animals were also given topical antibiotics BID for 3 days following surgery. At least 5 minutes were allowed between Prednisolone Acetate and antibiotic drops. The test article was administered after the Prednisolone and antibiotic drops with at least 5 minutes separating the doses. Animals were given an additional dose of buprenorphine 4-6 hours after completion of the surgery.

Group 1 was administered a single dose, Group 2 received 4 doses, Group 3 received 8 BID doses. Samples were collected from 3 animals/group 30 min, 1 hr, 2 hr, 4 hr, and 8 hr post-last dose and from 2 animals/group 24 hours post-last dose.

A veterinary ophthalmologist performed complete ocular examinations using a slit lamp biomicroscope and indirect ophthalmoscope to evaluate ocular surface morphology and anterior and posterior segment inflammation on all animals prior to injection to serve as a baseline for enrollment into the study as well as on specific study days. Animals were not tranquilized for the examinations.

Following euthanasia, eyes were enucleated, rinsed 2× with fresh ice-cold PBS, and dabbed dry with gauze. Aqueous humor was removed via a 27- or 30-gauge syringe, placed into a pre-weighted 2 mL polypropylene tube, reweighed, and snap frozen by immersion in liquid nitrogen. The cornea was harvested fresh from each eye, placed into a pre-weighted re-enforced 2 mL polypropylene tube, re-weighed and snap frozen in liquid nitrogen. The remaining ocular tissues were discarded. Ocular samples were stored −80° C. All samples were placed into individual vials and weighed. Aqueous humor and cornea samples were collected.

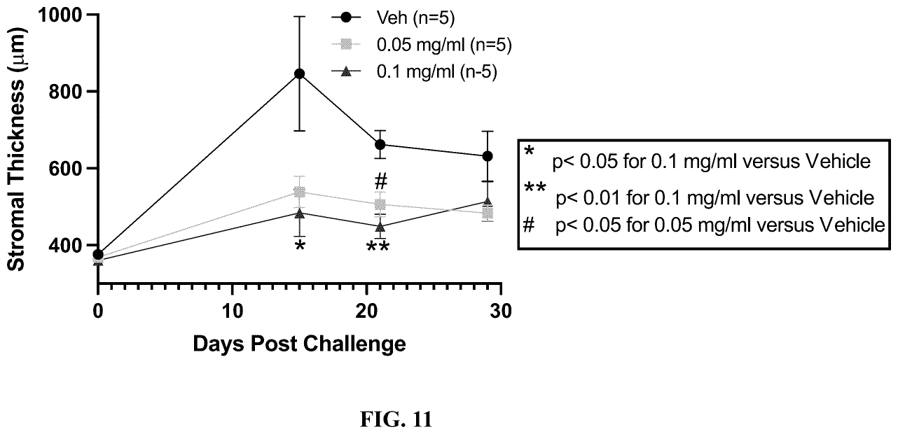

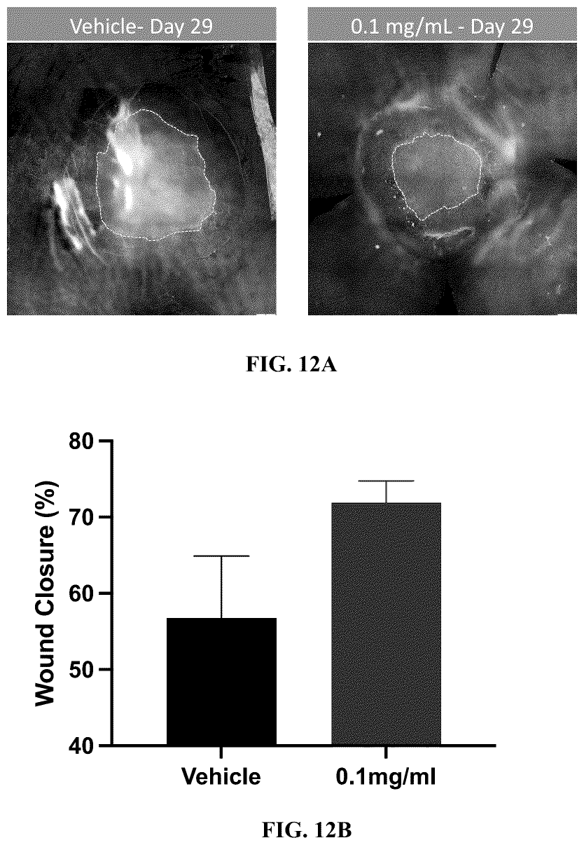

As depicted in FIG. 11, a decrease in corneal edema was observed. In particular, Statistically significant efficacy was observed soon after dosing with 2 lowest treatment arms when looking at corneal stromal thickness by OCT. A corneal flat mount analysis was performed on three animals per group to assess the rate of wound closure and, as shown in FIGS. 12A and 12B, the animals in the treatment group demonstrated enhanced wound healing.

### X. EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with reference to the appended claims so as to provide the broadest possible interpretation of such claims in view of the prior art and, therefore, to effectively encompass the intended scope of the disclosure.

1. A method of treatment comprising:
a) topically administering one or more of α-MSH peptide analogs to a subject in need thereof, wherein the one or more α-MSH peptide analogs are selected from the group consisting of SEQ ID NOs: 1-79; and
b) intraocularly administering a population of isolated cells to the subject, wherein the cells are selected from the group consisting of ocular stem cells, retinal ganglion precursor cells, retinal pigmented epithelial (RPE) cells, and corneal endothelial cells.

2. The method of claim 1, wherein the one or more of the α-MSH peptide analogs are administered topically or ophthalmologically to the subject for about 14 days prior to transplanting the population of cells into the subject.

3. The method of claim 1, wherein the population of cells are contacted in vitro or ex vivo with one or more α-MSH peptide analogs prior to being administered or transplanted in the subject, and wherein α-MSH peptide analogs are the selected from SEQ ID NOs: 1-79.

4. A cell therapy method comprising:
a) obtaining a population of cells from an organism or a cell source;
b) contacting the population of cells in vitro or ex vivo with one or more α-MSH peptide analogs selected from SEQ ID NOs: 1-79; and
c) transplanting the population of cells into a subject in need thereof.

5. The method of claim 4, wherein the one or more of the α-MSH peptide analogs are administered to the subject prior to transplanting the population of cells into the subject.

6. (canceled)

7. (canceled)

8. The method of claim 4, further comprising expanding the population of cells prior to or after contacting the population of cells with the one or more α-MSH peptide analogs of SEQ ID NOs: 1-79.

9. (canceled)

10. The method of claim 4, wherein the population of cells is treated with one or more α-MSH peptide analogs for less than 24 hours.

11. The method of claim 4, wherein the population of cells comprises one or more cell types selected from group consisting of a stem cell, a progenitor cell, and a somatic cell.

12. The method of claim 4, wherein the population of cells is autologous, allogeneic, or xenogeneic.

13. (canceled)

14. (canceled)

15. (canceled)

16. The method of claim 4, wherein the population of cells comprises ocular stem cells.

17. The method of claim 4, wherein the population of cells comprises cell-fate further restricted precursors of ocular stem cells.

18. (canceled)

19. (canceled)

20. (canceled)

21. (canceled)

22. The method of claim 4, wherein the population of cells is engineered to express at least one payload.

23. The method of claim 22, wherein the at least one payload of interest is one or more of a protein of interest, a fusion polypeptide, an antibody, an antigen, a chimeric antigen receptor (CAR), a T cell receptor (TCR), a safety switch, and/or a regulatory switch.

24. The method of claim 4, wherein the population of cells is genetically modified using a gene editing system selected from the group consisting of a CRISPR, a TALEN, a Zn-Finger, and a vector delivery system.

25. (canceled)

26. (canceled)

27. The method of claim 4, wherein the population of cells are activated prior to transplanting the population of cells.

28. The method of claim 4, wherein the population of cells expresses MCRs.

29. The method of claim 4, wherein the α-MSH peptide analog is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, and SEQ ID NO: 75.

30. The method of claim 4, wherein the step of contacting the population of cells in vitro or ex vivo with one or more α-MSH peptide analogs comprises engineering the population of cells to create an engineered cell capable of endogenously expressing a transgene encoding the one or more α-MSH peptide analogs.

31-36. (canceled)

37. A method of preparing a therapeutic composition comprising engineered ocular cells comprising:
a) providing an isolated ocular cell;
b) contacting the ocular cell with a transgene expression vector in vitro, thereby introducing the expression vector into the ocular cells to form engineered ocular cells, wherein the transgene encodes an α-MSH polypeptide, wherein the transgene expression vector comprises cis-regulatory and promoter sequences that control the expression of the transgene; and
c) formulating the engineered ocular cells into a suspension for intraocular administration.

38.-45. (canceled)

46. A therapeutic composition for treating an ocular disease or disorder in a human subject in need thereof, comprising:
ocular cells that are introduced with a transgene expression vector in vitro,
wherein the transgene expression vector comprises cis-regulatory and promoter sequences that control the expression of a transgene encoding an α-MSH polypeptide; and
wherein the ocular cells comprising the transgene are formulated into a suspension of cells for intraocular administration to the human subject.

47-58. (canceled)


SEQUENCE LISTING (170 sequences)

SEQ ID NO: 1 - protein, 12 aa, synthetic construct
  SITE 1: note = Acetyl tyrosine
  SITE 12: note = valine amide
YSMEHFRWGKPV

SEQ ID NO: 2 - protein, 11 aa, synthetic construct
  SITE 1: note = Acetyl serine
  SITE 11: note = valine amide
SMEHFRWGKPV

SEQ ID NO: 3 - protein, 10 aa, synthetic construct
  SITE 1: note = Acetyl methionine
  SITE 10: note = valine amide
MEHFRWGKPV

SEQ ID NO: 4 - protein, 9 aa, synthetic construct
  SITE 1: note = Acetyl glutamic acid
  SITE 9: note = valine amide
EHFRWGKPV

SEQ ID NO: 5 - protein, 12 aa, synthetic construct
  SITE 1: note = Acetyl serine
  SITE 12: note = proline amide
SYSMEHFRWGKP

SEQ ID NO: 6 - protein, 11 aa, synthetic construct
  SITE 1: note = Acetyl serine
  SITE 11: note = lysine amide
SYSMEHFRWGK

SEQ ID NO: 7 - protein, 8 aa, synthetic construct
  SITE 1: note = Acetyl glutamic acid
  SITE 8: note = proline amide
EHFRWGKP

SEQ ID NO: 8 - protein, 7 aa, synthetic construct
  SITE 1: note = Acetyl norleucine
  SITE 2: note = (s)-2-amino-6-azidohexanoic acid
  SITE 7: note = propargyl-glycineamide
  SITE 2..7: note = triazole bridge
XKHFRWX

SEQ ID NO: 9 - protein, 7 aa, synthetic construct
  SITE 1: note = Acetyl norleucine
  SITE 2: note = (s)-2-amino-6-azidohexanoic acid
  SITE 4: note = D-phenylalanine
  SITE 7: note = propargyl-glycineamide
  SITE 2..7: note = triazole bridge
XKHFRWX

SEQ ID NO: 10 - protein, 8 aa, synthetic construct
  SITE 1: note = N2-acetyl-N6-diazo-L-lysine
  SITE 8: note = propargyl-glycineamide
  SITE 1..8: note = triazole bridge
KEHFRWGX

SEQ ID NO: 11 - protein, 6 aa, synthetic construct
  SITE 1: note = acetyl glutamic acid
  SITE 6: note = cysteinamide
  SITE 1..6: note = thioether bridge
EHFRWC

SEQ ID NO: 12 - protein, 7 aa, synthetic construct
  SITE 1: note = acetyl glutamic acid
  SITE 7: note = cysteinamide
  SITE 1..7: note = thioether bridge
EHFRWGC

SEQ ID NO: 13 - protein, 8 aa, synthetic construct
  SITE 1: note = Acetyl R-2-(4-pentenyl)alanine
  SITE 8: note = S-2-(4-pentenyl) alanine amide
  SITE 1..8: note = hydrocarbon staple
XEHFRWGX

SEQ ID NO: 14 - protein, 8 aa, synthetic construct
  SITE 1: note = Acetyl S-2-(4-pentenyl) alanine
  SITE 8: note = S-2-(4-pentenyl) alanine amide
  SITE 1..8: note = hydrocarbon staple
XEHFRWGX

SEQ ID NO: 15 - protein, 8 aa, synthetic construct
  SITE 1: note = Acetyl R-2-(7-octenyl) alanine
  SITE 8: note = S-2-(4-pentenyl) alanine amide
  SITE 1..8: note = hydrocarbon staple
XEHFRWGX

SEQ ID NO: 16 - protein, 13 aa, synthetic construct
  SITE 1: note = 4-phenyl-butanoic acid serine
  SITE 13: note = valine amide
SYSMEHFRWGKPV

SEQ ID NO: 17 - protein, 15 aa, synthetic construct
  SITE 1: note = Octanoyl polyethylene glycol-8 glycine
  SITE 15: note = valine amide
GGSYSMEHFRWGKPV

SEQ ID NO: 18 - protein, 13 aa, synthetic construct
  SITE 1: note = 4-pyridylacetyl acid serine
  SITE 13: note = valine amide
SYSMEHFRWGKPV

SEQ ID NO: 19 - protein, 13 aa, synthetic construct
  SITE 1: note = 3,5-hydroxybenzoic acid serine
  SITE 13: note = valine amide
SYSMEHFRWGKPV

SEQ ID NO: 20 - protein, 13 aa, synthetic construct
  SITE 1: note = Octanoyl serine
  SITE 13: note = valine amide
SYSMEHFRWGKPV

SEQ ID NO: 21 - protein, 13 aa, synthetic construct
  SITE 1: note = Acetyl serine
  SITE 6: note = N-methyl histidine
  SITE 13: note = valine amide
SYSMEHFRWGKPV

SEQ ID NO: 22 - protein, 13 aa, synthetic construct
  SITE 1: note = Acetyl serine
  SITE 7: note = N-methyl phenylalanine
  SITE 13: note = valine amide
SYSMEHFRWGKPV

SEQ ID NO: 23 - protein, 13 aa, synthetic construct
  SITE 1: note = Acetyl serine
  SITE 8: note = N-methyl arginine
  SITE 13: note = valine amide
SYSMEHFRWGKPV

SEQ ID NO: 24 - protein, 13 aa, synthetic construct
  SITE 1: note = Acetyl serine
  SITE 9: note = N-methyl tryptophan
  SITE 13: note = valine amide
SYSMEHFRWGKPV

SEQ ID NO: 25 - protein, 13 aa, synthetic construct
  SITE 1: note = Acetyl serine
  SITE 7: note = N-methyl-D-phenylalanine
  SITE 13: note = valine amide
SYSMEHFRWGKPV

SEQ ID NO: 26 - protein, 7 aa, synthetic construct
  SITE 1: note = Acetyl norleucine
  SITE 2: note = R-2-(4-pentenyl)alanine
  SITE 4: note = D-phenylalanine
  SITE 6: note = S-2-(4-pentenyl) alanine
  SITE 7: note = tryptophan amide
XXHFRXW

SEQ ID NO: 27 - protein, 7 aa, synthetic construct
  SITE 1: note = Acetyl norleucine
  SITE 2: note = N-(2-carboxyethyl)glycine
  SITE 4: note = D-phenylalanine
  SITE 6: note = Diaminobuyric acid
  SITE 7: note = tryptophan amide
XXHFRXW

SEQ ID NO: 28 - protein, 7 aa, synthetic construct
  SITE 1: note = Acetyl norleucine
  SITE 4: note = D-phenylalanine
  SITE 6: note = N-(2-aminoethyl) glycine
  SITE 7: note = tryptophan amide
XEHFRXW

SEQ ID NO: 29 - protein, 7 aa, synthetic construct
  SITE 1: note = Acetyl norleucine
  SITE 2: note = N-(2-carboxyethyl)glycine
  SITE 4: note = D-phenylalanine
  SITE 6: note = N-(2-aminoethyl) glycine
  SITE 7: note = tryptophan amide
XXHFRXW

SEQ ID NO: 30 - protein, 7 aa, synthetic construct
  SITE 1: note = Acetyl norleucine
  SITE 2: note = N-(2-aminoethyl) glycine
  SITE 4: note = D-phenylalanine
  SITE 6: note = N-(2-aminoethyl) glycine
XXHFRXW

SEQ ID NO: 31 - protein, 7 aa, synthetic construct
  SITE 1: note = Acetyl norleucine
  SITE 2: note = alpha methyl cysteine
  SITE 4: note = D-phenylalanine
  SITE 6: note = alpha methyl cysteine
  SITE 7: note = tryptophan amide
XCHFRCW

SEQ ID NO: 32 - protein, 7 aa, synthetic construct
  SITE 1: note = Acetyl norleucine
  SITE 4: note = D-phenylalanine
XCHFRCW

SEQ ID NO: 33 - protein, 8 aa, synthetic construct
  SITE 1: note = Acetyl R-2-(7-octenyl) alanine
  SITE 4: note = D-phenylalanine
  SITE 8: note = S-2-(7-octenyl) alanine amide
  SITE 1..8: note = hydrocarbon staple
XEHFRWGX

SEQ ID NO: 34 - protein, 8 aa, synthetic construct
  SITE 1: note = Acetyl norleucine
  SITE 2: note = R-2-(7-octenyl) alanine
  SITE 4: note = D-phenylalanine
  SITE 7: note = S-2-(4-pentenyl) alanine
  SITE 8: note = glycineamide
  SITE 2..7: note = hydrocarbon staple
XXHFRWXG

SEQ ID NO: 35 - protein, 8 aa, synthetic construct
  SITE 1: note = Acetyl norleucine
  SITE 8: note = lysine amide
XEHFRWGK

SEQ ID NO: 36 - protein, 9 aa, synthetic construct
  SITE 1: note = acetyl norleucine
  SITE 9: note = cysteinamide
XEHFRWGKC

SEQ ID NO: 37 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 38 - protein, 7 aa, synthetic construct
  SITE 1: note = Acetyl norleucine
  SITE 2: note = R-2-(4-pentenyl)alanine
  SITE 4: note = D-phenylalanine
  SITE 7: note = S-2-(4-pentenyl) alanine amide
XXHFRWX

SEQ ID NO: 39 - protein, 7 aa, synthetic construct
  SITE 1: note = Acetyl norleucine
  SITE 2: note = S-2-(4-pentenyl) alanine
  SITE 4: note = D-phenylalanine
  SITE 7: note = S-2-(4-pentenyl) alanine amide
XXHFRWX

SEQ ID NO: 40 - protein, 7 aa, synthetic construct
  SITE 1: note = Acetyl norleucine
  SITE 4: note = D-phenylalanine
XCHFRWC

SEQ ID NO: 41 - protein, 7 aa, synthetic construct
  SITE 1: note = Acetyl norleucine
  SITE 2: note = Diaminobuyric acid
  SITE 4: note = D-phenylalanine
  SITE 7: note = Diaminobuyric acid
XXHFRWX

SEQ ID NO: 42 - protein, 7 aa, synthetic construct
  SITE 1: note = Acetyl norleucine
  SITE 2: note = (S)-2-Amino-4-azidobutanoic acid
  SITE 4: note = D-phenylalanine
  SITE 7: note = (S)-2-Amino-4-azidobutanoic acid
XXHFRWX

SEQ ID NO: 43 - protein, 7 aa, synthetic construct
  SITE 1: note = Acetyl norleucine
  SITE 2: note = (s)-2-amino-6-azidohexanoic acid
  SITE 3: note = N-methyl histidine
  SITE 4: note = D-phenylalanine
  SITE 7: note = propargyl-glycineamide
XKHFRWX

SEQ ID NO: 44 - protein, 7 aa, synthetic construct
  SITE 1: note = Acetyl norleucine
  SITE 2: note = (s)-2-amino-6-azidohexanoic acid
  SITE 4: note = N-methyl D-phenylalanine
  SITE 7: note = propargyl-glycineamide
XKHFRWX

SEQ ID NO: 45 - protein, 7 aa, synthetic construct
  SITE 1: note = Acetyl norleucine
  SITE 2: note = (s)-2-amino-6-azidohexanoic acid
  SITE 4: note = D-phenylalanine
  SITE 5: note = N-methyl arginine
  SITE 7: note = propargyl-glycineamide
XKHFRWX

SEQ ID NO: 46 - protein, 7 aa, synthetic construct
  SITE 1: note = Acetyl norleucine
  SITE 2: note = (s)-2-amino-6-azidohexanoic acid
  SITE 4: note = D-phenylalanine
  SITE 6: note = N-methyl tryptophan
  SITE 7: note = propargyl-glycineamide
XKHFRWX

SEQ ID NO: 47 - protein, 7 aa, synthetic construct
  SITE 1: note = Acetyl norleucine
  SITE 2: note = (s)-2-amino-6-azidohexanoic acid
  SITE 3: note = 4-amino-phenylalanine
  SITE 4: note = D-phenylalanine
  SITE 7: note = propargyl-glycineamide
XKFFRWX

SEQ ID NO: 48 - protein, 7 aa, synthetic construct
  SITE 1: note = Acetyl norleucine
  SITE 2: note = (s)-2-amino-6-azidohexanoic acid
  SITE 4: note = 4-trifluoromethyl-phenylalanine
  SITE 7: note = propargyl-glycineamide
XKHFRWX

SEQ ID NO: 49 - protein, 7 aa, synthetic construct
  SITE 1: note = Acetyl norleucine
  SITE 2: note = (s)-2-amino-6-azidohexanoic acid
  SITE 4: note = 4-fluoro-D-phenylalanine
  SITE 7: note = propargyl-glycineamide
XKHFRWX

SEQ ID NO: 50 - protein, 7 aa, synthetic construct
  SITE 1: note = Acetyl norleucine
  SITE 2: note = (s)-2-amino-6-azidohexanoic acid
  SITE 4: note = D-phenylalanine
  SITE 5: note = N-methyl arginine
  SITE 6: note = N-methyl tryptophan
  SITE 7: note = propargyl-glycineamide
XKHFRWX

SEQ ID NO: 51 - protein, 7 aa, synthetic construct
  SITE 1: note = Acetyl norleucine
  SITE 2: note = (s)-2-amino-6-azidohexanoic acid
  SITE 3: note = N-methyl histidine
  SITE 4: note = D-phenylalanine
  SITE 6: note = N-methyl tryptophan
  SITE 7: note = propargyl-glycineamide
XKHFRWX

SEQ ID NO: 52 - protein, 7 aa, synthetic construct
  SITE 1: note = Acetyl norleucine
  SITE 2: note = (s)-2-amino-6-azidohexanoic acid
  SITE 3: note = N-methyl histidine
  SITE 4: note = D-phenylalanine
  SITE 5: note = N-methyl arginine
  SITE 7: note = propargyl-glycineamide
XKHFRWX

SEQ ID NO: 53 - protein, 8 aa, synthetic construct
  SITE 1: note = 3,5-hydroxybenzoic acid norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 54 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 5: note = Homoarginine
  SITE 7: note = propargylglycine
  SITE 8: note = lysine amide
XKHFRWXK

SEQ ID NO: 55 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 5: note = N-omega,N'-omega-dimethyl-L-arginine
  SITE 7: note = propargylglycine
  SITE 8: note = lysine amide
XKHFRWXK

SEQ ID NO: 56 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = propargylglycine
  SITE 8: note = asparagine amide
XKHFRWXN

SEQ ID NO: 57 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = propargylglycine
  SITE 8: note = homoglutaminamide
XKHFRWXQ

SEQ ID NO: 58 - protein, 8 aa, synthetic construct
  SITE 1: note = Acetyl norleucine
  SITE 2: note = (s)-2-amino-6-azidohexanoic acid
  SITE 4: note = O-methyl-D-tyrosine
  SITE 7: note = propargylglycine
  SITE 8: note = lysine amide
XKHYRWXK

SEQ ID NO: 59 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = propargylglycine
  SITE 8: note = N-omega-dimethyl-lysine amide
XKHFRWXK

SEQ ID NO: 60 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 6: note = 1-methyl-tryptophan
  SITE 7: note = propargylglycine
  SITE 8: note = lysine amide
XKHFRWXK

SEQ ID NO: 61 - protein, 8 aa, synthetic construct
  SITE 1: note = Acetyl norleucine
  SITE 2: note = (s)-2-amino-6-azidohexanoic acid
  SITE 3: note = O-methyl-tyrosine
  SITE 4: note = D-phenylalanine
  SITE 7: note = propargylglycine
  SITE 8: note = lysine amide
XKXFRWXK

SEQ ID NO: 62 - protein, 8 aa, synthetic construct
  SITE 1: note = 3,5-dihydroxyphenylacetic acid norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 63 - protein, 8 aa, synthetic construct
  SITE 1: note = (3-methoxyphenyl)acetic acid (3-methoxyphenyl)acetic acid norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 64 - protein, 8 aa, synthetic construct
  SITE 1: note = 3-hydroxy-5-methoxybenzeneacetic acid 3-hydroxy-5-methoxybenzeneacetic acid norleucine
  SITE 2: note = (s)-2-amino-6-azidohexanoic acid
  SITE 4: note = D-phenylalanine
  SITE 7: note = propargylglycine
  SITE 8: note = lysine amide
XKHFRWXK

SEQ ID NO: 65 - protein, 8 aa, synthetic construct
  SITE 1: note = Acetyl norleucine
  SITE 2: note = (S)-2-amino-5-azidopentanoic acid
  SITE 4: note = D-phenylalanine
  SITE 7: note = propargylglycine
  SITE 8: note = lysine amide
XXHFRWXK

SEQ ID NO: 66 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = homopropargylglycine
  SITE 8: note = lysine amide
XKHFRWXK

SEQ ID NO: 67 - protein, 8 aa, synthetic construct
  SITE 1: note = Acetyl norleucine
  SITE 2: note = propargylglycine
  SITE 4: note = D-phenylalanine
  SITE 7: note = (s)-2-amino-6-azidohexanoic acid
  SITE 8: note = lysine amide
XXHFRWKK

SEQ ID NO: 68 - protein, 8 aa, synthetic construct
  SITE 1: note = Acetyl norleucine
  SITE 2: note = (S)-2-amino-5-azidopentanoic acid
  SITE 4: note = D-phenylalanine
  SITE 7: note = homopropargylglycine
  SITE 8: note = lysine amide
XXHFRWXK

SEQ ID NO: 69 - protein, 8 aa, synthetic construct
  SITE 1: note = Acetyl norleucine
  SITE 2: note = (R)-2-amino-6-azidohexanoic acid
  SITE 4: note = D-phenylalanine
  SITE 7: note = propargylglycine
  SITE 8: note = lysine amide
XKHFRWXK

SEQ ID NO: 70 - protein, 8 aa, synthetic construct
  SITE 1: note = Acetyl norleucine
  SITE 2: note = (s)-2-amino-6-azidohexanoic acid
  SITE 4: note = D-phenylalanine
  SITE 7: note = D-propargylglycine
  SITE 8: note = lysine amide
XKHFRWXK

SEQ ID NO: 71 - protein, 8 aa, synthetic construct
  SITE 1: note = 3,5-hydroxybenzoic acid norleucine
  SITE 2: note = (s)-2-amino-6-azidohexanoic acid
  SITE 4: note = 4-fluoro-D-phenylalanine
  SITE 7: note = propargylglycine
  SITE 8: note = homoglutaminamide
XKHFRWXQ

SEQ ID NO: 72 - protein, 7 aa, synthetic construct
  SITE 1: note = 3,5-hydroxybenzoic acid norleucine
  SITE 2: note = (s)-2-amino-6-azidohexanoic acid
  SITE 4: note = 4-fluoro-D-phenylalanine
  SITE 7: note = propargyl-glycineamide
XKHFRWX

SEQ ID NO: 73 - protein, 7 aa, synthetic construct
  SITE 1: note = 3,5-hydroxybenzoic acid norleucine
  SITE 2: note = (s)-2-amino-6-azidohexanoic acid
  SITE 4: note = D-phenylalanine
  SITE 7: note = propargyl-glycineamide
XKHFRWX

SEQ ID NO: 74 - protein, 7 aa, synthetic construct
  SITE 1: note = 4-pyridylacetyl acid norleucine
  SITE 2: note = (s)-2-amino-6-azidohexanoic acid
  SITE 4: note = D-phenylalanine
  SITE 7: note = propargyl-glycineamide
XKHFRWX

SEQ ID NO: 75 - protein, 7 aa, synthetic construct
  SITE 1: note = 3,5-hydroxybenzoic acid norleucine
  SITE 2: note = (s)-2-amino-6-azidohexanoic acid
  SITE 3: note = N-methyl histidine
  SITE 4: note = 4-fluoro-D-phenylalanine
  SITE 6: note = N-methyl tryptophan
  SITE 7: note = propargyl-glycineamide
XKHFRWX

SEQ ID NO: 76 - protein, 13 aa, synthetic construct
  SITE 1: note = Acetyl serine
  SITE 13: note = valine amide
SYSMEHFRWGKPV

SEQ ID NO: 77 - protein, 13 aa, synthetic construct
  SITE 1: note = Acetyl serine
  SITE 4: note = Norleucine
  SITE 7: note = D-phenylalanine
  SITE 13: note = valine amide
SYSXEHFRWGKPV

SEQ ID NO: 78 - protein, 39 aa, synthetic construct
  SITE 1: note = Acetyl serine
  SITE 39: note = phenylalanine amide
SYSMEHFRWGKPVGKKRRPVKVYPNGAEDESAEAFPLEF

SEQ ID NO: 79 - protein, 7 aa, synthetic construct
  SITE 1: note = Acetyl norleucine
  SITE 4: note = D-phenylalanine
  SITE 6: note = Diaminobuyric acid
  SITE 7: note = tryptophan amide
XEHFRXW

SEQ ID NO: 80 - protein, 266 aa, Homo sapiens
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 81 - protein, 7 aa, synthetic construct
MEHFRWG

SEQ ID NO: 82 - protein, 4 aa, synthetic construct
HFRW

SEQ ID NO: 83 - protein, 10 aa, synthetic construct
  SITE 1: note = Acetyl serine
  SITE 10: note = glycineamide
SYSMEHFRWG

SEQ ID NO: 84 - protein, 9 aa, synthetic construct
  SITE 1: note = Acetyl serine
  SITE 9: note = tryptophan amide
SYSMEHFRW

SEQ ID NO: 85 - protein, 11 aa, synthetic construct
  SITE 1: note = Acetyl tyrosine
  SITE 11: note = proline amide
YSMEHFRWGKP

SEQ ID NO: 86 - protein, 10 aa, synthetic construct
  SITE 1: note = Acetyl tyrosine
  SITE 10: note = lysine amide
YSMEHFRWGK

SEQ ID NO: 87 - protein, 9 aa, synthetic construct
  SITE 1: note = Acetyl tyrosine
  SITE 9: note = glycineamide
YSMEHFRWG

SEQ ID NO: 88 - protein, 8 aa, synthetic construct
  SITE 1: note = Acetyl tyrosine
  SITE 8: note = tryptophan amide
YSMEHFRW

SEQ ID NO: 89 - protein, 10 aa, synthetic construct
  SITE 1: note = Acetyl serine
  SITE 10: note = proline amide
SMEHFRWGKP

SEQ ID NO: 90 - protein, 9 aa, synthetic construct
  SITE 1: note = Acetyl serine
  SITE 9: note = lysine amide
SMEHFRWGK

SEQ ID NO: 91 - protein, 8 aa, synthetic construct
  SITE 1: note = Acetyl serine
  SITE 8: note = glycineamide
SMEHFRWG

SEQ ID NO: 92 - protein, 7 aa, synthetic construct
  SITE 1: note = Acetyl serine
  SITE 7: note = tryptophan amide
SMEHFRW

SEQ ID NO: 93 - protein, 9 aa, synthetic construct
  SITE 1: note = Acetyl methionine
  SITE 9: note = proline amide
MEHFRWGKP

SEQ ID NO: 94 - protein, 8 aa, synthetic construct
  SITE 1: note = Acetyl methionine
  SITE 8: note = lysine amide
MEHFRWGK

SEQ ID NO: 95 - protein, 7 aa, synthetic construct
  SITE 1: note = Acetyl methionine
  SITE 7: note = glycineamide
MEHFRWG

SEQ ID NO: 96 - protein, 6 aa, synthetic construct
  SITE 1: note = Acetyl methionine
  SITE 6: note = tryptophan amide
MEHFRW

SEQ ID NO: 97 - protein, 7 aa, synthetic construct
  SITE 1: note = Acetyl glutamic acid
  SITE 7: note = lysine amide
EHFRWGK

SEQ ID NO: 98 - protein, 6 aa, synthetic construct
  SITE 1: note = Acetyl glutamic acid
  SITE 6: note = glycineamide
EHFRWG

SEQ ID NO: 99 - protein, 5 aa, synthetic construct
  SITE 1: note = Acetyl glutamic acid
  SITE 5: note = tryptophan amide
EHFRW

SEQ ID NO: 100 - protein, 8 aa, synthetic construct
  SITE 1: note = Acetyl histidine
  SITE 8: note = valine amide
HFRWGKPV

SEQ ID NO: 101 - protein, 7 aa, synthetic construct
  SITE 1: note = Acetyl histidine
  SITE 7: note = proline amide
HFRWGKP

SEQ ID NO: 102 - protein, 6 aa, synthetic construct
  SITE 1: note = Acetyl histidine
  SITE 6: note = lysine amide
HFRWGK

SEQ ID NO: 103 - protein, 5 aa, synthetic construct
  SITE 1: note = Acetyl histidine
  SITE 5: note = glycineamide
HFRWG

SEQ ID NO: 104 - protein, 4 aa, synthetic construct
  SITE 1: note = Acetyl histidine
  SITE 4: note = tryptophan amide
HFRW

SEQ ID NO: 105 - protein, 6 aa, synthetic construct
LLLLLL

SEQ ID NO: 106 - protein, 6 aa, synthetic construct
GLLLLL

SEQ ID NO: 107 - protein, 6 aa, synthetic construct
LGLLLL

SEQ ID NO: 108 - protein, 6 aa, synthetic construct
LLGLLL

SEQ ID NO: 109 - protein, 6 aa, synthetic construct
LLLGLL

SEQ ID NO: 110 - protein, 6 aa, synthetic construct
LLLLGL

SEQ ID NO: 111 - protein, 6 aa, synthetic construct
LLLLLG

SEQ ID NO: 112 - protein, 6 aa, synthetic construct
GGLLLL

SEQ ID NO: 113 - protein, 6 aa, synthetic construct
GLGLLL

SEQ ID NO: 114 - protein, 6 aa, synthetic construct
GLLGLL

SEQ ID NO: 115 - protein, 6 aa, synthetic construct
GLLLGL

SEQ ID NO: 116 - protein, 6 aa, synthetic construct
GLLLLG

SEQ ID NO: 117 - protein, 6 aa, synthetic construct
LGGLLL

SEQ ID NO: 118 - protein, 6 aa, synthetic construct
LGLGLL

SEQ ID NO: 119 - protein, 6 aa, synthetic construct
LGLLGL

SEQ ID NO: 120 - protein, 6 aa, synthetic construct
LGLLLG

SEQ ID NO: 121 - protein, 6 aa, synthetic construct
LLGGLL

SEQ ID NO: 122 - protein, 6 aa, synthetic construct
LLGLGL

SEQ ID NO: 123 - protein, 6 aa, synthetic construct
LLGLLG

SEQ ID NO: 124 - protein, 6 aa, synthetic construct
LLLGGL

SEQ ID NO: 125 - protein, 6 aa, synthetic construct
LLLGLG

SEQ ID NO: 126 - protein, 6 aa, synthetic construct
LLLLGG

SEQ ID NO: 127 - protein, 6 aa, synthetic construct
GGGLLL

SEQ ID NO: 128 - protein, 6 aa, synthetic construct
GGLGLL

SEQ ID NO: 129 - protein, 6 aa, synthetic construct
GGLLGL

SEQ ID NO: 130 - protein, 6 aa, synthetic construct
GGLLLG

SEQ ID NO: 131 - protein, 6 aa, synthetic construct
GLGGLL

SEQ ID NO: 132 - protein, 6 aa, synthetic construct
GLGLGL

SEQ ID NO: 133 - protein, 6 aa, synthetic construct
GLGLLG

SEQ ID NO: 134 - protein, 6 aa, synthetic construct
GLLGGL

SEQ ID NO: 135 - protein, 6 aa, synthetic construct
GLLGLG

SEQ ID NO: 136 - protein, 6 aa, synthetic construct
GLLLGG

SEQ ID NO: 137 - protein, 6 aa, synthetic construct
LLLGGG

SEQ ID NO: 138 - protein, 6 aa, synthetic construct
LLGLGG

SEQ ID NO: 139 - protein, 6 aa, synthetic construct
LLGGLG

SEQ ID NO: 140 - protein, 6 aa, synthetic construct
LLGGGL

SEQ ID NO: 141 - protein, 6 aa, synthetic construct
LGLLGG

SEQ ID NO: 142 - protein, 6 aa, synthetic construct
LGLGLG

SEQ ID NO: 143 - protein, 6 aa, synthetic construct
LGLGGL

SEQ ID NO: 144 - protein, 6 aa, synthetic construct
LGGLLG

SEQ ID NO: 145 - protein, 6 aa, synthetic construct
LGGLGL

SEQ ID NO: 146 - protein, 6 aa, synthetic construct
LGGGLL

SEQ ID NO: 147 - protein, 6 aa, synthetic construct
LLGGGG

SEQ ID NO: 148 - protein, 6 aa, synthetic construct
LGLGGG

SEQ ID NO: 149 - protein, 6 aa, synthetic construct
LGGLGG

SEQ ID NO: 150 - protein, 6 aa, synthetic construct
LGGGLG

SEQ ID NO: 151 - protein, 6 aa, synthetic construct
LGGGGL

SEQ ID NO: 152 - protein, 6 aa, synthetic construct
GLLGGG

SEQ ID NO: 153 - protein, 6 aa, synthetic construct
GLGLGG

SEQ ID NO: 154 - protein, 6 aa, synthetic construct
GLGGLG

SEQ ID NO: 155 - protein, 6 aa, synthetic construct
GLGGGL

SEQ ID NO: 156 - protein, 6 aa, synthetic construct
GGLLGG

SEQ ID NO: 157 - protein, 6 aa, synthetic construct
GGLGLG

SEQ ID NO: 158 - protein, 6 aa, synthetic construct
GGLGGL

SEQ ID NO: 159 - protein, 6 aa, synthetic construct
GGGLLG

SEQ ID NO: 160 - protein, 6 aa, synthetic construct
GGGLGL

SEQ ID NO: 161 - protein, 6 aa, synthetic construct
GGGGLL

SEQ ID NO: 162 - protein, 6 aa, synthetic construct
LGGGGG

SEQ ID NO: 163 - protein, 6 aa, synthetic construct
GLGGGG

SEQ ID NO: 164 - protein, 6 aa, synthetic construct
GGLGGG

SEQ ID NO: 165 - protein, 6 aa, synthetic construct
GGGLGG

SEQ ID NO: 166 - protein, 6 aa, synthetic construct
GGGGLG

SEQ ID NO: 167 - protein, 6 aa, synthetic construct
GGGGGL

SEQ ID NO: 168 - protein, 6 aa, synthetic construct
GGGGGG

SEQ ID NO: 169 - protein, 4 aa, synthetic construct
  SITE 1: note = palmitic acid polyethylene glycol-8 glycine
GGSY

SEQ ID NO: 170 - protein, 5 aa, synthetic construct
  SITE 1: note = Acetyl palmitoyl lysine
KGGSY